(19) United States
(12) Patent Application Publication
(10) Pub. No.: US 2010/0144591 A1
(43) Pub. Date: Jun. 10, 2010

(54) BENZIMIDAZOLE DERIVATIVES AND METHODS OF USE THEREOF

(75) Inventors: Robert G. Aslanian, Rockaway, NJ (US); Jean E. Lachowicz, Berkeley Heights, NJ (US); Michael Y. Berlin, Flemington, NJ (US); Joyce J. Hwa, Westfield, NJ (US)

Correspondence Address: MERCK;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 12/527,500

(22) PCT Filed: Feb. 27, 2008
(86) PCT No.: PCT/US2008/002594

§ 371 Date: Jan. 13, 2010

Related U.S. Application Data
(60) Provisional application No. 60/904,453, filed on Mar. 2, 2007.

(51) Int. Cl. A61K 38/28; A61K 31/4545; A61K 31/506; A61K 31/497; A61K 31/5377; A61K 31/496; A61K 31/541; A61K 31/437; A61K 31/439; A61K 31/501; A61K 38/22; A61K 31/5415; A61K 31/60; A61P 3/10; A61P 3/04

(52) U.S. Cl. 514/3; 514/316; 514/275; 514/255.05; 514/234.5; 514/253.09; 514/228.2; 514/303; 514/304; 514/252.04; 514/12; 514/226.5; 514/165; 514/282

(57) ABSTRACT

The present invention relates to compounds of formula (I); compositions comprising the compounds, and methods of using the compounds to treat or prevent pain, diabetes, a diabetic complication, impaired glucose tolerance (IGT) or impaired fasting glucose (IGT) in a patient.

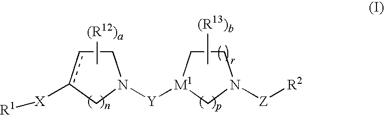

### FIELD OF THE INVENTION

The present invention relates to benzimidazole derivatives, compositions comprising the piperidine derivatives, and methods of using the benzimidazole derivatives to treat or prevent pain, diabetes, a diabetic complication, impaired glucose tolerance (IGT) or impaired fasting glucose (IFG) in a patient.

### BACKGROUND OF THE INVENTION

Diabetes refers to a disease process derived from multiple causative factors and is characterized by elevated levels of plasma glucose, or hyperglycemia in the fasting state or after administration of glucose during an oral glucose tolerance test. Persistent or uncontrolled hyperglycemia is associated with increased and premature morbidity and mortality. Abnormal glucose homeostasis is associated with alterations of lipid, lipoprotein and apolipoprotein metabolism and other metabolic and hemodynamic disease. As such, the diabetic patient is at increased risk of macrovascular and microvascular complications, including coronary heart disease, stroke, peripheral vascular disease, hypertension, nephropathy, neuropathy, and retinopathy. Accordingly, therapeutic control of glucose homeostasis, lipid metabolism and hypertension are critically important in the clinical management and treatment of diabetes mellitus.

There are two generally recognized forms of diabetes. In type 1 diabetes, or insulin-dependent diabetes mellitus (IDDM), patients produce little or no insulin, the hormone which regulates glucose utilization. In type 2 diabetes, or noninsulin dependent diabetes mellitus (NIDDM), patients often have plasma insulin levels that are the same or even elevated compared to nondiabetic subjects; however, these patients have developed a resistance to the insulin stimulating effect on glucose and lipid metabolism in the main insulin-sensitive tissue (muscle, liver and adipose tissue), and the plasma insulin levels, while elevated, are insufficient to overcome the pronounced insulin resistance.

Insulin resistance is not associated with a diminished number of insulin receptors but rather to a post-insulin receptor binding defect that is not well understood. This resistance to insulin responsiveness results in insufficient insulin activation of glucose uptake, oxidation and storage in muscle, and inadequate insulin repression of lipolysis in adipose tissue and of glucose production and secretion in the liver.

The available treatments for type 2 diabetes, which have not changed substantially in many years, have recognized limitations. While physical exercise and reductions in dietary intake of calories can dramatically improve the diabetic condition, compliance with this treatment is very poor because of well-entrenched sedentary lifestyles and excess food consumption, especially of foods containing high amounts of saturated fat. Increasing the plasma level of insulin by administration of sulfonylureas (e.g. tolbutamide and glipizide) or meglitinide, which stimulate the pancreatic [beta]-cells to secrete more insulin, and/or by injection of insulin when sulfonylureas or meglitinide become ineffective, can result in insulin concentrations high enough to stimulate the very insulin-resistant tissues. However, dangerously low levels of plasma glucose can result from administration of insulin or insulin secretagogues (sulfonylureas or meglitinide), and an increased level of insulin resistance due to the even higher plasma insulin levels can occur. The biguanides are a separate class of agents that can increase insulin sensitivity and bring about some degree of correction of hyperglycemia. These agents, however, can induce lactic acidosis, nausea and diarrhea.

The glitazones (i.e. 5-benzylthiazolidine-2,4-diones) are another class of compounds that have proven useful for the treatment of type 2 diabetes. These agents increase insulin sensitivity in muscle, liver and adipose tissue in several animal models of type 2 diabetes, resulting in partial or complete correction of the elevated plasma levels of glucose without occurrence of hypoglycemia. The glitazones that are currently marketed are agonists of the peroxisome proliferator activated receptor (PPAR), primarily the PPAR-gamma subtype. PPAR-gamma agonism is generally believed to be responsible for the improved insulin sensititization that is observed with the glitazones. Newer PPAR agonists that are being tested for treatment of Type II diabetes are agonists of the alpha, gamma or delta subtype, or a combination thereof, and in many cases are chemically different from the glitazones (i.e., they are not thiazolidinediones). Serious side effects (e.g. liver toxicity) have been noted in some patients treated with glitazone drugs, such as troglitazone.

Additional methods of treating the disease are currently under investigation. New biochemical approaches include treatment with alpha-glucosidase inhibitors (e.g. acarbose) and protein tyrosine phosphatase-1B (PTP-1B) inhibitors.

Compounds that are inhibitors of the dipeptidyl peptidase-IV (DPP-IV) enzyme are also under investigation as drugs that may be useful in the treatment of diabetes, and particularly type 2 diabetes.

Despite a widening body of knowledge concerning the treatment of diabetes, there remains a need in the art for small-molecule drugs with increased safety profiles and/or improved efficacy that are useful for the treatment of diabetes and related metabolic diseases. This invention addresses that need.

### SUMMARY OF THE INVENTION

The present invention provides novel compounds of formula I:

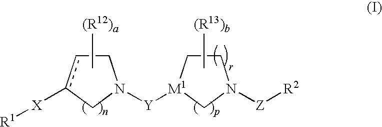

or a pharmaceutically acceptable salt, solvate, ester or prodrug thereof, wherein:

the dotted line represents an optional and additional bond;

M¹ is C(R³);

X is a bond or C₁-C₆ alkylene;

Y is —C(O)—, —C(S)—, —(CH₂)_{q}—, —C(O)NR⁴—, —C(O)CH₂—, —SO₂—, or —C(═N—CN)—NH—, such that when M¹ is N, Y is not —C(O)NR⁴— or —C(═N—CN)—NH—.

Z is a bond, C₁-C₆ alkylene, C₁-C₆ alkenylene, —C(O)—, —CH(CN)—, or —CH₂C(O)NR⁴—;

R¹ is

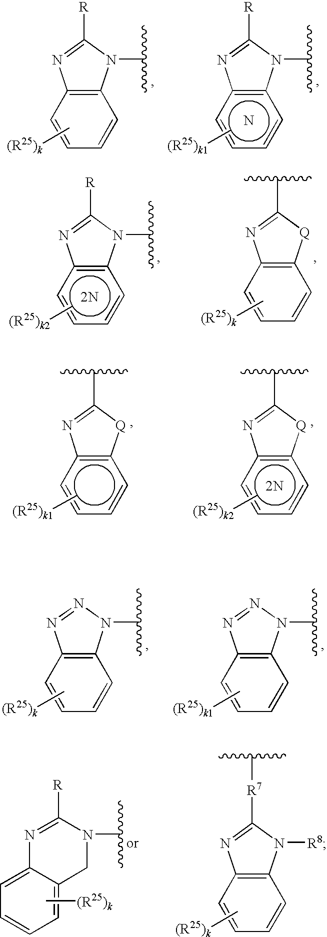

Q is —N(R⁸)—, —S— or —O—;

R is H, OH, C₁-C₆ alkyl, halo(C₁-C₆)alkyl-, C₁-C₆ alkoxy, (C₁-C₆)alkoxy-(C₁-C₆)alkyl-, (C₁-C₆)-alkoxy-(C₁-C₆)alkoxy, (C₁-C₆)alkoxy-(C₁-C₆)alkyl-SO₀₋₂, R³²-aryl(C₁-C₆)alkoxy-, R³²-aryl(C₁-C₆)alkyl-, R³²-aryl, R³²-aryloxy, R₃₂-heteroaryl, (C₃-C₆)cycloalkyl, (C₃-C₆)cyclo-alkyl-(C₁-C₆)alkyl, (C₃-C₆)cycloalkyl-(C₁-C₆)alkoxy, (C₃-C₆)cycloalkyl-oxy-, R³⁷-hetero-cycloalkyl, N(R³⁰)(R³¹)—(C₁-C₆)alkyl-, —N(R³⁰)(R³¹), —NH—(C₁-C₆)alkyl-O—(C₁-C₆)alkyl, —NHC(O)NH(R²⁹); R²⁹—S(O)₀₋₂—, halo(C₁-C₆)alkyl-S(O)₀₋₂—, N(R³⁰)(R³¹)—(C₁-C₆)alkyl-S(O)₀₋₂— or benzoyl;

R² is a six-membered heteroaryl ring having 1 or 2 heteroatoms independently selected from N or N—O, with the remaining ring atoms being carbon; a five-membered heteroaryl ring having 1, 2 or 3 heteroatoms independently selected from N, O or S, with the remaining ring atoms being carbon; R³²-quinolyl; R³²-aryl; heterocycloalkyl;

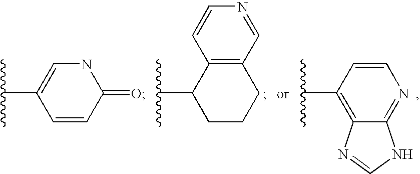

wherein said six-membered heteroaryl ring or said five-membered heteroaryl ring is optionally substituted by R⁶;

R³ is H, halo, C₁-C₆ alkyl, —OH or (C₁-C₆)alkoxy;

R⁴ is independently selected from the group consisting of hydrogen, C₁-C₆ alkyl, C₃-C₆ cycloalkyl, (C₃-C₆)cycloalkyl(C₁-C₆)alkyl, R³³-aryl, R³³-aryl(C₁-C₆)alkyl, and R³²-heteroaryl;

R⁵ is hydrogen, C₁-C₆ alkyl, —C(O)R²⁰, —C(O)₂R²⁰, —C(O)N(R²⁰)₂, (C₁-C₆)alkyl-SO₂—, or (C₁-C₆)alkyl-SO₂—NH—;

R⁶ is 1 to 3 substituents independently selected from the group consisting of —OH, halo, C₁-C₆ alkyl-, C₁-C₆ alkoxy, C₁-C₆ alkylthio, —CF₃, —NR⁴R⁵, phenyl, R³³-phenyl, NO₂, —CO₂R⁴, —CON(R⁴)₂,

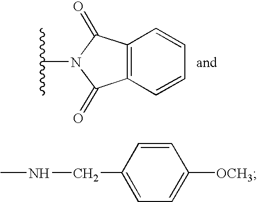

R⁷ is —N(R²⁹)—, —O— or —SO₀₋₂—;

R⁸ is H, C₁-C₆ alkyl, halo(C₁-C₆)alkyl-, (C₁-C₆)alkoxy-(C₁-C₆)alkyl-, R³²-aryl(C₁-C₆)alkyl-, R³²-aryl, R³²-heteroaryl, (C₃-C₆)cycloalkyl, (C₃-C₆)cycloalkyl-(C₁-C₆)alkyl, R³⁷-heterocycloalkyl, N(R³⁰)(R³¹)—(C₁-C₆)alkyl-, R²⁹—S(O)₂—, halo(C₁-C₆)alkyl-S(O)₂—, R²⁹—S(O)₀₋₁—(C₂-C₆)alkyl-, halo(C₁-C₆)alkyl-S(O)₀₋₁-(C₂-C₆)alkyl-;

R¹² is independently selected from the group consisting of C₁-C₆ alkyl, hydroxyl, C₁-C₆ alkoxy, or fluoro, provided that when R¹² is hydroxy or fluoro, then R¹² is not bound to a carbon adjacent to a nitrogen; or R¹² forms a C₁ to C₂ alkyl bridge from one ring carbon to another ring carbon;

R¹³ is independently selected from the group consisting of C₁-C₆ alkyl, hydroxyl, C₁-C₆ alkoxy, or fluoro, provided that when R¹³ is hydroxy or fluoro then R¹³ is not bound to a carbon adjacent to a nitrogen; or forms a C₁ to C₂ alkyl bridge from one ring carbon to another ring carbon; or R¹³ is ═O;

R²⁰ is independently selected from the group consisting of hydrogen, C₁-C₆ alkyl, or aryl, wherein said aryl group is optionally substituted with from 1 to 3 groups independently selected from halo, —CF₃, —OCF₃, hydroxyl, or methoxy; or when two R²⁰ groups are present, said two R²⁰ groups taken together with the nitrogen to which they are bound form a five or six membered heterocyclic ring;

R²² is C₁-C₆ alkyl, R³⁴-aryl or heterocycloalkyl;

R²⁴ is H, C₁-C₆ alkyl, —SO₂R²² or R³⁴-aryl;

R²⁵ is independently selected from the group consisting of C₁-C₆ alkyl, halo, —CF₃, —OH, C₁-C₆ alkoxy, (C₁-C₆)alkyl-C(O)—, aryl-C(O)—, N(R⁴)(R⁵)—C(O)—, N(R⁴)(R⁵)—S(O)₁₋₂—, halo-(C₁-C₆)alkyl- or halo-(C₁-C₆)alkoxy-(C₁-C₆)alkyl-;

R²⁹ is H, C₁-C₆ alkyl, C₃-C₆ cycloalkyl, R³⁵-aryl or R³⁵-aryl(C₁-C₆)alkyl-;

R³⁰ is H, C₁-C₆ alkyl-, R³⁵-aryl or R³⁵-aryl(C₁-C₆)alkyl-;

R³¹ is H, C₁-C₆ alkyl-, R³⁵-aryl, R³⁵-aryl(C₁-C₆)alkyl-, R³⁵-heteroaryl, (C₁-C₆)alkyl-C(O)—, R³⁵-aryl-C(O)—, N(R⁴)(R⁵)—C(O)—, (C₁-C₆)alkyl-S(O)₂— or R³⁵-aryl-S(O)₂—;

or R³⁰ and R³¹ together are —(CH₂)₄₋₅—, —(CH₂)₂—O—(CH₂)₂— or —(CH₂)₂—N(R³⁸)—(CH₂)₂— and form a ring with the nitrogen to which they are attached;

R³² is 1 to 3 substituents independently selected from the group consisting of H, —OH, halo, C₁-C₆ alkyl, C₁-C₆ alkoxy, R³⁵-aryl-O—, —SR²², —CF₃, —OCF₃, —OCHF₂, —NR⁴R⁵, phenyl, R³³-phenyl, NO₂, —CO₂R⁴, —CON(R⁴)₂, —S(O)₂R²², —S(O)₂N(R²⁰)₂, —N(R²⁴)S(O)₂R²², —CN, hydroxy-(C₁-C₆)alkyl-, —OCH₂CH₂OR²², and R³⁵-aryl(C₁-C₆)alkyl-O—, or two R³² groups on adjacent carbon atoms together form a —OCH₂O— or —O(CH₂)₂O— group;

R³³ is 1 to 3 substituents independently selected from the group consisting of C₁-C₆ alkyl, halo, —CN, —NO₂, —CF₃, —OCF₃, —OCHF₂ and —O—(C₁-C₆)alkyl;

R³⁴ is 1 to 3 substituents independently selected from the group consisting of H, halo, —CF₃, —OCF₃, —OH and —OCH₃.

R³⁵ is 1 to 3 substituents independently selected from hydrogen, halo, C₁-C₆ alkyl, hydroxy, C₁-C₆ alkoxy, phenoxy, —CF₃, —N(R³⁶)₂, —COOR²⁰ and —NO₂;

R³⁶ is independently selected form the group consisting of H and C₁-C₆ alkyl;

R³⁷ is 1 to 3 substituents independently selected from hydrogen, halo, C₁-C₆ alkyl, hydroxy, C₁-C₆ alkoxy, phenoxy, —CF₃, —N(R³⁶)₂, —COOR²⁰, —C(O)N(R²⁹)₂ and —NO₂, or R³⁷ is one or two ═O groups;

R³⁸ is H, C₁-C₆ alkyl, R³⁵-aryl, R³⁵-aryl(C₁-C₆)alkyl-, (C₁-C₆)alkyl-SO₂ or halo(C₁-C₆)alkyl-SO₂—;

a is 0, 1 or 2;

b is 0, 1 or 2;

k is 0, 1, 2, 3 or 4;

k1 is 0, 1, 2 or 3;

k2 is 0, 1 or 2;

n is 1 or 2;

p is 1, 2 or 3;

q is an integer ranging from 1 to 5; and

r is an integer ranging from 0 to 3,

such that: (i) when M¹ is N, p is not 1; (ii) when r is 0, M¹ is C(R³); and (iii) the sum of p and r is 3.

In another another aspect, the invention provides a method for treating pain, diabetes, a diabetic complication, impaired glucose tolerance or impaired fasting glucose (each being a “Condition”) in a patient, comprising administering to the patient an effective amount of one or more Compounds of Formula (I).

In a further aspect, the invention provides compositions comprising one or more Compounds of Formula (I), an additional therapeutic agent that is not a Compound of Formula (I), and a pharmaceutically acceptable carrier, wherein the amounts of the one or more Compounds of Formula (I) and the additional therapeutic agent are together effective to treat a Condition in a patient.

### BRIEF DESCRIPTION OF THE FIGURES

FIG. 1 shows the effect of Compound 174 and rosiglitazone on non-fasting glucose levels in STZ-induced type 2 diabetic mice. The leftmost black solid bar represents diabetic control mice, the second from left black solid bar represents mice treated for one week with rosiglitazone at 5 mg/kg/day; the third from left black solid bar represents mice treated for one week with Compound 174 at 10 mg/kg/day; the fourth from left black solid bar represents mice treated for one week with Compound 174 at 1 mg/kg/day; and the white bar represents nondiabetic control mice. The y-axis indicates non-fasting glucose levels (mg/dl).

FIG. 2 shows the effect of Compound 174 on plasma HbA1c levels in a rat model of diabetes. The leftmost bar represents untreated control rats, the middle gray bar represents rats treated with Compound 174 (3 mg/kg/day in diet, two weeks of treatment), and the rightmost black bar represents rats treated with Compound 174 (10 mg/kg/day in diet, two weeks of treatment). The y-axis represents the percent change in HbA1c levels of the test animals (mg/dl) due to treatment.

### DETAILED DESCRIPTION OF THE INVENTION

As used above, and throughout this disclosure, the following terms, unless otherwise indicated, shall be understood to have the following meanings:

A “patient” is a human or non-human mammal. In one embodiment, a patient is a human. In another embodiment, a patient is a non-human mammal, including, but not limited to, a monkey, dog, baboon, rhesus, mouse, rat, horse, cat or rabbit. In another embodiment, a patient is a companion animal, including but not limited to a dog, cat, rabbit, horse or ferret. In one embodiment, a patient is a dog. In another embodiment, a patient is a cat.

The term “obesity” as used herein, refers to a patient being overweight and having a body mass index (BMI) of 25 or greater. In one embodiment, an obese patient has a BMI of 25 or greater. In another embodiment, an obese patient has a BMI from 25 to 30. In another embodiment, an obese patient has a BMI greater than 30. In still another embodiment, an obese patient has a BMI greater than 40.

The term “impaired glucose tolerance” as used herein, is defined as a two-hour glucose level of 140 to 199 mg per dL (7.8 to 11.0 mmol) as measured using the 75-g oral glucose tolerance test. A patient is said to be under the condition of impaired glucose tolerance when he/she has an intermediately raised glucose level after 2 hours, wherein the level is less than would qualify for type 2 diabetes mellitus.

The term “impaired fasting glucose” as used herein, is defined as a fasting plasma glucose level of 100 to 125 mg/dL; normal fasting glucose values are below 100 mg per dL.

The term “effective amount” as used herein, refers to an amount of Compound of Formula (I) and/or an additional therapeutic agent, or a composition thereof that is effective in producing the desired therapeutic, ameliorative, inhibitory or preventative effect when administered to a patient suffering from a Condition. In the combination therapies of the present invention, an effective amount can refer to each individual agent or to the combination as a whole, wherein the amounts of all agents administered are together effective, but wherein the component agent of the combination may not be present individually in an effective amount.

The term “alkyl,” as used herein, refers to an aliphatic hydrocarbon group which may be straight or branched and which contains from about 1 to about 20 carbon atoms. In one embodiment, an alkyl group contains from about 1 to about 12 carbon atoms. In another embodiment, an alkyl group contains from about 1 to about 6 carbon atoms. Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or optionally substituted by one or more substituents which may be the same or different, each substituent being independently selected from the group consisting of halo, alkyl, aryl, cycloalkyl, cyano, —OH, —O-alkyl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkyl group is unsubstituted. In another embodiment, an alkyl group is linear. In another embodiment, an alkyl group is branched.

The term “alkylene,” as used herein, refers to an alkyl group, as defined above, wherein one of the alkyl group's hydrogen atoms has been replaced with a bond. Non-limiting examples of alkylene groups include —CH₂—, —CH₂CH₂—, —CH₂CH₂CH₂—, —CH₂CH₂CH₂CH₂—, —CH(CH₃)CH₂CH₂— and —CH₂CH(CH₃)CH₂—. In one embodiment, an alkylene group has from 1 to about 6 carbon atoms. In another embodiment, an alkylene group is branched. In another embodiment, an alkylene group is linear.

The term “aryl,” as used herein, refers to an aromatic monocyclic or multicyclic ring system comprising from about 6 to about 14 carbon atoms. In one embodiment, an aryl group contains from about 6 to about 10 carbon atoms. An aryl group can be optionally substituted with one or more “ring system substituents” which may be the same or different, and are as defined herein below. Non-limiting examples of illustrative aryl groups include phenyl and naphthyl. In one embodiment, an aryl group is unsubstituted. In another embodiment, an aryl group is phenyl.

The term “alkylaryl” as used herein, refers to an aryl group, as defined above, joined to an alkyl group, as defined above, wherein an alkylaryl group is bound to the rest of the molecule via it's aryl moiety.

The term “arylalkyl” as used herein, refers to an aryl group, as defined above, joined to an alkyl group, as defined above, wherein an arylalkyl group is bound to the rest of the molecule via it's alkyl moiety. In one embodiment, an arylalkyl group is a benzyl group.

The term “cycloalkyl,” as used herein, refers to a non-aromatic mono- or multicyclic carbocyclic ring system comprising from about 3 to about 10 ring carbon atoms. In one embodiment, a cycloalkyl contains from about 5 to about 10 ring carbon atoms. In another embodiment, a cycloalkyl contains from about 5 to about 7 ring atoms. Non-limiting examples of illustrative monocyclic cycloalkyls include cyclopropyl, cyclobutyl, cyclopentyl, cyclohexyl, cycloheptyl and cyclooctyl. Non-limiting examples of illustrative multicyclic cycloalkyls include 1-decalinyl, norbornyl and adamantyl. A cycloalkyl group can be optionally substituted with one or more “ring system substituents” which may be the same or different, and are as defined herein below. In one embodiment, a cycloalkyl group is unsubstituted.

The term “halo” as used herein, refers to —F, —Br or —I.

The term “haloalkyl” as used herein, refers to an alkyl group, as defined above, wherein one or more of the alkyl group's hydrogen atoms have been independently replaced with —F, —Cl, —Br or —I. Non-limiting illustrative examples of haloalkyl groups include —CH₂F, —CHF₂, —CF₃, —CH₂CHF₂, —CH₂CHF₃, —CCl₃, —CHCl₂, —CH₂Cl, and —CH₂CHCl₃.

The term “heteroaryl,” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. A heteroaryl group can be optionally substituted by one or more “ring system substituents” which may be the same or different, and are as defined herein below. A heteroaryl group can be joined via a ring carbon atom or a ring nitrogen atom and any ring nitrogen atom of a heteroaryl group can be optionally oxidized to the corresponding N-oxide. The term “heteroaryl” also encompasses a heteroaryl group, as defined above, which has been fused to a benzene ring. Non-limiting examples of illustrative heteroaryl groups include pyridyl (e.g., 2-, 3-, or 4-pyridyl), pyridyl N-oxide (e.g., 2-, 3-, or 4-pyridyl N-oxide), pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like. In one embodiment, a heteroaryl has from 5 to 7 ring atoms. In another embodiment, a heteroaryl has 5 or 6 ring atoms. In another embodiment, a heteroaryl has 5 ring atoms. In still another embodiment, a heteroaryl has 6 ring atoms.

The term “heterocycloalkyl,” as used herein, refers to a non-aromatic, saturated monocyclic or multicyclic ring system comprising from 3 to about 10 ring atoms, wherein from 1 to 4 of the ring atoms are independently O, S or N and the remainder of the ring atoms are carbon atoms. In one embodiment, a heterocycloalkyl group has from about 5 to about 10 ring atoms. In another embodiment, a heterocycloalkyl group has 5 or 6 ring atoms. There are no adjacent oxygen and/or sulfur atoms present in the ring system. Any —NH group in a heterocycloalkyl ring may exist protected such as, for example, as an —N(Boc), —N(CBz), —N(Tos) group and the like; such protected heterocycloalkyl groups are considered part of this invention. A heterocycloalkyl group can be optionally substituted by one or more “ring system substituents” which may be the same or different, and are as defined herein below. The nitrogen or sulfur atom of the heterocyclyl can be optionally oxidized to the corresponding N-oxide, S-oxide or S,S-dioxide. Non-limiting examples of illustrative monocyclic heterocycloalkyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. A ring carbon atom of a heterocycloalkyl group may be functionalized as a carbonyl group. An illustrative example of such a heterocycloalkyl group is is pyrrolidonyl:

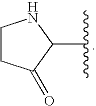

The symbol

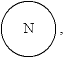

when present inside a ring, indicates that one of the ring's non-fused carbon atoms is replaced with a nitrogen atom. For example, in the structure:

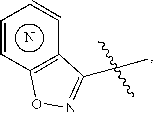

the presence of the symbol

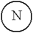

inside the 6-membered ring indicates that a nitrogen atom that is located at one of the 4 non-fused positions of the 6-membered ring, i.e., positions 1, 2, 3 or 4 indicated below:

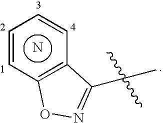

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound' or “stable structure” is meant a compound that is sufficiently robust to survive isolation to a useful degree of purity from a reaction mixture, and formulation into an efficacious therapeutic agent.

The term “ring system substituent,” as used herein, refers to a substituent group attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, arylalkyl, alkylaryl, heteroarylalkyl, heteroarylalkenyl, heteroarylalkynyl, alkylheteroaryl, —OH, hydroxyalkyl, —O-alkyl, -alkylene-O-alkyl, —O-aryl, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, arylalkylthio, heteroarylalkylthio, cycloalkyl, heterocyclyl, —O—C(O)-alkyl, —O—C(O)-aryl, —O—C(O)-cycloalkyl, —C(═N—CN)—NH₂, —C(═NH)—NH₂, —C(═NH)—NH(alkyl), Y₁Y₂N—, Y₁Y₂N-alkyl-, Y₁Y₂NC(O)— and Y₁Y₂NSO₂—, wherein Y₁ and Y₂ can be the same or different and are independently selected from the group consisting of hydrogen, alkyl, aryl, cycloalkyl, and arylalkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

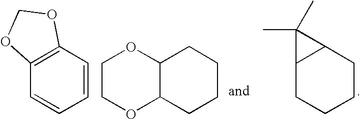

Any atom with unsatisfied valences in the text, schemes, examples and tables herein is assumed to have the sufficient number of hydrogen atom(s) to satisfy the valences.

The term “one or more Compounds of Formula (I)” as used herein in connection with the treatment or prevention of a Condition in a patient means that at least one Compound of Formula (I) is administered to the patient. In one embodiment, the phrase “one or more” refers to one Compound of Formula (I). In another embodiment, the phrase “one or more” refers to two Compounds of Formula (I).

The term “coxib” as used herein, refers to an agent that is an inhibitor of the COX-2 enzyme. A coxib may inhibit both the COX-1 and COX-2 enzymes, or may selectively inhibit the COX-2 enzyme.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, N.Y.

When any variable (e.g., aryl, heterocycle, R², etc.) occurs more than one time in any constituent or in Formula (I), its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to provide a Compound of Formula (I) or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a Compound of Formula (I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl, and the like.

Similarly, if a Compound of Formula (I) contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a —OH group of the hemiacetal form of a carbohydrate), and the like.

If a Compound of Formula (I) incorporates an amine functional group, a prodrug can be formed by the replacement of a hydrogen atom in the amine group with a group such as, for example, R-carbonyl, RO-carbonyl, NRR′-carbonyl where R and R′ are each independently (C1-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of illustrative solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS Pharm Sci Tech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

The Compounds of Formula (I) can form salts which are also within the scope of this invention. Reference to a Compound of Formula (I) herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a Compound of Formula (I) contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula (I) may be formed, for example, by reacting a Compound of Formula (I) with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), arylalkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the —OH groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), arylalkyl (for example, benzyl), aryloxyalkyl (for example, phenoxymethyl), aryl (for example, phenyl optionally substituted with, for example, halo, C₁₋₄alkyl, or C₁₋₄alkoxy or amino); (2) sulfonate esters, such as alkyl- or arylalkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

Compound of Formula (I), and salts, solvates, hydrates, esters and prodrugs thereof, may exist in their tautomeric form (for example, as an amide or imino ether, or in keto-enol form). All such tautomeric forms are considered equivalent and are contemplated herein as part of the present invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the Compounds of Formula (I) may be atropisomers (e.g., substituted biaryls) and are considered as part of this invention. Enantiomers can also be separated by use of chiral HPLC column.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a Compound of Formula (I) incorporates a double bond or a fused ring, both the cis- and trans-forms, as well as mixtures, are embraced within the scope of the invention. Also, for example, all keto-enol and imine-enamine forms of the compounds are included in the invention.).

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled Compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled Compounds of Formula (I) can generally be prepared using synthetic chemical procedures analogous to those disclosed herein for making the Compounds of Formula (I), by substituting an appropriate isotopically labelled starting material or reagent for a non-isotopically labelleds starting material or reagent.

Polymorphic forms of the Compound of Formula (I), and of the salts, solvates, hydrates, esters and prodrugs of the Compound of Formula (I), are intended to be included in the present invention.

The compounds of this invention can be ligands for the histamine H₃ receptor. In one embodiment, the Compounds of Formula (I) are antagonists of the H₃ receptor.

The following abbreviations are used herein and have the following meanings: Me=methyl; Et=ethyl; Bu=butyl; Pr=propyl; Ph=phenyl; t-BOC=tert-butylcarbonyl; CBZ=carbobenzyloxy; Ac=acetyl; DCC=dicyclohexylcarbodiimide; DMAP=4-dimethylaminopyridine; DMF=dimethylformamide; EDCI=1-(3-dimethylaminopropyl)-3-ethylcarbodiimide; HATU=O-(7-Azabenzotriazol-1-yl)-N,N,N′,N′-tetramethyl uronium hexafluorophosphate; HOBT=1-hydroxybenzotriazole; LAH=lithium aluminum hydride; LDA=lithium diisopropylamide; NaBH(OAc)₃=sodium triacetoxyborohydride; NBS=N-bromosuccinimide; PPA=polyphosphoric acid; RT=room temperature; TBAF=tetrabutylammonium fluoride; TBDMS=t-butyldimethylsilyl; TMEDA=N,N,N′,N′-tetramethylethylenediamine; TEMPO=2,2,6,6-tetramethyl-1-piperidinyloxy, free radical; TLC=thin layer chromatography; HRMS=High Resolution Mass Spectrometry; LRMS=Low Resolution Mass Spectrometry; nM=nanomolar; Ki=Dissociation Constant for bromosuccinimide; PPA=polyphosphoric acid; RT=room temperature; TBAF=tetrabutylammonium fluoride; TBDMS=t-butyldimethylsilyl; TMEDA=N,N,N′,N′-tetramethylethylenediamine; TEMPO=2,2,6,6-tetramethyl-1-piperidinyloxy, free radical; TLC=thin layer chromatography; HRMS=High Resolution Mass Spectrometry; LRMS=Low Resolution Mass Spectrometry; nM=nanomolar; Ki=Dissociation Constant for substrate/receptor complex; pA2=−log EC₅₀, as defined by J. Hey, Eur. J. Pharmacol., (1995), Vol. 294, 329-335; and Ci/mmol=Curie/mmol (a measure of specific activity).

### The Compounds of Formula (I)

The present invention provides uses of, and compositions comprising, compounds having the formula:

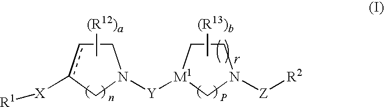

and pharmaceutically acceptable salts, solvates, esters and prodrugs thereof, wherein R¹, R², R¹², R¹³, X, Y, Z, M¹, a, b, n and p are defined above for the Compounds of Formula (I).

In one embodiment, R¹ is

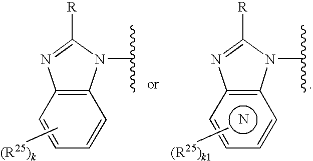

In another embodiment, R¹ is

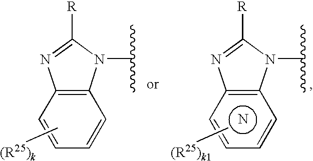

wherein R is alkoxy, alkoxyalkoxy, alkylthio, heteroaryl or R³²-aryl. In one embodiment, R is a mono- or di-halo substituted phenyl group.

In another embodiment, R¹ is

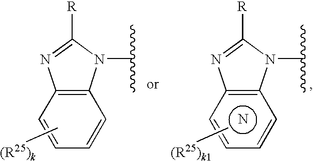

wherein R is —OCH₃, —OCH₂CH₃, —OCH((CH₃)₂, —SCH₃, —SCH₂CH₃, pyridyl (especially 2-pyridyl), pyrimidyl, pyrazinyl, furanyl, oxazolyl or R³²-phenyl.

In another embodiment, R²⁵, when present, is halo or —CF₃ and k is 0 or 1.

In still another embodiment, R¹ is:

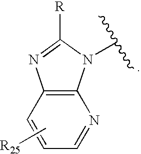

In a further embodiment, R¹ is:

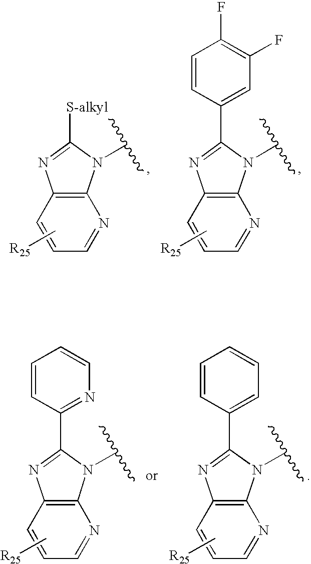

In another embodiment, R¹ is:

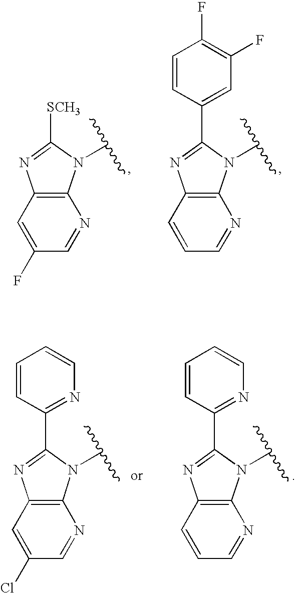

In one embodiment; R² is a six-membered heteroaryl.

In another embodiment, R² is a six-membered heteroaryl having one substituent.

In another embodiment, R² is a six-membered heteroaryl substituted with —NH₂.

In still another embodiment, R² is pyrimidyl or pyridyl.

In yet another embodiment, R² is pyrimidyl or pyridyl, each of which is substituted with —NH₂.

In a further embodiment, R² is

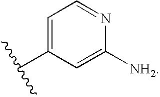

In one embodiment, X is a bond.

In another embodiment, X is C₁-C₆ alkylene.

In one embodiment, Y is —C(O)—.

In another embodiment, Y is —C(S)—.

In another embodiment, Y is —(CH₂)_{q}—.

In still another embodiment, Y is —CH₂—.

In one embodiment, Z is C₁-C₆ alkylene.

In another embodiment, Z is C₁-C₆ alkenylene.

In another embodiment, Z is —C(O)—.

In still another embodiment, Z is —CH₂—.

In one embodiment, M¹ is CH.

In another embodiment, M¹ is CF.

In another embodiment, M¹ is N.

In one embodiment, n is 2.

In another embodiment, p is 2.

In another embodiment, r is 1.

In one embodiment, a is 0

In another embodiment, b is 0.

In another embodiment, a and b are each 0.

In one embodiment, M¹ is CH, n is 2, p is 2 and r is 1.

In another embodiment, M¹ is CH and Y is —C(O)—.

In one embodiment, M¹ is CH, Y is —C(O)—, n is 2, p is 2 and r is 1.

In another embodiment, M¹ is CH, Y is —C(O)—, n is 2, p is 2, r is 1 and a and b are each 0.

In one embodiment, X is a bond; R¹ is optionally substituted benzimidazolyl or 4-azabenzimidazolyl; and R² is a six-membered heteroaryl.

In another embodiment, X is a bond; R¹ is optionally substituted 4-azabenzimidazolyl; Z is —CH₂— and R² is pyridyl or pyrimidyl.

In another embodiment, X is a bond, Z is —CH₂—, R¹ is

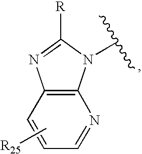

and R² is pyridyl or pyrimidyl.

In still another embodiment, X is a bond, Z is —CH₂—, R¹ is

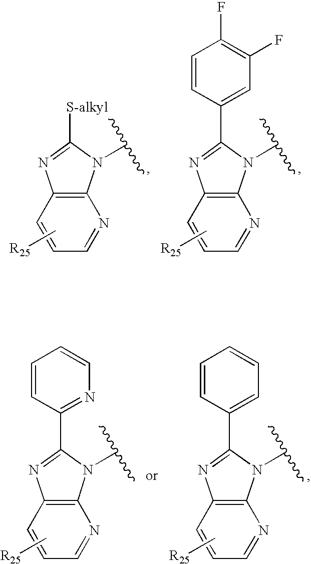

and R² is pyridyl or pyrimidyl.

In yet another embodiment, X is a bond, Z is —CH₂—, R¹ is

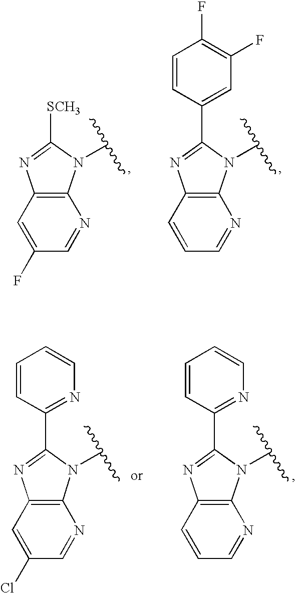

### and R² is

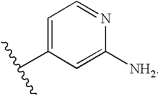

In one embodiment, the compounds of formula (I) have the formula (Ia):

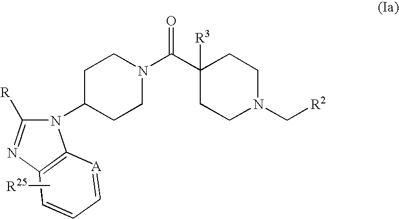

wherein R, R², R³, R²⁵ are defined above for the compounds of formula (I) and A is N or CH.

In one embodiment, for the compounds of formula (Ia), R is R³²-aryl. In another embodiment, R is R³²-phenyl. In another embodiment, R is phenyl, substituted with one or more halo groups. In still another embodiment, R is phenyl, substituted with one or more fluoro groups. In a further embodiment, R is 3,4-difluorophenyl.

In one embodiment, for the compounds of formula (Ia), A is N. In another embodiment, A is CH.

In one embodiment, for the compounds of formula (Ia), R³ is H. In another embodiment, R³ is —OH or halo. In another embodiment, R³ is —F.

In one embodiment, for the compounds of formula (Ia), R² is a six-membered heteroaryl. In another embodiment, for the compounds of formula (Ia), R² is pyridyl or pyrimindinyl. In another embodiment, for the compounds of formula (Ia), R² is:

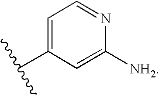

In one embodiment, for the compounds of formula (Ia), R is R³²-aryl and A is N.

In another embodiment, for the compounds of formula (Ia), R is R³²-aryl, A is N, and R³ is H or F.

In still another embodiment, for the compounds of formula (Ia), R is R³²-aryl, A is N, R³ is H or F and R² is a six-membered heteroaryl.

In another embodiment, for the compounds of formula (Ia), R is R³²-phenyl, A is N, R³ is H or F and R² is a six-membered heteroaryl.

In another embodiment, for the compounds of formula (Ia), R is R³²-phenyl, A is N, R³ is H or F, and R² is pyridyl or pyrimidinyl.

In yet another embodiment, for the compounds of formula (Ia), R is phenyl, substituted with one or more halo groups; A is N; R³ is H or F; and R² is a six-membered heteroaryl.

In a further embodiment, for the compounds of formula (Ia), R is phenyl, substituted with one or more halo groups; A is N; R³ is H or F; and R² is pyridyl.

In a further embodiment, for the compounds of formula (Ia), R is phenyl, substituted with one or more halo groups; A is N; R³ is H or F; and R² is:

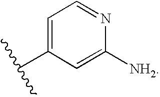

Illustrative examples of the compounds of formula (I) include, but are not limited to, compounds 1-666 as set forth in the Examples and compound tables below, and pharmaceutically acceptable salts, solvates, esters and prodrugs thereof.

In one embodiment, the compound of formula (I) is

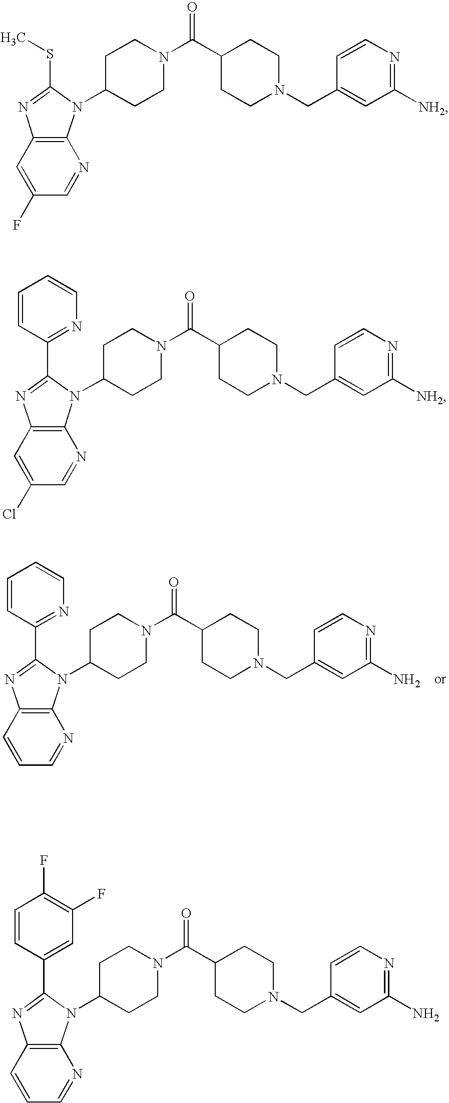

and pharmaceutically acceptable salts, solvates, esters and prodrugs thereof.

In one embodiment, for the compounds of formula (I), variables R¹, R², R¹², R¹³, X, Y, Z, M¹, a, b, n and p are selected independently from each other.

In another embodiment, the compounds of formula (I) are in purified form.

In one embodiment, for the compounds of formula (Ia), variables R, R², R³, R²⁵ and A are selected independently from each other.

In another embodiment, the compounds of formula (Ia) are in purified form.

### Methods for Making the Compounds of Formula (I)

Methods useful for making the Compounds of Formula (I) are set forth in the Examples below and generalized in Schemes 1-7.

Scheme 1 shows a method useful for making the compounds of formula IA, wherein R¹ is 1-benzimidazolyl or 2-benzamidazolyl and R⁷ is a bond or alkyl.

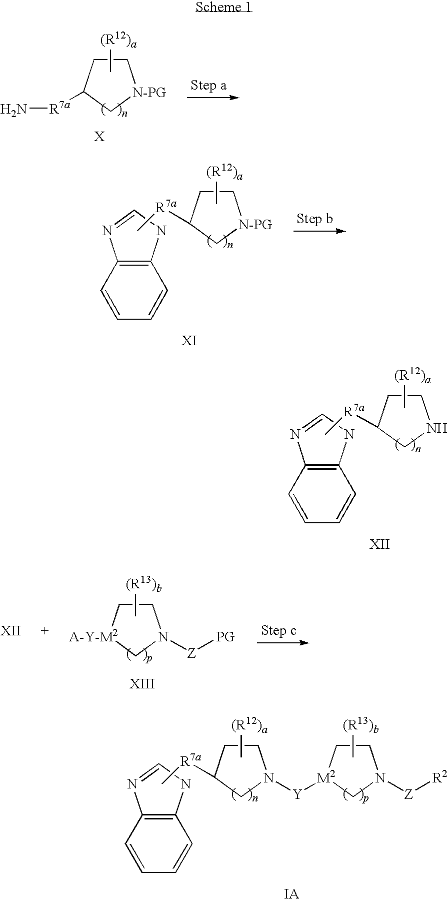

wherein R^{7a} is a bond or alkyl, PG is a secondary amine protecting group, and the remaining variables are as defined above for the compounds of formula (I).
Step a: The free amino group of a suitably monoprotected diamine of formula X can be alkylated or arylated with an alkyl or aryl halide. The resulting intermediate compound can then be cyclized with an appropriate carbonyl equivalent to form a compound of formula XI. Suitable amino protecting groups include methyl, benzyl, butoxycarbonyl, or ethoxycarbonyl. A suitable halide for alkylation is a substituted aromatic compound or a substituted hetero-aromatic compound as described by Henning et al, J. Med. Chem. 30, (1987), 814-819.
Step b: The protected amine of formula XI can be deprotected using methods known to those skilled in the art. A suitable method for methyl deprotection includes, but is not limited to, reaction with a haloformate or the like. A suitable method for benzyl deprotection includes, but is not limited to, cleavage with hydrogen at or above atmospheric pressure and a catalyst such as palladium. A suitable method for carbamate deprotection includes, but is not limited to, treatment with an acid.
Step c: An amine of formula XII can be reacted with an activated functional group Y of formula XIII to form the bond between the nitrogen and functional group Y in formula IA. When Y is a carbonyl group and M² is carbon, activation can be via a halide (i.e. acid chloride intermediate) or other coupling reagents (EDCI, DCC, HATU, or like). Suitable reaction conditions may require a base such as triethylamine or N,N-diisopropylethylamine.

Those skilled in the art of organic synthesis will appreciate that the method of Scheme 1 can be modified to prepare compounds wherein the benzene ring of the benzimidazolyl group can be substituted, as well as the aza-benzimidazoles compounds (i.e., compounds wherein R¹ is other than benzimidazolyl as defined above) and the benzoxazolyl and benzothiazolyl derivatives.

Scheme 2 illustrates an alternative method useful for making the compounds of formula IA wherein R¹ is 1-benzimidazolyl or 2-benzimidazolyl and X is a bond or alkyl. Similar procedures can be used to prepare compounds wherein the benzene ring of the benzimidazolyl group can be substituted, as well as the aza-benzimidazoles compounds (i.e., compounds wherein R¹ is other than benzimidazolyl as defined above).

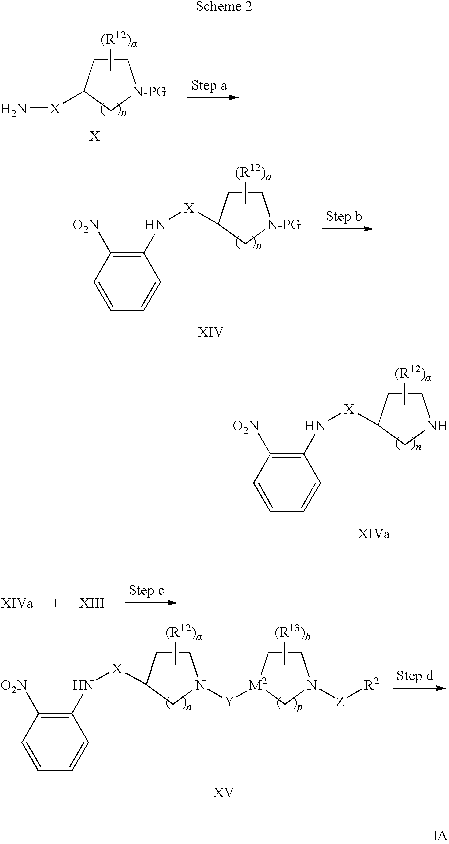

wherein R^{7a} is a bond or alkyl, PG is a secondary amino protecting group, and the remaining variables are as defined above for the compounds of formula (I).
Step a: A suitably monoprotected diamine of formula X can be alkylated or arylated with a halide to form a compound of formula XIV. Suitable protecting groups are methyl, benzyl, butoxycarbonyl, and ethoxycarbonyl. A suitable halide for alkylation is a substituted aromatic compound or a substituted hetero-aromatic compound as described by Henning et al.

### Step b:

(1) The protected amine of formula XIV can be deprotected using methods known to those skilled in the art. A suitable method for methyl deprotection includes, but is not limited to, reaction with a haloformate or the like. A suitable method for benzyl deprotection includes, but is not limited to, cleavage with hydrogen at or above atmospheric pressure and a catalyst such as palladium. A suitable method for carbamate deprotection includes, but is not limited to, treatment with an acid.
Step c: The resulting amine from Step b can be reacted with an activated functional group Y of formula XIII to form the bond between the nitrogen and functional group Y to obtain the compound of formula XV. When Y is a carbonyl group and M² is carbon, activation can be via a halide (i.e. acid chloride intermediate) or other coupling reagents (EDCI, DCC, HATU, or the like). Suitable reaction conditions may require a base such as triethylamine, N,N-diisopropylethylamine, pyridine, or the like.
Step d: After reduction of formula XV, the resulting compound can be reacted with a carbonyl equivalent to give the cyclized compound of formula IA. The reduction conditions can be hydrogen in the presence of catalyst, metal in the presence of an acid or a base, or other reduction reagent. The cyclization can be performed in acidic or basic conditions.

Scheme 3 shows a method useful for making the compounds of formula IB.

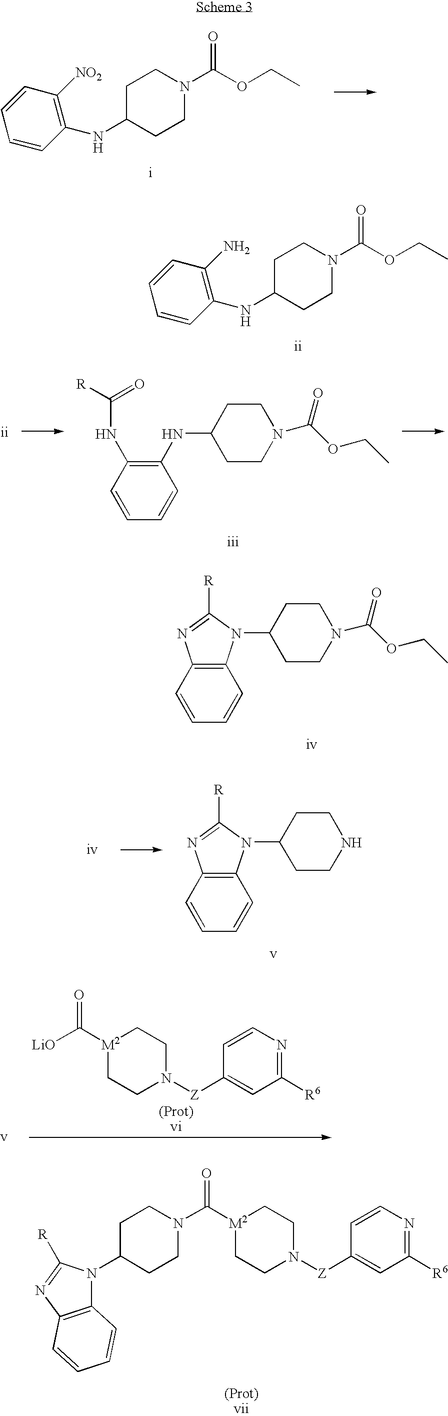

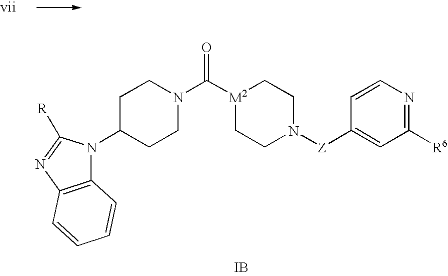

Scheme 4 shows an alternative method useful for making the compounds of formula IB.

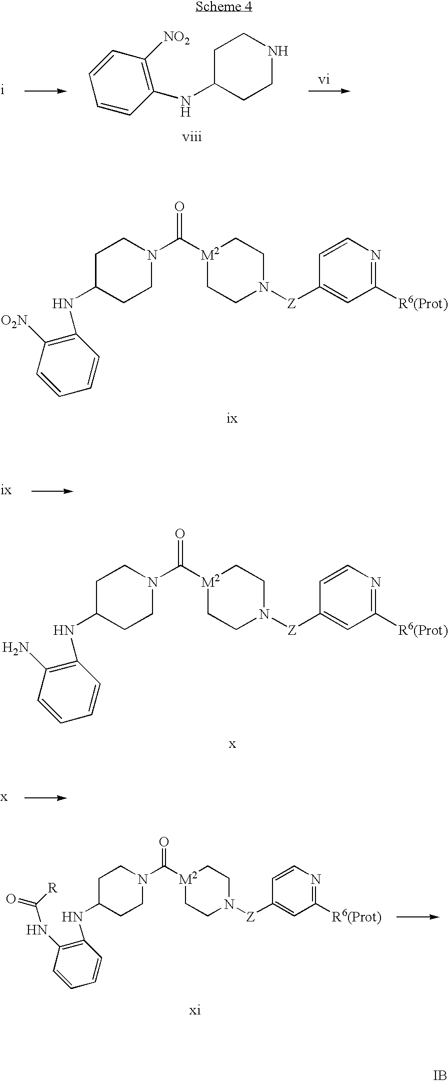

Scheme 5 shows another alternative method useful for making the compounds of formula IB.

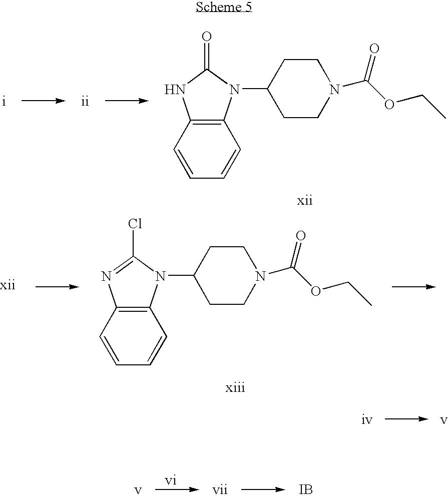

Scheme 6 shows a method useful for making the compounds of formula IC.

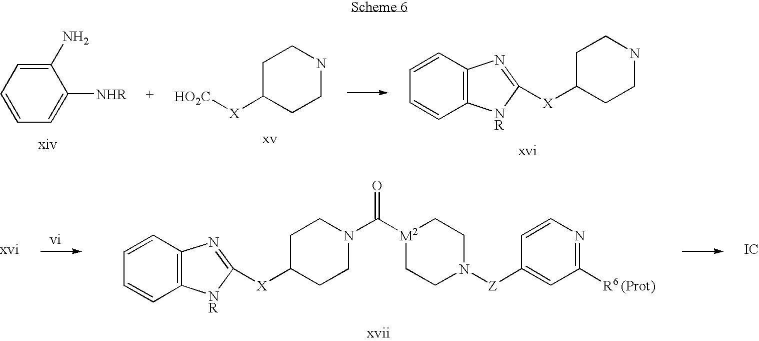

Scheme 7 shows a method useful for making the compounds of formula ID.

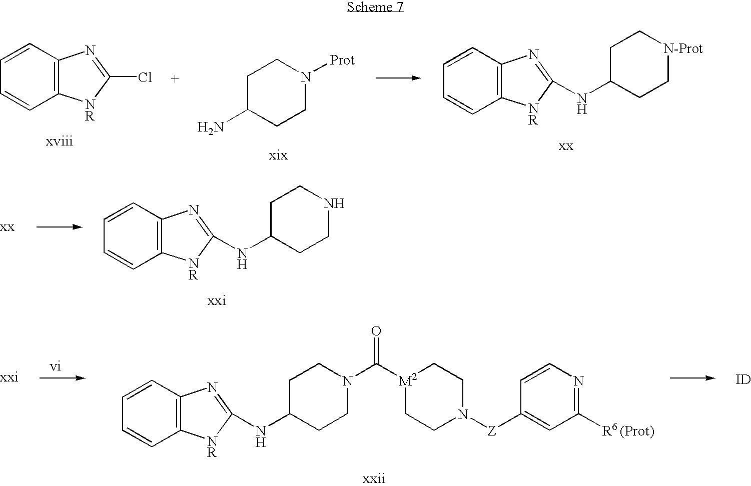

Those skilled in the art of organic synthesis will appreciate that similar procedures can be used to prepare compounds wherein the benzene ring of the benzimidazolyl group is substituted, R² is other than pyridyl, and aza-benzimidazoles compounds (i.e., compounds wherein R¹ is other than benzimidazolyl as defined above).

Specifically exemplified compounds were prepared as described in the examples below, from starting materials known in the art or prepared as described below. These examples are being provided to further illustrate the present invention. They are for illustrative purposes only; the scope of the invention is not to be considered limited in any way thereby.

The compounds of the present invention can be prepared by a number of methods that will be evident to one skilled in the art of organic synthesis. Useful methods for making the Compounds of Formula (I) include, but are not limited to, the general and specific synthetic procedures described herein. One skilled in the art of organic synthesis will recognize that the procedures set forth herein can be used to make the entire scope of the Compounds of Formula (I) by using appropriate starting materials and reagents. Additionally, one skilled in the art will recognize that the methods useful for making the compounds is not limited to that which is set forth herein and that in some cases the order of steps in a particular synthetic scheme must be selected such that functional group incompatibilities are avoided.

The starting material and reagents used in preparing compounds described are either available from commercial suppliers such as Aldrich Chemical Co. (Wisconsin, USA) and Acros Organics Co. (New Jersey, USA) or were prepared by literature methods known to those skilled in the art.

One skilled in the art will recognize that the synthesis of compounds of formula I may require the construction of carbon-nitrogen bond. Methods include but are not limited to the use of a substituted aromatic compound or heteroaromatic compound and amine at 0° C. to 200° C. The reaction may be carried out neat or in a solvent. Suitable solvents for the reaction are halogenated hydrocarbons, ethereal solvents, toluene, dimethylformamide and the like.

One skilled in the art will recognize that the synthesis of compounds of formula I may require the construction of heterocycle. Methods include but are not limited to the use of a diamino compound and a carbonyl equivalent at 0° C. to 200° C. The reaction may be carried out in acidic, basic or neutral conditions. Suitable solvents for the reaction are water, halogenated hydrocarbons, ethereal solvents, alcoholic solvents, toluene, ketones, dimethylformamide and the like.

One skilled in the art will recognize that the synthesis of compounds of formula I may require the need for the protection of certain functional groups (i.e. derivatization for the purpose of chemical compatibility with a particular reaction condition). A suitable protecting group for an amine is methyl, benzyl, ethoxyethyl, t-butoxycarbonyl, phthaloyl and the like which can appended to and removed by literature methods known to those skilled in the art.

One skilled in the art will recognize that the synthesis of compounds of formula I may require the construction of an amide bond. Methods include but are not limited to the use of a reactive carboxy derivative (e.g. acid halide) or the use of an acid with a coupling reagent (e.g. EDCI, DCC, HATU) with an amine at 0° C. to 100° C. Suitable solvents for the reaction are halogenated hydrocarbons, ethereal solvents, dimethylformamide and alike.

One skilled in the art will recognize that the synthesis of compounds of formula I may require the reduction of a functional group. Suitable reducing reagents for the reaction include NaBH₄, lithium aluminum hydride, diborane and the like at −20° C. to 100° C. Suitable solvents for the reaction are halogenated hydrocarbons, ethereal solvents, and the like.

The starting materials and the intermediates of the reaction may be isolated and purified if desired using conventional techniques, including but not limited to filtration, distillation, crystallization, chromatography and alike. Such materials can be characterized using conventional means, including physical constants and spectral data.

### Examples

The following examples exemplify illustrative examples of compounds of the present invention and are not to be construed as limiting the scope of the disclosure. Alternative mechanistic pathways and analogous structures within the scope of the invention may be apparent to those skilled in the art.

### General Methods

The starting materials and reagents used in preparing compounds described are either available from commercial suppliers such as Aldrich Chemical Co. (Wisconsin, USA) and Acros Organics Co. (New Jersey, USA) or were prepared using methods well-known to those skilled in the art of organic synthesis. All commercially purchased solvents and reagents were used as received. LCMS analysis was performed using an Applied Biosystems API-100 mass spectrometer equipped with a Shimadzu SCL-10A LC column: Altech platinum C18, 3 um, 33 mm×7 mm ID; gradient flow: 0 minutes, 10% CH₃CN; 5 minutes, 95% CH₃CN; 7 minutes, 95% CH₃CN; 7.5 minutes, 10% CH₃CN; 9 minutes, stop. Flash column chromatography was performed using Selecto Scientific flash silica gel, 32-63 mesh. Analytical and preparative TLC was performed using Analtech Silica gel GF plates. Chiral HPLC was performed using a Varian PrepStar system equipped with a Chiralpak OD column (Chiral Technologies).

### Example 1

### Preparation of Intermediate Compound A

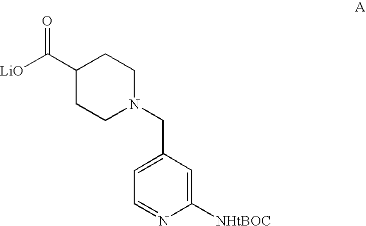

### Step 1—Synthesis of Compound A1

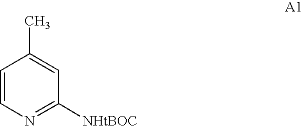

To a solution of 2-amino-4-methylpyridine (10.81 g, 100 mmol) in tert-butanol (250 mL) was added t-BOC anhydride (26.19 g, 120 mmol). The reaction mixture was stirred at 23° C. for about 15 hours, and then concentrated in vacuo. The crude oil obtained was dry loaded onto a silica gel column and flash chromatographed (eluant: 30% hexanes-CH₂Cl₂ to 0-2% acetone-CH₂Cl₂) to provide 15.25 g (73.32 mmol; 73%) of compound A1 as a white solid.

### Step 2—Synthesis of Compound A2

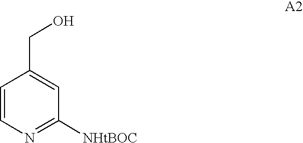

To a solution of compound A1 (35.96 g, 173 mmol) in TI-IF (1.41) at −78° C. was added n-BuLi (1.4 M in hexanes, 272 ml, 381 mmol) portionwise over 30 minutes. The reaction mixture was then allowed to warm slowly and was stirred for 2 h at 23° C., which resulted in the formation of an orange precipitate. The mixture was then cooled back to −78° C., and pre-dried oxygen (passed through a Drierite column) was bubbled through the suspension for 6 h while the temperature was maintained at −78° C. The color of the reaction mixture changed from orange to yellow during this time. The reaction was quenched at −78° C. with (CH₃)₂S (51.4 ml, 700 mmol) followed by AcOH (22 ml, 384 mmol) and allowed to warm to 23° C. After stirring for an additional 48 h, water was added and the product extracted into EtOAc. Purification by silica gel flash chromatography (eluant: 0-15% acetone/CH₂Cl₂) provided 20.15 g (90 mmol; 52%) of compound A2 as a pale yellow solid.

### Step 3—Synthesis of Compound A3

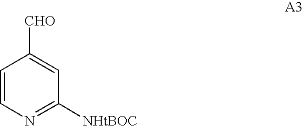

To a solution of compound A2 (19.15 g, 85.5 mmol) in CH₂Cl₂ (640 mL) was added a saturated aqueous solution of NaHCO₃ (8.62 g, 103 mmol) and NaBr (444 mg, 4.3 mmol). The reaction mixture was cooled to 0° C., and TEMPO (140 mg, 0.90 mmol) was added. Upon vigorous stirring, commercial bleach solution (122 ml, 0.7 M, 85.4 mmol) (5.25% in NaOCl) was added portionwise over 40 minutes. After an additional 20 min at 0° C., the reaction mixture was quenched with saturated aqueous Na₂S₂O₃ and allowed to warm to 23° C. Dilution with water and extraction with CH₂Cl₂, followed by concentration in vacuo and flash chromatography (eluant: 30% hexanes-CH₂Cl₂ to 0-2% acetone-CH₂Cl₂) provided 15.97 g (71.9 mmol; 84% yield) of compound A3 as an off-white solid.

### Step 4—Synthesis of Compound A4

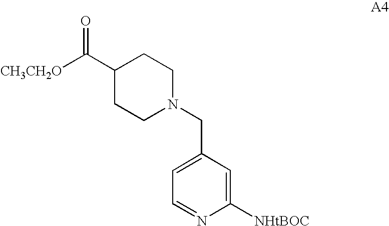

To a solution of compound A3 (11.87 g, 53.5 mmol) in CH₂Cl₂ (370 mL) was added ethyl isonipecotate (9.07 ml, 58.8 mmol) followed by four drops of AcOH. The reaction mixture was then stirred for 40 min at 23° C., after which NaB(OAc)₃H (22.68 g, 107 mmol) was added. The reaction mixture was stirred for about 15 hours at 23° C., neutralized with saturated aqueous NaHCO₃, diluted with water and extracted with CH₂Cl₂. Concentration of the organic extracts in vacuo, followed by silica gel flash chromatography (eluant: 0-4% sat. NH₃ in CH₃OH—CH₂Cl₂) provided 19.09 g (52.6 mmol; 98%) of compound A4 as an off-white solid.

### Step 5—Synthesis of Intermediate Compound A

To a solution of compound A4 (1.57 g, 4.33 mmol) in THF-water-CH₃OH (10 ml of a 3:1:1 mixture) was added LiOH monohydrate (0.125 g, 5.21 mmol). The reaction mixture was stirred for about 15 hours at 23° C., then concentrated in vacuo to provide 1.59 g of compound A as a yellowish solid which was used without further purification.

### Example 2

### Preparation of Intermediate Compound B

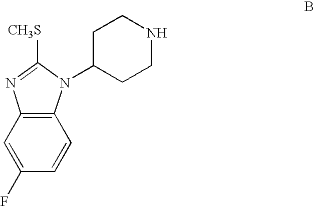

### Step 1—Synthesis of Compound B2

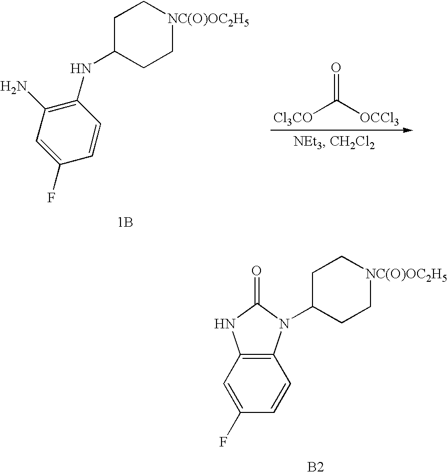

A solution of diamine 1B (see Example 1, Step 1) (20 g, 71.1 mmol) and Et₃N (30 ml, 213 mmol) in CH₂Cl₂ (400 mL), was cooled to 0° C. in an ice-water bath with stirring. To the stirred solution was added triphosgene (14.2 g, 47.3 mmol) cautiously (exotherm!) and portionwise over a period of 30 minutes. When addition was complete, stirring was continued at 0° C. for 1 h, then at room temperature for 16 hours. The mixture was washed with 0.5N NaOH (200 mL), the organic layer was dried over anhydrous MgSO₄ and concentrated in vacuo. Hot EtOAc (200 mL) was added to the semi-solid residue, and the resultant mixture was cooled to room temperature. Filtration yielded compound B2 as a white solid (16.5 g); and silica gel flash chromatography [CH₂Cl₂—CH₃OH (2N NH₃)=40:1] of the filtrate provided additional product as a white solid (2.7 g) [combined yield: 88%]. FABMS: 308 (MH⁺; 100%).

### Step 2—Synthesis of Compound B3

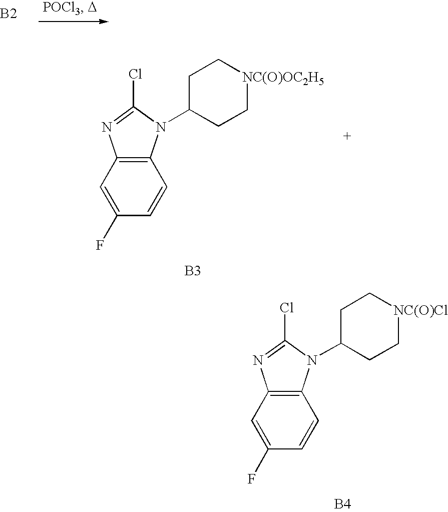

POCl₃ (100 mL) was added to compound B2 (17.2 g; 56 mmol) in a round-bottomed flask flushed with dry N₂. The mixture was placed in an oil bath heated to 108° C. and was maintained at reflux for 6 hours. POCl₃ was then removed in vacuo. The residue obtained was adjusted to pH˜9-10 with 7N methanolic ammonia and the resulting solution was concentrated in vacuo. CH₂Cl₂ was added to the residue, insoluble material was filtered off, and the filtrate was again concentrated in vacuo. The residue was crystallized from EtOH to provide compound B3 as a white solid (12.6 g; 67%). ES-MS: 326.1 (MH⁺; 100%).

Varying amounts of compound B4 may be formed in this process and can be converted to desired product B3 by careful in situ treatment in CH₂Cl₂ solution at 0° C. with one equivalent each of EtOH and NaH, followed by workup with ice-water and CH₂Cl₂.

### Step 3—Synthesis of Compound B4

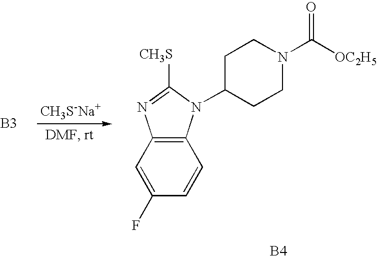

Sodium thiomethoxide (1.05 g; 15.0 mmol) was added to DMF (15 mL) in a round-bottomed flask flushed with N₂. After stirring at room temperature for 30 min, compound B3 (3.25 g, 10 mmol) was added, and the resultant mixture was allowed to stir at room temperature for 16 hours. EtOAc (100 mL) and water (50 mL) were then added to the reaction mixture and the aqueous layer was separated and further extracted with EtOAc (50 mL). The combined organic extracts were dried over anhydrous MgSO₄ and concentrated in vacuo. The residue obtained was purified via flash chromatography on silica gel, eluting with EtOAc-hexanes (3:4), to provide compound B4 as a white solid (2.12 g; 63%). FABMS: 338.3 (MH⁺; 100%).

### Step 4—Synthesis of Compound B

Trimethylsilyl iodide (1.77 ml; 12.5 mmol) was added to a solution of B4 (2.10 g; 6.23 mmol) in CHCl₃ (15 mL) under N₂, and the resultant solution was stirred at 55° C. for 7 hours. The reaction was quenched with EtOH (2 mL), and the mixture was concentrated in vacuo. The residue was precipitated from EtOH solution with Et₂O to provide compound B (hydriodide salt) as a pale yellow solid (1.61 g; 67%) which was used without further purification. ES-MS: 266.1 (MH⁺; 100%)

### Example 3

### Preparation of Intermediate Compound C

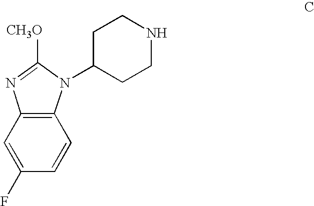

### Step 1—Synthesis of Compound C1

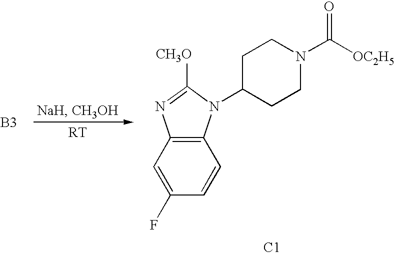

NaH (60 mg of a 60% dispersion; 1.48 mmol) was added to CH₃OH (4 mL) in a flask charged with N₂. After stirring at room temperature for 30 min, compound B3 (400 mg, 1.23 mmol) was added, and the resultant mixture was stirred at room temperature for 16 hours. CH₃OH was removed in vacuo, and to the residue obtained was added CH₂Cl₂ (30 mL) and water (10 mL). The organic layer was dried over anhydrous MgSO₄, filtered, and concentrated in vacuo. The residue obtained was purified using flash chromatography on silica gel, eluting with EtOAc-hexanes (3:2) to provide compound C1 as a white foam (0.232 g; 59%). ES-MS: 322.1 (MH⁺; 100%).

### Step 2—Synthesis of Compound C

1N aqueous KOH (4.82 mL; 4.82 mmol) was added to a solution of compound C1 in EtOH (15 mL), and the resultant mixture was stirred at 80° C. for 48 hours. The mixture was concentrated in vacuo and water (3 mL) and CH₂Cl₂ (15 mL) were added to the resulting residue. The organic layer was dried over anhydrous MgSO₄, then concentrated in vacuo to provide compound C as a colorless glass (160 mg; 95%). FABMS: 250.2 (MH⁺; 100%).

### Example 4

### Preparation of Intermediate Compound D

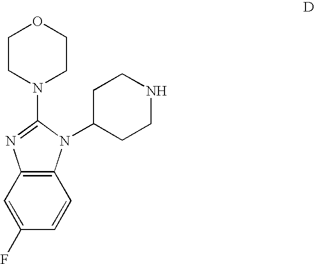

### Step 1—Synthesis of Compound D1

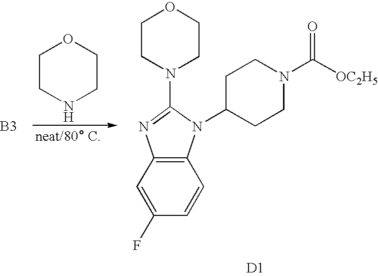

Compound B3 (300 mg; 0.923 mmol) and morpholine (3 mL) were mixed in a round-bottomed flask under N₂, and the resultant mixture was heated to 80° C. for 16 hours. Morpholine was removed in vacuo, and the residue obtained was dissolved in CH₂Cl₂ (20 mL). An insoluble white precipitate was filtered off, and the filtrate was concentrated in vacuo and purified using flash chromatography on silica gel, eluting with CH₂Cl₂-2N methanolic ammonia (45:1), to provide compound D1 as a colorless glass (0.325 g; 94%). ES-MS: 377.1 (MH⁺; 100%).

### Step 2—Synthesis of Compound D

Trimethylsilyl iodide (240 microliters; 1.64 mmol) was added to a solution of compound D1 (316 mg; 0.843 mmol) in CHCl₃ (2 mL) under N₂, and the resultant solution was stirred at 55° C. for 7 hours. The reaction was quenched with EtOH (2 mL), and the mixture was concentrated in vacuo. The residue obtained was basified to pH˜10 using a 1:1 (v/v) mixture of concentrated NH₄OH and water and the basic solution was then extracted with CH₂Cl₂ (2×5 mL). The combined organic extracts were dried over anhydrous MgSO₄ and concentrated in vacuo. The residue obtained was purified using flash chromatography on silica gel, eluting with CH₂Cl₂-2N methanolic ammonia (13:1), to provide compound D as a colorless glass. (181 mg; 70%). ES-MS: 305.1 (MH⁺; 100%).

### Example 5

### Preparation of Intermediate Compound E

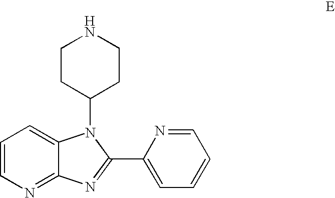

### Step 1—Synthesis of Compound E3

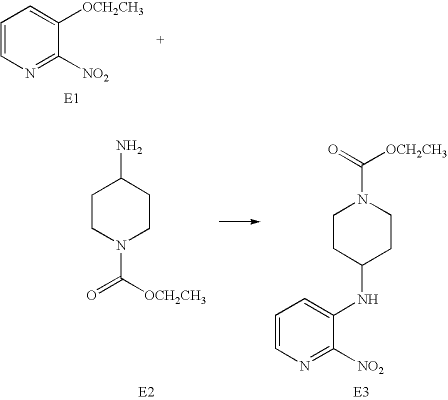

A solution of compound E1 (3.5 g, 21 mmol) and compound E2 (6.5 g, 38 mmol) in CH₂Cl₂ (3 mL) was heated to 110° C. for 24 h and room temperature for 24 hours. The reaction was diluted with CH₂Cl₂, washed with water and brine, dried (Na₂SO₄), and concentrated in vacuo. Purification of the resulting residue on a flash column (SiO₂, 40% to 60% EtOAc in hexanes) provided compound E3 (1.3 g, 21%; M+H=295).

### Step 2—Synthesis of Compound E4

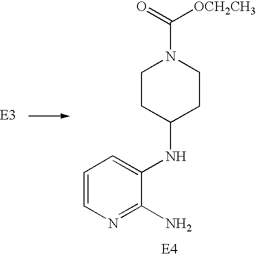

To a solution of compound E3 (1.3 g, 4.4 mmol) in CH₃OH (30 mL) was added Ra—Ni (0.5 g) and the mixture was hydrogenated under a H₂ atmosphere (50 psi) for 18 hours. Filtration through a pad of celite provided compound E4 as a grey solid that was used without further purification (1.05 g, 90%; M+H=265).

### Step 3—Synthesis of Compound E6

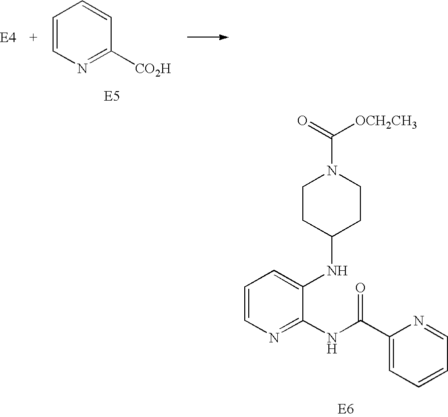

A solution of compound E4 (1.05 g, 3.97 mmol), compound E5 (0.49 g, 3.97 mmol), DEC (1.14 g, 5.96 mmol) and HOBT (0.8 g, 5.96 mmol) in CH₂Cl₂ (10 mL) was stirred for 18 h at room temperature. The reaction mixture was then diluted with additional CH₂Cl₂, washed with 5% aqueous NaOH and brine, then dried (Na₂SO₄) and concentrated in vacuo. Purification using flash chromatography (SiO, 8% EtOAc in hexane to 10% CH₃OH in EtOAc) provided compound E6 (0.35 g, 24%; M+H=370).

### Step 4—Synthesis of Compound E7

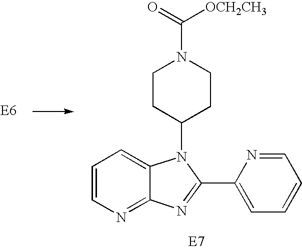

Compound E6 (0.7 g, 1.89 mmol) was dissolved in HOAc (10 mL) and heated to 120° C. for 3.5 hours. The reaction was cooled to room temperature, concentrated in vacuo, neutralized by the addition of 10% aqueous NaOH and extracted with CH₂Cl₂. The combined organic layers were dried (Na₂SO₄) and concentrated in vacuo to provide compound E7 (0.58 g, 87%; M+H=352) which was used in the next step without further purification.

### Step 5—Synthesis of Compound E

A solution of compound E7 (0.58 g, 1.65 mmol) and NaOH (0.43 g, 13.2 mmol) in EtOH/H₂O (9/1, 10 mL) was heated to 100° C. and allowed to stir at this temperature for 18 hours. The reaction was cooled and concentrated in vacuo and the residue obtained was purified using flash column chromatography (SiO₂, 10% CH₃OH saturated with ammonia in CH₂Cl₂) to provide compound E (0.42 g, 91%; M+H=280).

### Example 6

### Preparation of Intermediate Compound F

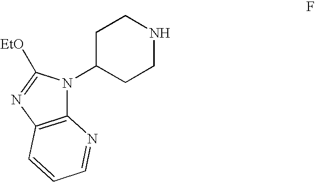

### Step 1—Synthesis of Compound F2

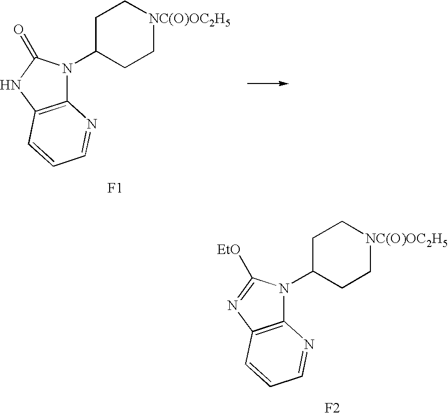

A solution of compound F1 (prepared by procedures analogous to P2-1) (10.5 g, 36.2 mmol) and 2,6-di-tert-butylpyridine (12.2 ml, 54.4 mmol) in CH₂Cl₂ (400 mL) was treated with Et₃O⁺BF₄⁻ (1M solution in CH₂Cl₂, 55 ml, 55 mmol). The reaction mixture was stirred at room temperature for 2 h, quenched with 1N NaOH (100 mL), extracted with CH₂Cl₂ (3×), dried with Na₂SO₄ and concentrated in vacuo. Purification using silica gel chromatography (eluant: 5-10% acetone/CH₂Cl₂) provided 6.37 g of compound F2 (20.0 mmol, 55%).

### Step 2—Synthesis of Compound F

Using the method described in Example 3, Step 2, compound F2 was converted to compound F.

### Example 7

### Preparation of Intermediate Compound G

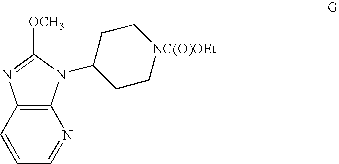

### Step 1—Synthesis of Compound G2

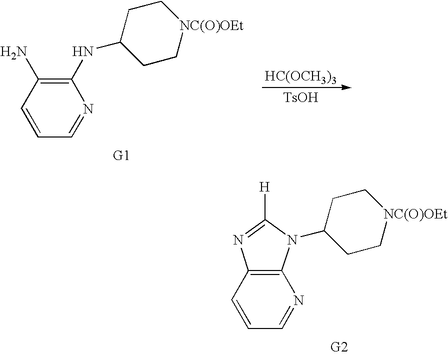

A mixture of compound G1 (40 g, 150 mmol), trimethyl orthoformate (66 ml, 64.0 g, 600 mmol) and a catalytic amount of p-toluenesulfonic acid monohydrate (300 mg, 1.58 mmol) was stirred under N₂ at 120° C. for 3 hours. Excess orthoformate was removed in vacuo and the resulting residue was partitioned between EtOAc (200 mL) and 1N NaOH (100 mL). The organic layer was washed with brine (100 mL) and dried over anhydrous MgSO₄. Drying agent was removed by filtration, and the filtrate was concentrated in vacuo. The residue was purified using silica gel flash chromatography (CH₂Cl₂/CH₃OH (2N NH₃)=45:1) to provide compound G2 as a dark purple syrup (27.2 g, 66%), which solidified upon standing. ES-MS: 275 (MH⁺; 100%).

### Step 2—Synthesis of Compound G3

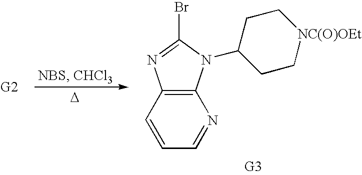

NBS was added portionwise (exotherm) to a solution of compound G2 (27 g, 100 mmol) in CHCl₃ (300 mL), and the resulting solution was stirred at 60° C. for 16 hours. Solvent was then removed in vacuo, and the residue obtained was partitioned between EtOAc (200 mL) and 0.7N Na₂S₂O₄ (250 mL). The organic layer was washed with brine (150 mL) and dried over anhydrous MgSO₄. Drying agent was removed by filtration, and the filtrate was concentrated in vacuo. The residue obtained was purified using silica gel flash chromatography [CH₂Cl₂/acetone=45:1] to provide compound G3 as a yellow solid (24.2 g, 69%). ES-MS: 353 (MH⁺; 100%).

### Step 3—Synthesis of Compound G

NaH (544 mg of a 60% dispersion, 13.6 mmol) was added to a solution of CH₃OH (0.551 ml, 436 mg, 13.6 mmol) in DMF (5 mL). The resultant mixture was stirred at room temperature for 30 min before adding compound G3 (3.99 g, 11.3 mmol). The reaction was stirred at room temperature for 16 h, then the reaction mixture was then partitioned between EtOAc (800 mL) and water (40 mL). The aqueous layer was extracted with EtOAc (40 mL). Combined organic extracts were washed with brine (30 mL) and dried over anhydrous MgSO₄. Drying agent was removed by filtration, and the filtrate was concentrated in vacuo to provide compound G as a white syrup (2.81 g, 81%), which was used without further purification. ES-MS: 305 (MH⁺; 100%).

### Example 8

### Preparation of Intermediate Compound H

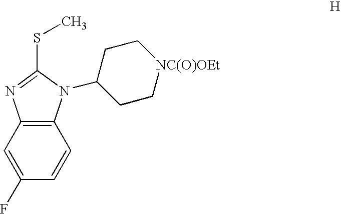

### Step 1—Synthesis of Compound HI

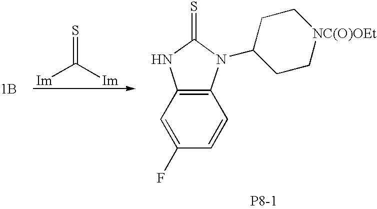

A solution of compound 1B (15 g, 52.8 mmol) and 1,1′-thiocarbonyldiimidazole (25 g, 140 mmol) in THF (300 mL) was stirred at 72° C. under N₂ for 16 h, during which time a precipitate formed. THF was removed in vacuo, and the residue obtained was purified by silica gel flash chromatography (CH₂Cl₂/acetone=20:1) to provide compound H1 as a light yellow solid (16.7 g, >95%). ES-MS: 324 (MH⁺; 100%).

### Step 2—Synthesis of Compound H

To a stirred mixture of compound H1 (4.00 g, 12.5 mmol) and K₂CO₃ (2.05 g, 13.6 mmol) in DMF (40 mL) under a N₂ atmosphere was added CH₃I (0.85 ml, 1.94 g, 13.6 mmol). The resultant mixture was stirred at room temperature for 16 h before partitioning between EtOAc (100 mL) and water (40 mL). The aqueous layer was extracted with EtOAc (40 mL). Combined extracts were washed with brine (30 mL) and dried over anhydrous MgSO₄. Drying agent was removed by filtration, and the filtrate was concentrated in vacuo to provide compound H as a foamy white solid (4.20 g, >95%), which was used without further purification. ES-MS: 338 (MH⁺; 100%).

### Example 9

### Preparation of Intermediate Compound J

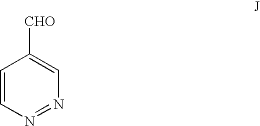

### Step 1—Synthesis of Compound J3

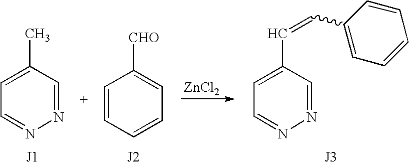

A solution of compound J1 (4.5 g, 47.8 mmoles), compound J2 (8.12 g, 76.5 mmoles), and anhydrous ZnCl₂ was heated to 160° C. under N₂, and allowed to stir at this temperature for 5 hours. The resulting oil was purified using flash chromatography on silica gel using 30% Hexanes/EtOAc to provide 5.92 grams (67%) of compound J3.

### Step 2—Synthesis of Compound J

OsO₄ (5.0 ml in t-butanol, 2.5% w/w) was added to compound J3 (5.9 g, 32.38 mmoles) dissolved in p-dioxane (87 mL) and water (29 mL). NaIO₄ (14.1 g, 65.92 mmoles) was added, with good stirring, in small portions, over a period of 6 hours. The mixture was then diluted with p-dioxane and filtered. After removing most of the solvent under reduced pressure, the residue was taken in CH₂Cl₂ (600 mL) and dried over anhydrous Na₂SO₄. After removal of the solvent, the mixture was purified using flash chromatography on silica gel using 5% CH₃OH/CH₂Cl₂ as eluent to provide compound J. Yield: 2.89 g (82%).

### Example 10

### Preparation of Intermediate Compound K

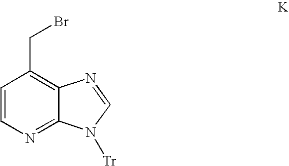

### Step 1—Synthesis of Compound K2

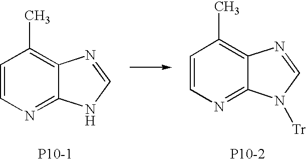

A solution of compound K1 (2 g, 15 mmol) in CH₂Cl₂ (50 mL) was treated with Et₃N (3 g, 30 mmol) and triphenylmethyl chloride (TrCl, 4.25 g, 15.3 mmol) and stirred at room temperature for about 15 hours. The solvent was removed in vacuo and the resulting residue purified using flash column chromatography (SiO₂, 20% EtOAc in hexane) to provide compound K2 (5.2 g, 46%).

### Step 2—Synthesis of Compound K

A solution of compound K2 (5.2 g, 14.6 mmol) in CCl₄ (80 mL) was treated with NBS (7.8 g, 43 mmol) and the reaction heated to 80° C. for about 15 hours. The reaction was cooled, filtered and concentrated in vacuo, and the resulting residue was purified using flash column chromatography (SiO₂, 20% to 30% EtOAc in hexane) to provide compound K (2.8 g, 42%, M+H=453, 455)

### Example 11

### Preparation of Intermediate Compound L

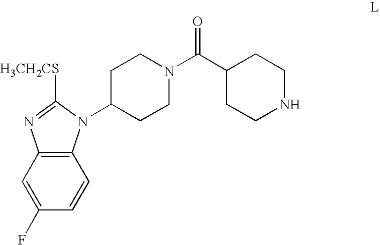

### Step 1—Synthesis of Compound L1

To a stirred solution of compound H1 (6.5 g, 20.1 mmol) in EtOH (80 mL) was added 25% (w/w) aqueous NaOH solution (20 mL). The resultant mixture was stirred at 90° C. for 16 hours. EtOH was removed under vacuum, and the residue was adsorbed directly onto silica gel and subjected to flash chromatography (CH₂Cl₂/2N methanolic ammonia=9:1) to obtain compound L1 as a white solid (4.46 g, 70%). ES-MS: 252 (MH⁺; 100%).

### Step 2—Synthesis of Compound L2

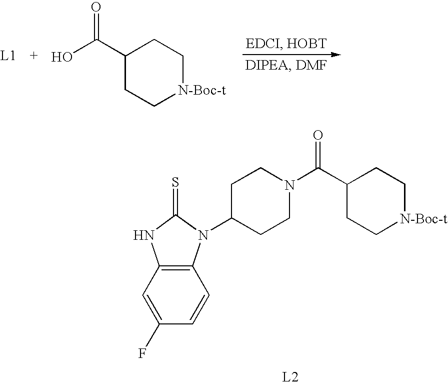

A mixture of compound L1 (3.95 g; 15.7 mmol), BOC-isonipecotic acid (3.60 g; 15.7 mmol), HOBT (3.19 g; 23.6 mmol), DIPEA (3 ml; 2.23 g; 17.2 mmol) and EDCI (4.50 g; 23.6 mmol) in DMF (30 mL) was stirred under N₂ at room temperature for 16 hours. The reaction mixture was partitioned between EtOAc (60 mL) and water (40 mL). The aqueous phase was extracted with EtOAc (40 mL), and the combined extracts were washed with brine (40 mL) and dried over anhydrous MgSO₄. Drying agent was removed by filtration, and the filtrate was concentrated in vacuo. The resulting residue was purified using silica gel flash chromatography (CH₂Cl₂/CH₃OH(2N NH₃)=40:1) to provide compound L2 as a white solid (˜7.3 g, ˜100%), which was used without further purification. ES-MS: 463 (MH⁺; 70%); 407 (100%).

### Step 3—Synthesis of Compound L3

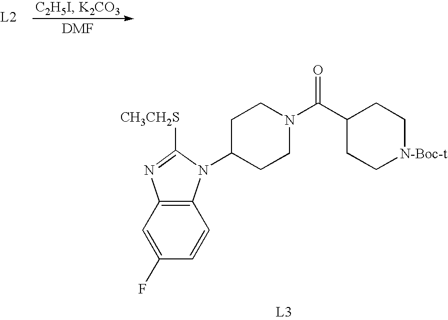

To a stirred mixture of compound L2 (460 mg; 1 mmol) and K₂CO₃ (165 mg; 1.20 mmol) in DMF (4 mL) under a N₂ atmosphere was added EtI (92 microliters; 179 mg; 1.15 mmol). The resultant mixture was stirred at room temperature for 16 h and was then partitioned between EtOAc (20 mL) and water (10 mL). The aqueous phase was extracted with EtOAc (10 mL), and the combined extracts were washed with brine (20 mL) and dried over anhydrous MgSO₄. Drying agent was removed by filtration, and the filtrate was concentrated in vacuo to provide compound L3 as a pale yellow foam (471 mg, 96%) which was used without further purification. ES-MS: 463 (MH⁺; 85%); 435 (100%).

### Step 4—Synthesis of Compound L

To a solution of compound L3 (465 mg; 0.949 mmol) in CH₂Cl₂ (4 mL) was added TFA (1 ml; 1.54 g; 13.5 mmol). The resultant solution was stirred for 2 h at room temperature and was then partitioned between CH₂Cl₂ (20 mL) and 1:1 (v/v) concentrated NH₄OH:water (5 mL). The aqueous phase was extracted successively with 95:5 CH₂Cl₂:EtOH (5 mL) and EtOAc (5 mL). The combined extracts were dried over anhydrous MgSO₄. Drying agent was removed by filtration, and the filtrate was concentrated in vacuo to provide compound L as a pale white foam (353 mg, 95%) which was used without further purification. ES-MS: 391 (MH⁺; 100%).

### Example 12

### Preparation of Compound 1

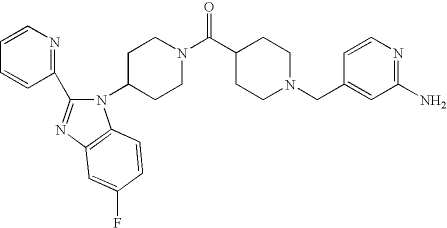

### Step 1—Synthesis of Compound 1C

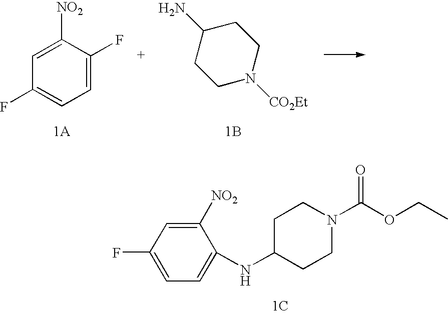

A mixture of compound 1A (25 g, 0.16 mol), compound 1B (27 g, 0.16 mol), K₂CO₃ (26 g, 0.19 mol), and NO (2.4 g, 0.016 mol) in dimethylacetamide (50 mL) was heated at 140° C. for 3.5 hours. The reaction mixture was concentrated to one-third volume, poured onto saturated aqueous NaHCO₃, and extracted with EtOAc (4×). The combined organic layers were washed with water (2×) and brine, dried over Na₂SO₄, and concentrated in vacuo. Recrystallization of the resulting residue from EtOH provided compound 1C (48 g, 98%).

### Step 2—Synthesis of Compound 1D

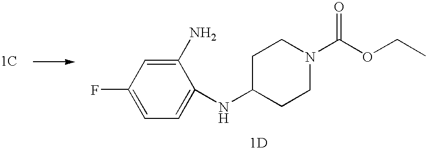

A suspension of compound 1C (20.00 g, 64.2 mmol,) and Raney® 2800 Nickel (5.0 g) in ethanol (70 mL) and THF (140 mL) was shaken under H₂ (40 psi) for 2 hours. The mixture was filtered through a short pad packed with celite. The filtrate was concentrated in vacuo and dried under vacuum to provide compound 1D as a tan solid (18.20 g, ˜100%).

### Step 3—Synthesis of Compound 1E

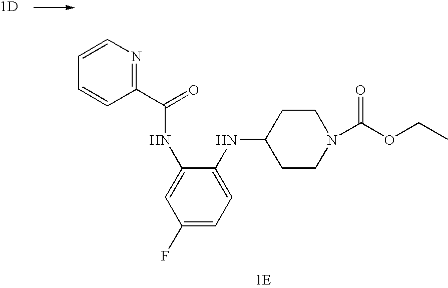

A solution of compound 1D (5.00 g, 17.77 mmol) and picolinoyl chloride hydrochloride (3.16 g, 17.75 mmol) in CH₂Cl₂ (400 mL) and Et₃N (15 mL) was stirred at room temperature. After 15 h, the reaction was diluted with CH₂Cl₂, washed with water, dried over Na₂SO₄, concentrated in vacuo, and dried on vacuum to provide compound 1E as a brown foam (6.47 g, 94%).

### Step 4—Synthesis of Compound 1F

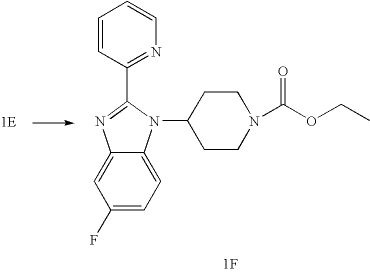

A solution of compound 1E (1.77 g, 4.58 mmol) in ethanol (50 mL) and concentrated H₂SO₄ (5.0 mL) was refluxed for 3 h, cooled to RT, then neutralized to pH 10 using 1.0 M aqueous NaOH. The resulting mixture was extracted with CH₂Cl₂ and the combined organic solutions were dried over Na₂SO₄ and concentrated in vacuo. The residue obtained was purified using flash chromatography (silica gel, 5% CH₃OH in CH₂Cl₂ as an eluent) to provide compound 1F as a tan foam (1.58 g, 94%).

### Step 5—Synthesis of Compound 1G

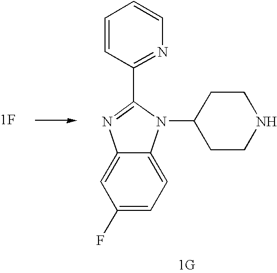

Iodotrimethylsilane (6.30 g, 31.48 mmol) was added to a solution of compound 1F (3.88 g, 10.53 mmol) in anhydrous 1,2-dichloroethane (40 mL). The resulting solution was stirred at 75° C. for 4 hours, cooled to room temperature, then treated with 1.0 M NaOH solution. The mixture was then extracted with CH₂Cl₂ and the combined extracts were washed with water, dried over Na₂SO₄, and concentrated in vacuo. Purification of the residue using flash chromatography (silica gel, 10% CH₃OH in CH₂Cl₂ as an eluent) provided compound 1G as an off-white foam (2.10 g, 67%).

### Step 6—Synthesis of Compound 1H

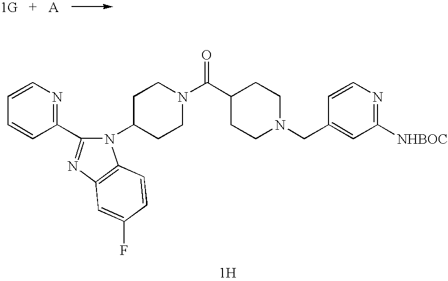

Compound 1E (5.80 g, 19.6 mmol) and compound A (Example 1, 5.32 g, 23.4 mmol) were dissolved in DMF (60 mL) and CH₂Cl₂ (60 mL). To the resulting solution was added sequentially, EDCI hydrochloride (5.70 g, 24.50 mmol), HOBT (1.30 g, 24.50 mmol), and diisopropylethylamine (5.08 g, 39.6 mmol). The resulting reaction mixture was stirred at 70° C. for 4 hours, cooled to RT, diluted with CH₂Cl₂, washed with water, dried over Na₂SO₄, and concentrated in vacuo. Flash chromatography (silica gel, 10% CH₃OH in CH₂Cl₂ as an eluent) of the resulting residue provided compound 1H as a tan foam (7.89 g, 65%).

### Step 7—Synthesis of Compound 1

A solution of compound 1H (7.89 g, 12.88 mmol) and TFA (29 g, 257 mmol) in CH₂Cl₂ (65 mL) was stirred at room temperature for 12 h, and was then neutralized with 1.0 M NaOH, and extracted with CH₂Cl₂. The combined organic layers were washed with water, dried over Na₂SO₄ and concentrated in vacuo. Purification of the resulting residue using flash chromatography provided compound 1 as a white solid (5.80 g, 88%). MS: 514 (MH⁺).

### Example 13

### Preparation of Compound 2

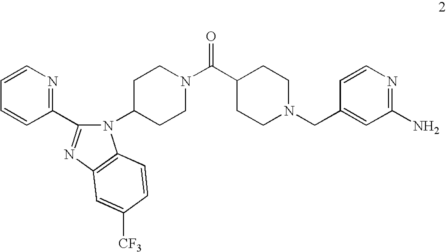

### Step 1—Synthesis of Compound 2B

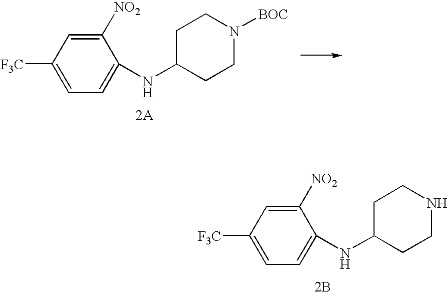

TFA (200 ml, 2.596 mol) was added to a solution of 2A (20 g, 51.36 mmol) in CH₂Cl₂ (100 mL). The resulting reaction mixture was stirred at room temperature for 6 h, neutralized with 1.0 M NaOH, and extracted. The combined extracts were washed with water, dried over Na₂SO₄, and concentrated in vacuo. Flash chromatography provided compound 2B as an orange solid (13.50 g, 91%).

### Step 2—Synthesis of Compound 2C

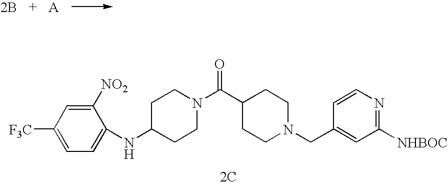

Amine 2B (1.50 g, 5.19 mmol) and compound A (Example 1, 1.75 g, 5.13 mmol) were dissolved in DMF (10 mL) and CH₂Cl₂ (10 mL). To the resulting solution was added sequentially, EDCI hydrochloride (1.50 g, 7.83 mmol), HOBT (1.05 g, 7.82 mmol), and diisopropylethylamine (3.71 g, 28.70 mmol). The resulting reaction mixture was stirred at 70° C. for 18 h, cooled to RT, diluted with CH₂Cl₂, washed with water, dried over Na₂SO₄, and concentrated in vacuo. Flash chromatography of the resulting residue provided compound 2C an orange gel (2.31 g, 74%).

### Step 3—Synthesis of Compound 2D

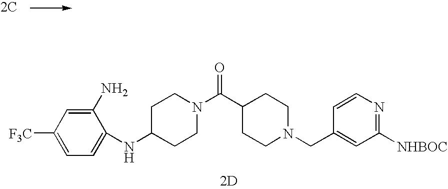

A suspension of compound 2C (2.10 g, 3.46 mmol,) and Raney® 2800 Nickel (1.0 g) in CH₃OH (100 mL) was shaken under H₂ (30 psi) for 6 hours. The mixture was filtered through a short pad of celite and the filtrate was concentrated in vacuo to provide compound 2D as an orange solid (1.80 g, 90%).

### Step 4—Synthesis of Compound 2E

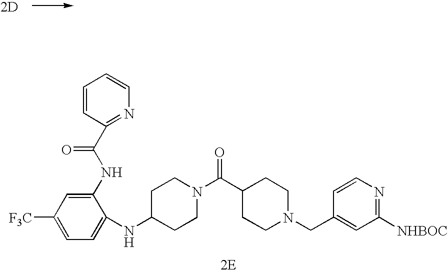

Amine 2D (200 mg, 0.347 mmol) and picolinoyl chloride hydrochloride (62 mg, 0.348 mmol) were dissolved in CH₂Cl₂. Et₃N was then introduced via a syringe. The resulting solution was stirred at room temperature for 6 h, treated with 1.0 M NaOH solution, and extracted. The extracts were washed with water, dried over Na₂SO₄, and concentrated in vacuo. Purification of the resulting residue using flash chromatography provided compound 2E as a white foam (167 mg, 71% yield).

### Step 5—Synthesis of Compound 2

A solution of compound 2E (160 mg, 0.235 mmol) and H₂SO₄ (concentrated, 0.50 mL) in ethanol (10 mL) was refluxed for 2.5 h, cooled to RT, and neutralized with 1.0 M NaOH. After extraction of the mixture, the combined organic layers were washed with water, dried over Na₂SO₄, and concentrated in vacuo. Flash chromatography of the crude residue provided compound 2 as a white solid (88 mg, 66%). MS: 564 (MH⁺)

### Example 14

### Preparation of Compound 3

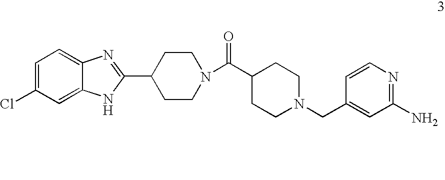

### Step 1—Synthesis of Compound 3C

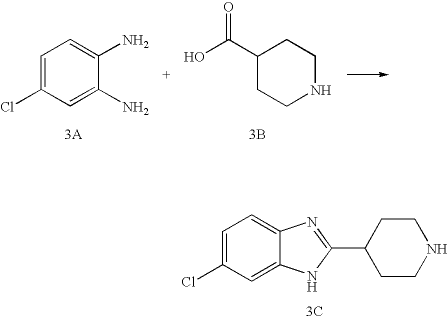

Compound 3A (1.43 g, 10 mmol) and isonipecotic acid 3B (1.29 g, 10 mmol) were taken up in PPA (20 g) and the resulting mixture was heated at 180° C. for 3.5 h, cooled to RT and diluted with water to 100 mL. The solution was then basified to pH 14 using solid NaOH. The resulting precipitate was then filtered off and washed repeatedly with CH₃OH. The combined CH₃OH extracts were concentrated in vacuo and the resulting residue was purified using flash chromatography on silica gel (25-40% 5N NH₃ in CH₃OH/CH₂Cl₂) to provide compound 3C as a dark solid (1.90 g, 81%).

### Step 2—Synthesis of Compound 3E

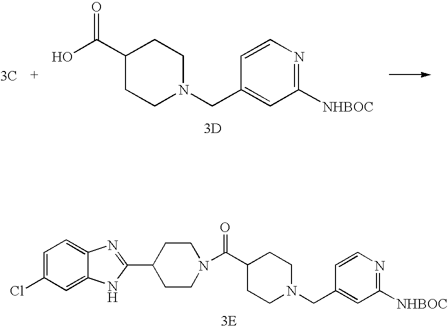

To the mixture of compound 3D (181 mg, 0.54 mmol), HATU (247 mg, 0.65 mmol) and Et₃N (84 μl, 0.6 mmol) in DMF (12 mL) was added compound 3C (126 mg, 0.54 mmol). The resulting mixture was stirred at room temperature for 24 h, concentrated in vacuo. The resulting residue was then dissolved in CH₃OH and the resulting solution was concentrated in vacuo. The resulting residue was purified using flash chromatography on silica gel (5-10% 5N NH₃ in CH₃OH/CH₂Cl₂) to provide compound 3E as a yellow oil (210 mg, 70%).

### Step 3—Synthesis of Compound 3

A solution of compound 3E (96 mg, 0.174 mmol) in 15 ml of 1M HCl in 25% CH₃OH/dioxane was stirred at room temperature for 48 hours. The mixture was concentrated in vacuo and the resulting residue was dried under high vacuum then dissolved in CH₃OH. The resulting solution was concentrated in vacuo and the resulting residue was purified using flash chromatography on silica gel (10-15% 5N NH₃ in CH₃OH/CH₂Cl₂) to provide the title compound 3 as a clear oil (48 mg, 61%). MS: 453 (MH⁺)

### Example 15

### Preparation of Compound 4

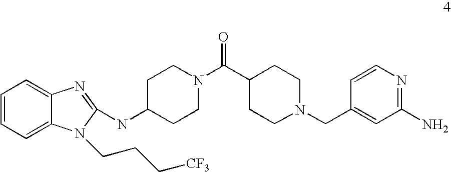

### Step 1—Synthesis of Compound 4C

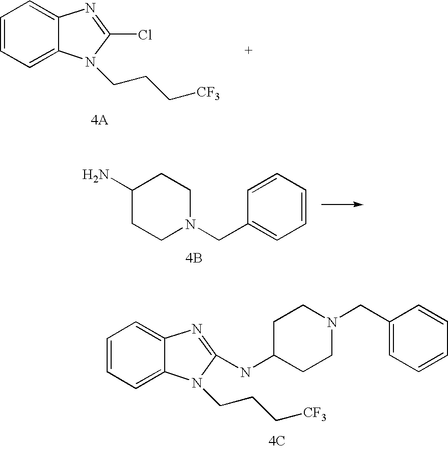

A mixture of compound 4A (1.75 g, 6.66 mmol) and compound 4B (2.93 g, 15.07 mmol) was stirred at 120° C. for 2 days, then cooled to RT. vThe reaction mixture was treated with 1.0 M NaOH solution (30 mL), then extracted with EtOAc. The combined organic layers were washed with water, dried over Na₂SO₄, then concentrated in vacuo. The resulting crude residue was purified using flash chromatography (silica gel, 50% EtOAc in hexanes as eluent) to provide 510 mg of compound 4C (18%).

### Step 2—Synthesis of Compound 4D

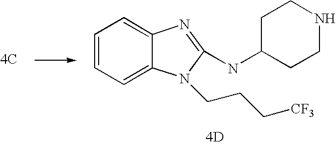

To a 500 mL pressured bottle was added a solution of compound 4C (490 mg, 1.18 mmol) in CH₃OH (20 mL). Under a N₂ stream, palladium hydroxide (300 mg, 20 wt. % on carbon) solid was added to the solution and the resulting reaction was shaken under 55 psi of hydrogen for 40 h, then filtered. The filtrate was concentrated in vacuo and the residue obtained was dried under vacuum to provide compound 4D as a yellow solid (340 mg, 88%).

### Step 3—Synthesis of Compound 4E

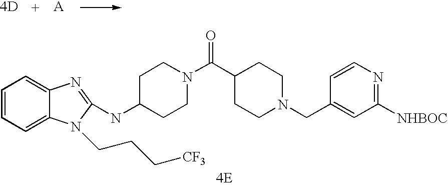

To a 50 mL round-bottomed flask were successively added compound 4D (287 mg, 0.88 mmol), compound A (Example 1, 300 mg, 0.88 mmol), EDCI hydrochloride (210 mg, 1.10 mmol), HOBT (149 mg, 1.10 mmol), and diisopropylethylamine (228 mg, 1.76 mmol). DMF (3 mL) and CH₂Cl₂ (3 mL). The resulting reaction mixture was stirred at 70° C. for 15 h and cooled to RT. After addition of 1 N NaHCO₃ solution, the resulting mixture was extracted with CH₂Cl₂. The combined organic extracts were dried over Na₂SO₄ and concentrated in vacuo. The resulting crude product was purified using flash chromatography on silica gel (10% CH₃OH in CH₂Cl₂ as eluent) to provide compound 4E as a solid (231 mg, 41%).

### Step 4—Synthesis of Compound 4

To a 25 ml round-bottomed flask was added a solution of compound 4E (200 mg, 0.31 mmol) in CH₂Cl₂ (2.5 mL). TFA was then added to the solution via a syringe. The resulting reaction was stirred at room temperature for 15 h, diluted with CH₂Cl₂, neutralized with 1.0 M NaOH solution, and separated. The organic solution was washed with water and dried over Na₂SO₄. After evaporation of the solvent, the crude product was purified using preparative TLC (10% CH₃OH in CH₂Cl₂ as the eluent) to provide compound 4 as a white solid (85 mg, 50%). MS: 544 (MH⁺).

### Example 16

### Preparation of Compound 5

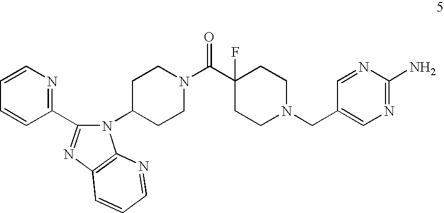

### Step 1—Synthesis of Compound 5B

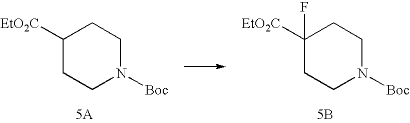

A solution of compound 5A (100 g, 0.389 mol) in THF (400 mL) was added dropwise over 1.0 h to a solution of LDA (233 mL, 2.0 M in THF/heptane/ethyl-benzene, 0.466 mol) in THF (300mL) at 0° C. The red-orange solution was stirred at 0° C. for 30 min, and then transferred by cannula to a pre-cooled (0° C.) solution of N-fluorobenzenesulfonimide (153 g, 0.485 mol) in dry THF (600 mL). The resulting reaction was stirred at 0° C. for 30 min, and then at 20° C. for 18 hours. The total solvent volume was reduced in vacuo to approximately one third of its original volume, then EtOAc (1 L) was added. The resulting solution was washed sequentially with water, 0.1 N aq. HCl, saturated aq. NaHCO₃, and brine. The organic layer was dried over MgSO₄, filtered, and concentrated in vacuo to provide a crude liquid which was purified using flash chromatography (6:1 hexanes-EtOAc) to provide compound 5B (93.5 g, 87%).

### Step 2—Synthesis of Compound 5C

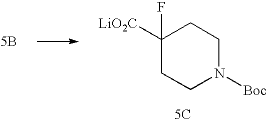

To a solution of compound 5B (50 g, 0.181 mol) in THF (300 mL) in CH₃OH (200 mL) was added a solution of LiOH—H₂O (9.2 g, 0.218 mol) in water (100 mL) and the resulting reaction was heated to 45° C. and allowed to stir at this temperature for 6 hours. The reaction mixture was then cooled to RT, concentrated in vacuo, and the resulting residue was dried in vacuo to provide compound 5C (45 g, 100%).

### Step 3—Synthesis of Compound 5D

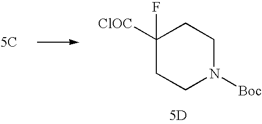

Compound 5C (20.4 g, 0.081 mol) was added slowly to a flask containing CH₂Cl₂ (250 mL) at 20° C. The resulting white slurry was cooled to 0° C. and treated slowly with oxalyl chloride (6.7 ml, 0.075 mol) and a drop of DMF. The reaction was allowed to stir at 20° C. for 0.5 h, then was concentrated in vacuo and the resulting residue dried in vacuo to provide compound 5D.

### Step 4—Synthesis of Compound 5F

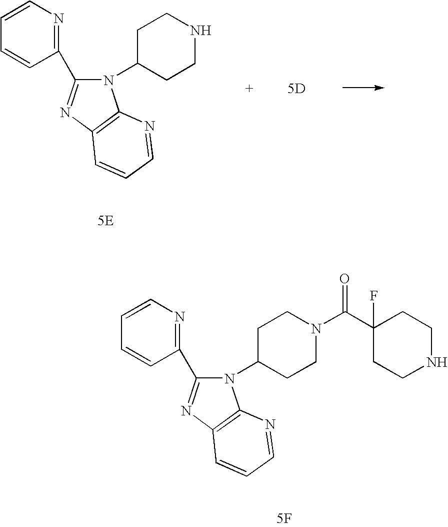

To a solution of compound 5D (0.075 mol) in CH₂Cl₂ (250 mL) was added a solution of compound 5E (15 g, 0.054 mol) in iPr₂NEt (25 ml, 0.135 mol) while maintaining a temperature of 20° C. After 1 h, the mixture was concentrated in vacuo and the resulting residue was taken up in CH₃OH (200 mL)/CH₂Cl₂ (200 mL)/H₂O (1 mL) and the resulting solution was allowed to stir for 1 h at 20° C. The solvent was then removed in vacuo and the resulting residue was treated with a solution of TFA (200 mL) in CH₂Cl₂ (250 mL) at 20° C. The resulting solution was purified using flash chromatography (0-7% 7N NH₃—CH₃OH/CH₂Cl₂) to provide compound 5F (80-90% yield from 5C).

### Step 5—Synthesis of Compound 5

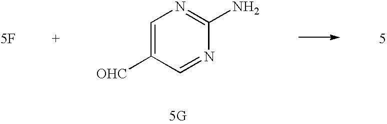

To a solution of compound 5F (0.41 g, 1.0 mmol) in CH₂Cl₂ (20 mL) was added a solution of compound 5G (0.31 g, 2.5 mmol, JP Patent 63227573, 1988), NaBH(OAc)₃ (0.53 g, 2.5 mmol) and few drops of AcOH and the resulting reaction was allowed to stir for about 15 hours at 20° C. The reaction mixture was partitioned between 10% NaOH and CH₂Cl₂ and the organic layer was dried with Na₂SO₄ then concentrated in vacuo. The resulting residue was purified using flash chromatography (0-5% 7N NH₃—CH₃OH/CH₂Cl₂) to provide compound 5 (0.45 g, 87%). MS: 516 (M+H).

### Example 17

### Preparation of Compound 6

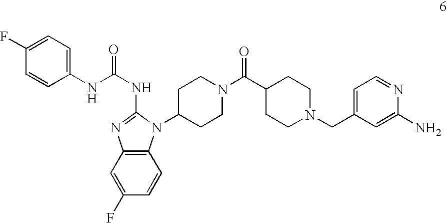

### Step 1—Synthesis of Compound 6A

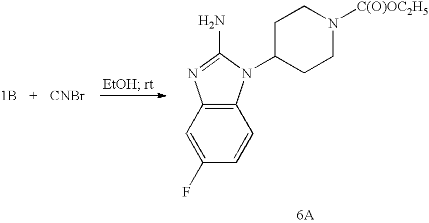

To a stirred solution of compound 1B (1.0 g, 3.55 mmol) in C₂H₅OH (25 mL), at room temperature was added portionwise solid CNBr (564 mg; 5.33 mmol). The resulting solution was allowed to stir at room temperature for 5 days before being concentrated in vacuo. The residual oil was partitioned between EtOAc (30 mL) and 2M Na₂CO₃ (10 mL), the aqueous layer was collected and adjusted to pH˜10 using 6N NaOH, and the basic solution was re-extracted using EtOAc (2×10 mL). The combined organic extracts were washed with brine (5 mL), then filtered through anhydrous MgSO₄. The filtrate was concentrated in vacuo to provide compound 6A as brown powder (1.03 g; 94%) which was used without further purification. FABMS: 307 (MH⁺; 100%).

### Step 2—Synthesis of Compound 6B

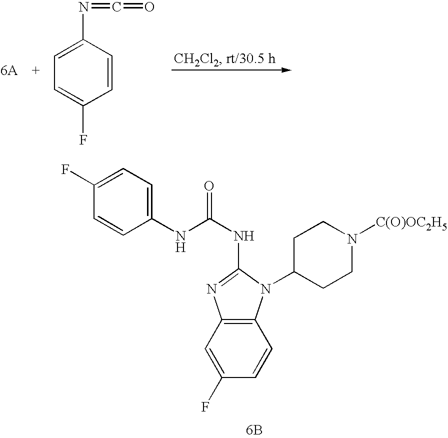

In a dry flask, under an inert atmosphere, a solution of compound 6A (369 mg; 1.20 mmol) in CH₂Cl₂ (11 mL) was stirred with sonication until the solution turned a clear amber color. To the amber solution was added 4-fluorophenyl isocyanate (158 μL; 190 mg; 1.38 mmol). The reaction was allowed to stir for 30.5 h at room temperature, then a few drops of CH₃OH were added to the reaction solution, and the reaction mixture was concentrated in vacuo. The residual solid was dissolved in refluxing Et₂O (˜30 mL). Insoluble matter was filtered, and the filtrate was diluted to a volume of ˜60 ml using warm hexanes. The solution was then concentrated on a steam bath to a volume of ˜30 ml, at which point precipitation was noticed. The resulting mixture was allowed to stand at room temperature for ˜3 h and was then filtered. The collected solid was washed with Et₂O-hexanes (1:1 v/v) and dried to provide compound 6B as a reddish-brown powder (394 mg; 74%) which was used without further purification. FABMS: 444 (MH⁺; 100%).

### Step 3—Synthesis of Compound 6C

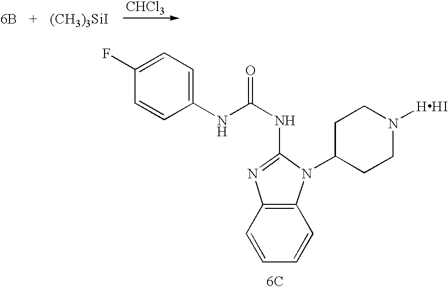

To a stirred suspension of compound 6B (333 mg; 0.751 mmol) in CHCl₃ (2 mL), was added (CH₃)₃SiI (214 μL; 301 mg; 1.51 mmol). The resulting reddish-brown solution was allowed to stir at room temperature for 20 min, then transferred to an oil bath that was preheated to 50° C. After stirring for 5 h at 50° C., a second portion of (CH₃)₃SiI (54 μL; 75 mg; 0.378 mmol) was added and stirring continued at 50° C. for an additional 2.5 hours. The reaction mixture (consisting of solid and solution phases) was then removed from the heating bath and CH₃OH (2.5 mL) was added to the reaction mixture in two portions. The resulting solution was stirred and warmed to 50° C. for a few minutes, allowed to cool to RT, then filtered. The collected solids were washed with 1:1 (v/v) CH₃OH-EtOAc to provide the hydriodide salt form of compound 6C as a pale reddish-brown powder (356 mg) which was used without further purification. FABMS: 372 (MH⁺; 100%).

### Step 4—Synthesis of Compound 6D

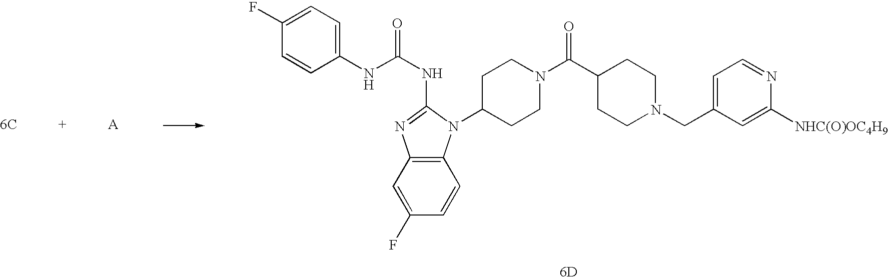

To a stirred suspension of compound 6C (340 mg; 0.681 mmol), compound A (Example 1, 228 mg; 0.681 mmol), HOBT (9.2 mg; 0.0681 mmol) and NEt₃ (379 microliters; 275 mg; 2.72 mmol) in DMF (13 mL) was added solid EDCI (163 mg; 0.851 mmol). The cloudy reaction mixture was placed in a oil bath (preheated to 50° C.) and the reaction was allowed to stir at 50° C. for 30 min, after which time the resultant clear, amber solution was allowed to stirred for an additional 23.5 h at room temperature. A few drops of water were added to the reaction mixture, and the reaction mixture was concentrated at 60° C. in vacuo. The concentrate was partitioned between EtOAc (20 mL) and water (5 mL)-brine (2.5 mL) and The aqueous phase was extracted with EtOAc (2×5 mL). Combined extracts were washed with brine (2.5 mL) and filtered through anhydrous MgSO₄. The filtrate was concentrated in vacuo, and the residue obtained was purified using flash chromatography on silica gel (gradient elution of CH₂Cl₂—CH₃OH—NH₄OH (97:3:0.5→96:4:0.5)) to provide compound 6D (222 mg; 47%) as pale yellow powder. FABMS: 689 (MH⁺; ˜93%); 578 (˜58%); 478 (100%).

### Step 5—Synthesis of Compound 6

To a solution of compound 6D (208 mg; 0.302 mmol) in CH₂Cl₂ (3 mL) was added TFA (928 μL; 1.37 g; 12.1 mmol) and the reaction flask was then flushed with dry N₂, sealed and allowed to stand at room temperature for 6 hours. The reaction mixture was then concentrated in vacuo and the residue obtained was partitioned between EtOAc (20 mL) and 2M Na₂CO₃ (3 mL) plus sufficient water to provide two clear phases. The aqueous phase was extracted with EtOAc (3×5 mL). The combined organic extracts were washed with brine (3 mL), then filtered through anhydrous MgSO₄. The filtrate was concentrated in vacuo and the residue obtained was purified using flash chromatography on silica gel (eluting with CH₂Cl₂—CH₃OH—NH₄OH (97:3:0.5)) to provide compound 6 as pale yellow powder (130 mg; 72%). FABMS: 589 (MH⁺; ˜64%); 478 (100%).

Using the methods described in Examples 1-17, compounds 7-387 were prepared:

| No. | R | R²⁵ | R³ | R¹³ | Z | R⁸ | Physical Data MS (MH⁺) |
|---|---|---|---|---|---|---|---|
| 7 | —CH₃ | 5-OCH₃ | H | H | —CH₂— | 2-NH₂ | 463 |
| 8 | —CH₃ | 6-Cl | H | H | —CH₂— | 2-NH₂ | 467 |
| 9 | —CH₃ | 5-Cl | H | H | —CH₂— | 2-NH₂ | 467 |
| 10 | —CH₃ | 5-Br | H | H | —CH₂— | 2-NH₂ | 512 |
| 11 | 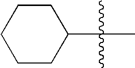 | 5-Cl | H | H | —CH₂— | 2-NH₂ | 535 |
| 12 | benzyl | 5-F | H | H | —CH₂— | 2-NH₂ | 527 |
| 13 | —CH(CH₃)₂ | 5-Br | H | H | —CH₂— | 2-NH₂ | 540 |
| 14 | —CH₂NH₂ | H | H | H | —CH₂— | 2-NH₂ | 488 |
| 15 | —CH₂NHSO₂CH₃ | H | H | H | —CH₂— | 2-NH₂ | 526 |
| 16 | —CH₂NHC(O)CH₃ | 5-Cl | H | H | —CH₂— | 2-NH₂ | |
| 17 | —CH₂OCH₃ | 5-F | H | H | —CH₂— | 2-NH₂ | 481 |
| 18 | —CH₂NH₂ | 5-Cl | H | H | —CH₂— | 2-NH₂ | 482 |
| 19 | —CH₂OCH₃ | 6,7-di-F | H | H | —CH₂— | 2-NH₂ | 499 |
| 20 | 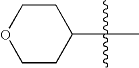 | 6-F | H | H | —CH₂— | 2-NH₂ | 521 |
| 21 | 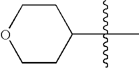 | 5-F | H | H | —CH₂— | 2-NH₂ | 521 |
| 22 | 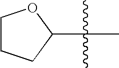 | 6-F | H | H | —CH₂— | 2-NH₂ | 507 |
| 23 | 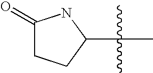 | 5-F | H | H | —CH₂— | 2-NH₂ | 520 |
| 24 | 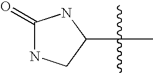 | 5-F | H | H | —CH₂— | 2-NH₂ | 521 |
| 25 | 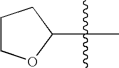 | 5-Br | H | H | —CH₂— | 2-NH₂ | 568 |
| 26 | 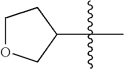 | 5-F | H | H | —CH₂— | 2-NH₂ | 507 |
| 27 | 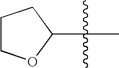 | 5-F | H | H | —CH₂— | 2-NH₂ | 507 |
| 28 | 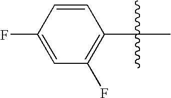 | H | H | H | —CH₂— | 2-NH₂ | 531 |
| 29 | 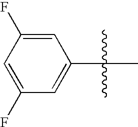 | 5-F | H | H | —CH₂— | 2-NH₂ | 549 |
| 30 | 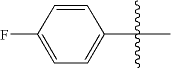 | 6-F | H | H | —CH₂— | 2-NH₂ | 531 |
| 31 | 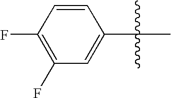 | 6,7-di-F | H | H | —CH₂— | 2-NH₂ | 567 |
| 32 | 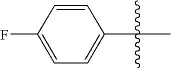 | 6-Cl | H | H | —CH₂— | 2-NH₂ | 547 |
| 33 | 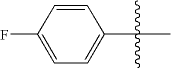 | 5-F | H | H | —CH₂— | 2-NH₂ | 531 |
| 34 | 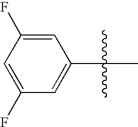 | 5-Cl | H | H | —CH₂— | 2-NH₂ | 565 |
| 35 | 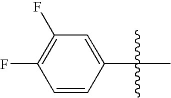 | H | H | H | —CH₂— | 2-NH₂ | 531 |
| 36 | 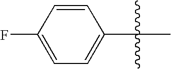 | 5-Cl | H | H | —CH₂— | 2-NH₂ | 547 |
| 37 | 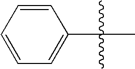 | 5-Cl | H | H | —CH₂— | 2-NH₂ | 529 |
| 38 | 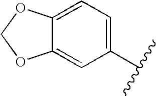 | 6-F | H | H | —CH₂— | 2-NH₂ | 557 |
| 39 | 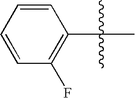 | 5-Br | H | H | —CH₂— | 2-NH₂ | 592 |
| 40 | 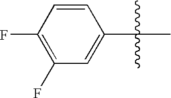 | 5-Br | H | H | —CH₂— | 2-NH₂ | 610 |
| 41 | 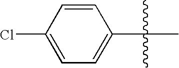 | 5-F | H | H | —CH₂— | 2-NH₂ | 547 |
| 42 | 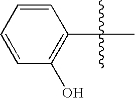 | 5-F | H | H | —CH₂— | 2-NH₂ | 529 |
| 43 | 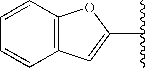 | 6-F | H | H | —CH₂— | 2-NH₂ | 553 |
| 44 | 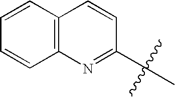 | 6-F | H | H | —CH₂— | 2-NH₂ | 564 |
| 45 | 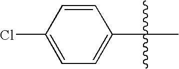 | H | H | H | —CH₂— | 2-NH₂ | 529 |
| 46 | 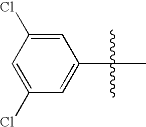 | 5-F | H | H | —CH₂— | 2-NH₂ | 581 |
| 47 | 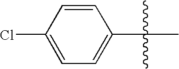 | 5-Cl | H | H | —CH₂— | 2-NH₂ | 563 |
| 48 | 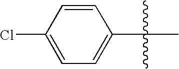 | 6-Cl | H | H | —CH₂— | 2-NH₂ | 563 |
| 49 | 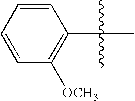 | 5-F | H | H | —CH₂— | 2-NH₂ | 543 |
| 50 | 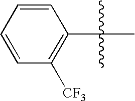 | 5-F | H | H | —CH₂— | 2-NH₂ | 581 |
| 51 | 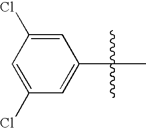 | 5-Cl | H | H | —CH₂— | 2-NH₂ | 597 |
| 52 | 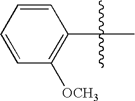 | 5-F | H | H | —CH₂— | 2-NH₂ | 597 |
| 53 | 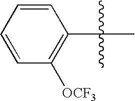 | 5-Br | H | H | —CH₂— | 2-NH₂ | 604 |
| 54 | 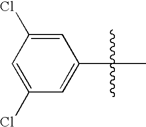 | 6-Cl | H | H | —CH₂— | 2-NH₂ | 597 |
| 55 | 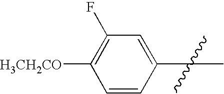 | 5-CH₃ | H | H | —CH₂— | 2-NH₂ | 571 |
| 56 | 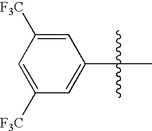 | 5-Cl | H | H | —CH₂— | 2-NH₂ | 665 |
| 57 | 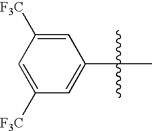 | 5-Br | H | H | —CH₂— | 2-NH₂ | 710 |
| 58 | 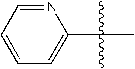 | 6-ethoxy | H | H | —CH₂— | 2-NH₂ | 540 |
| 59 | 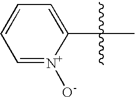 | 5-Cl | H | H | —CH₂— | 2-NH₂ | 546 |
| 60 | 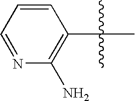 | H | H | H | —CH₂— | 2-NH₂ | 511 |
| 61 | 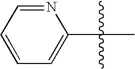 | 5-F | H | H | —CH₂— | H | 499 |
| 62 | 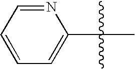 | 6-Cl | H | H | —CH₂— | 2-NH₂ | 530 |
| 63 | 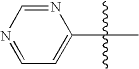 | 5-F | H | H | —CH₂— | 2-NH₂ | 515 |
| 64 | 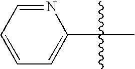 | 6-F | H | H | —CH₂— | 2-NH₂ | 514 |
| 65 | 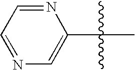 | 6-F | H | H | —CH₂— | 2-NH₂ | 515 |
| 66 | 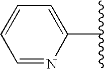 | 7-Cl | H | H | —CH₂— | 2-NH₂ | 531 |
| 67 | 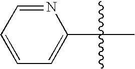 | H | H | H | —CH₂— | 2-NH₂ | 496 |
| 68 | 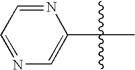 | 5-F | H | H | —CH₂— | 2-NH₂ | 515 |
| 69 | 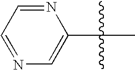 | 5-Cl | H | H | —CH₂— | 2-NH₂ | 531 |
| 70 | 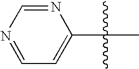 | 5-Cl | H | H | —CH₂— | 2-NH₂ | 531 |
| 71 | 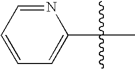 | 5,6-di-F | H | H | —CH₂— | 2-NH₂ | 532 |
| 72 | 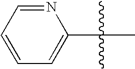 | 5-Br | H | H | —CH₂— | 2-NH₂ | 575 |
| 73 | 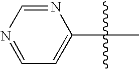 | 6-ethoxy | H | H | —CH₂— | 2-NH₂ | 541 |
| 74 | 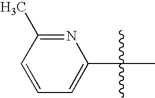 | 5-F | H | H | —CH₂— | 2-NH₂ | 528 |
| 75 | 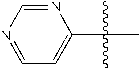 | 6-F | H | H | —CH₂— | 2-NH₂ | 515 |
| 76 | 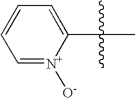 | 5-Br | H | H | —CH₂— | 2-NH₂ | 591 |
| 77 | 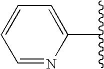 | 5-Cl | H | H | —CH₂— | 2-NH₂ | 530 |
| 78 | 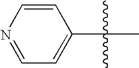 | 5-Cl | H | H | —CH₂— | 2-NH₂ | 530 |
| 79 | 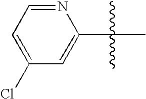 | 5-F | H | H | —CH₂— | 2-NH₂ | 548 |
| 80 | 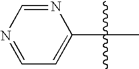 | 5-CF₃ | H | H | —CH₂— | 2-NH₂ | 565 |
| 81 | 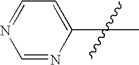 | H | H | H | —CH₂— | 2-NH₂ | 497 |
| 82 | 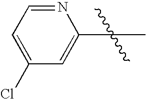 | 6,7-di-F | H | H | —CH₂— | 2-NH₂ | 567 |
| 83 | 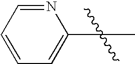 | 6,7-di-F | H | H | —CH₂— | 2-NH₂ | 532 |
| 84 | 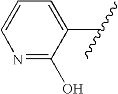 | 5-F | H | H | —CH₂— | 2-NH₂ | 530 |
| 85 | 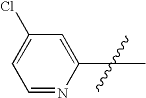 | 5-CF₃,7-F | H | H | —CH₂— | 2-NH₂ | 617 |
| 86 | 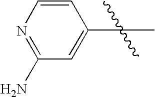 | 5-F | H | H | —CH₂— | 2-NH₂ | 529 |
| 87 | 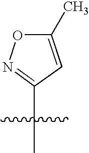 | H | H | H | —CH₂— | 2-NH₂ | 500 |
| 88 | 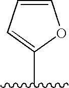 | H | H | H | —CH₂— | 2-NH₂ | 485 |
| 89 | 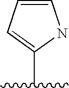 | H | H | H | —CH₂— | 2-NH₂ | 489 |
| 90 | 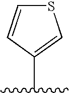 | 6-F | H | H | —CH₂— | 2-NH₂ | 514 |
| 91 | 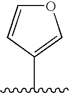 | 6-F | H | H | —CH₂— | 2-NH₂ | 503 |
| 92 | 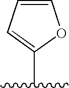 | 5-F | H | H | —CH₂— | 2-NH₂ | 503 |
| 93 | 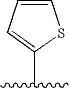 | H | H | H | —CH₂— | 2-NH₂ | 501 |
| 94 | 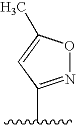 | 5-F | H | H | —CH₂— | 2-NH₂ | 518 |
| 95 | 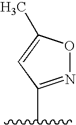 | 5-Cl | H | H | —CH₂— | 2-NH₂ | 534 |
| 96 | 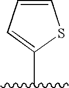 | 5-F | H | H | —CH₂— | 2-NH₂ | 519 |
| 97 | 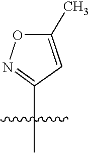 | 6,7-di-F | H | H | —CH₂— | 2-NH₂ | 536 |
| 98 | 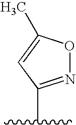 | 5-Br | H | H | —CH₂— | 2-NH₂ | 579 |
| 99 | 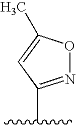 | 6-ethoxy | H | H | —CH₂— | 2-NH₂ | 544 |
| 100 | 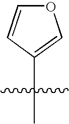 | 5-F | H | H | —CH₂— | 2-NH₂ | 503 |
| 101 | 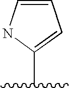 | 5-Br | H | H | —CH₂— | 2-NH₂ | 563 |
| 102 | 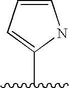 | 5-F | H | H | —CH₂— | 2-NH₂ | 502 |
| 103 | 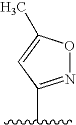 | 5-CF₃ | H | H | —CH₂— | 2-NH₂ | 568 |
| 104 | 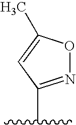 | 5-CF₃,7-F | H | H | —CH₂— | 2-NH₂ | 586 |
| 105 | 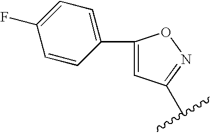 | 5-F | H | H | —CH₂— | 2-NH₂ | 598 |
| 106 | 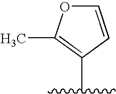 | 5-F | H | H | —CH₂— | 2-NH₂ | 517 |
| 107 | 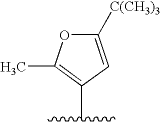 | 5-F | H | H | —CH₂— | 2-NH₂ | 573 |
| 108 | 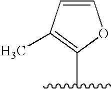 | 5-F | H | H | —CH₂— | 2-NH₂ | 517 |
| 109 | CH₃—S— | 5-F | H | H | —CH₂— | 2-NH₂ | 483 |
| 110 | CH₃—CH₂—S— | 5-F | H | H | —CH₂— | 2-NH₂ | 497 |
| 111 | CH₃—SO₂— | 5-F | H | H | —CH₂— | 2-NH₂ | 515 |
| 112 | 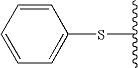 | 5-F | H | H | —CH₂— | 2-NH₂ | 546 |
| 113 | 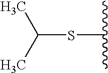 | 5-F | H | H | —CH₂— | 2-NH₂ | 511 |
| 114 | 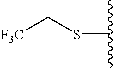 | 5-F | H | H | —CH₂— | 2-NH₂ | 551 |
| 115 | 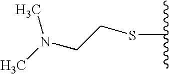 | 5-F | H | H | —CH₂— | 2-NH₂ | 541 |
| 116 | HS— | 5-F | H | H | —CH₂— | 2-NH₂ | 469 |
| 117 | CH₃—S— | 5-F | H | 2-CH₃ | —CH₂— | 2-NH₂ | 497 |
| 118 | CH₃—S— | 5-F | F | H | —CH₂— | 2-NH₂ | 501 |
| 119 | 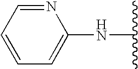 | 5-F | H | H | —CH₂— | 2-NH₂ | 529 |
| 120 | 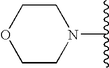 | 5-F | H | H | —CH₂— | 2-NH₂ | 522 |
| 121 | 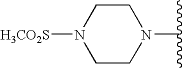 | 5-F | H | H | —CH₂— | 2-NH₂ | 600 |
| 123 | 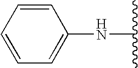 | 5-F | H | H | —CH₂— | 2-NH₂ | 528 |
| 124 | 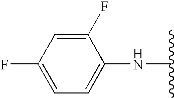 | 5-F | H | H | —CH₂— | 2-NH₂ | 564 |
| 125 | 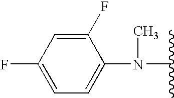 | 5-F | H | H | —CH₂— | 2-NH₂ | 578 |
| 126 | 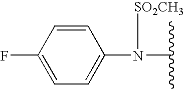 | 5-F | H | H | —CH₂— | 2-NH₂ | 625 |
| 127 | 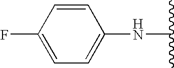 | 5-F | H | H | —CH₂— | 2-NH₂ | 546 |
| 128 | 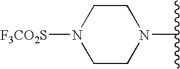 | 5-F | H | H | —CH₂— | 2-NH₂ | 654 |
| 129 | CH₃—O—(CH₂)₂—NH— | 5-F | H | H | —CH₂— | 2-NH₂ | 510 |
| 130 | 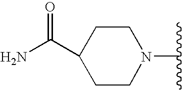 | 5-F | H | H | —CH₂— | 2-NH₂ | 564 |
| 131 | 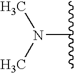 | 5-F | H | H | —CH₂— | 2-NH₂ | 480 |
| 132 | CH₃—O— | 5-F | H | H | —CH₂— | 2-NH₂ | 467 |
| 133 | CH₃—CH₂—O— | 5-F | H | H | —CH₂— | 2-NH₂ | 481 |
| 134 | CH₃—O—(CH₂)₂—O— | 5-F | H | H | —CH₂— | 2-NH₂ | 511 |
| 135 | (CH₃)₂—CH—O— | 5-F | H | H | —CH₂— | 2-NH₂ | 495 |
| 136 | 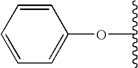 | 5-F | H | H | —CH₂— | 2-NH₂ | 529 |
| 137 | 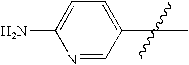 | H | H | H | —CH₂— | 2-NH₂ | 511 |
| 138 | 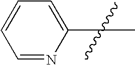 | 5-CF₃,7-F | H | H | —CH₂— | 2-NH₂ | 582 |
| 139 | 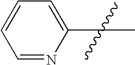 | 5-F | H | H | 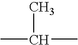 | 2-NH₂ | 528 |
| 140 | 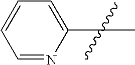 | 5-F | F | H | —CH₂— | 2-NH₂ | 532 |
| 141 | 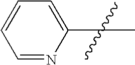 | 5-F | OH | H | —CH₂— | 2-NH₂ | 530 |
| 142 | 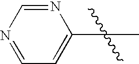 | 5-F | H | H | 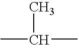 | 2-NH₂ | 529 |
| 143 | 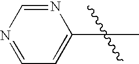 | 5-F | H | H | 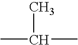 | 2-NH₂ | 529 |
| 144 | 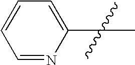 | 5-F | —CH₃ | H | —CH₂— | 2-NH₂ | 528 |
| 145 | 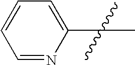 | 6-F | H | H | 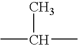 | 2-NH₂ | 528 |
| 146 | H | 5-F | H | H | —CH₂— | 2-NH₂ | 437 |
| 147 | 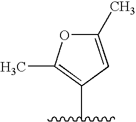 | 5-F | H | H | —CH₂— | 2-NH₂ | 531 |
| 148 | 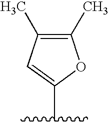 | 5-F | H | H | —CH₂— | 2-NH₂ | 531 |
| 149 | 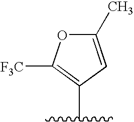 | 5-F | H | H | —CH₂— | 2-NH₂ | 585 |
| 150 | 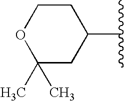 | 5-F | H | H | —CH₂— | 2-NH₂ | 549 |
| 151 | 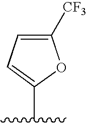 | 5-F | H | H | —CH₂— | 2-NH₂ | 571 |
| 152 | 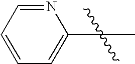 | H | F | H | —CH₂— | 2-NH₂ | 514 |
| 153 | (CH₃)₂N—(CH₂)₂—NH— | 5-F | H | H | —CH₂— | 2-NH₂ | 523 |
| 154 | CH₃—S— | 5-F | H | H | 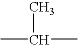 | 2-NH₂ | |
| 155 | 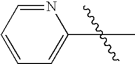 | 5-F | H | 2-CH₃ | —CH₂— | 2-NH₂ | |
| 156 | 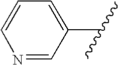 | 5-F | H | H | —CH₂— | 2-NH₂ | 514 |
| 157 | 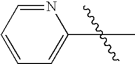 | 5-F | H | H | —CH₂— | 3-NH₂ | 514 |
| 158 | 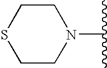 | 5-F | H | H | —CH₂— | 2-NH₂ | 539 |
| 159 | 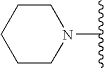 | 5-F | H | H | —CH₂— | 2-NH₂ | 520 |
| 160 | CH₃CH₂O— | 5-F | F | H | —CH₂— | 2-NH₂ | |
| 161 | 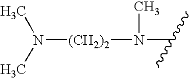 | 5-F | H | H | —CH₂— | 2-NH₂ | 538 |
| 162 | 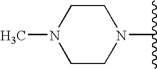 | 5-F | H | H | —CH₂— | 2-NH₂ | 535 |
| 163 | 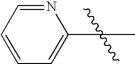 | 5-F | H | 5-OH | —CH₂— | 2-NH₂ | 530 |
| 164 | 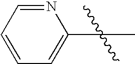 | 5-F | F | H | —CH₂— | 3-NH₂ | 532 |
| 165 | 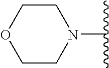 | 5-F | F | H | —CH₂— | 2-NH₂ | 540 |
| 166 | 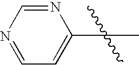 | 5-F | H | H | —CH₂— | 3-NH₂ | 515 |

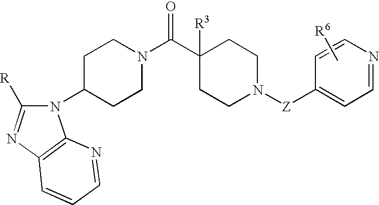

| No. | R | R³ | Z | R⁶ | MS (MH⁺) |
|---|---|---|---|---|---|
| 167 | 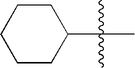 | H | —CH₂— | 2-NH₂ | 502 |
| 168 | —CH₂OCH₃ | H | —CH₂— | 2-NH₂ | 464 |
| 169 | 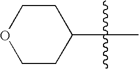 | H | —CH₂— | 2-NH₂ | 504 |
| 170 | 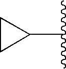 | H | —CH₂— | 2-NH₂ | 460 |
| 171 | (CH₃)₂—CH— | H | —CH₂— | 2-NH₂ | 462 |
| 172 | 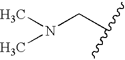 | H | —CH₂— | 2-NH₂ | 477 |
| 173 | 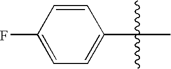 | H | —CH₂— | 2-NH₂ | 514 |
| 174 | 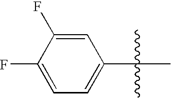 | H | —CH₂— | 2-NH₂ | 532 |
| 175 | 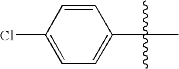 | H | —CH₂— | 2-NH₂ | 530 |
| 176 | 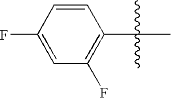 | H | —CH₂— | 2-NH₂ | 532 |
| 177 | 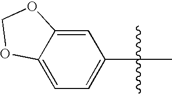 | H | —CH₂— | 2-NH₂ | 540 |
| 178 | 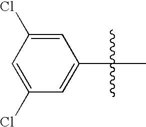 | H | —CH₂— | 2-NH₂ | 564 |
| 179 | 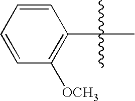 | H | —CH₂— | 2-NH₂ | 526 |
| 180 | 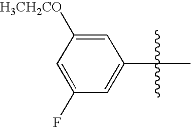 | H | —CH₂— | 2-NH₂ | 558 |
| 181 | 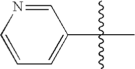 | H | —CH₂— | 2-NH₂ | 497 |
| 182 | 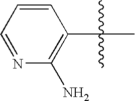 | H | —CH₂— | 2-NH₂ | 512 |
| 183 | 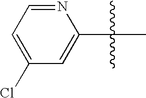 | H | —CH₂— | 2-NH₂ | 531 |
| 184 | 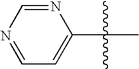 | H | —CH₂— | 2-NH₂ | 498 |
| 185 | 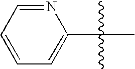 | H | —CH₂— | 2-NH₂ | 497 |
| 186 | 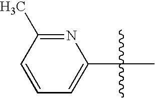 | H | —CH₂— | 2-NH₂ | 511 |
| 187 | 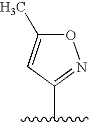 | H | —CH₂— | 3-NH₂ | 501 |
| 188 | 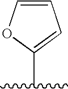 | H | —CH₂— | 2-NH₂ | 486 |
| 189 | 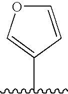 | H | —CH₂— | 2-NH₂ | 486 |
| 190 | 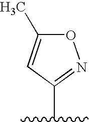 | H | —CH₂— | 2-NH₂ | 501 |
| 191 | 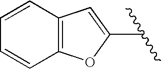 | H | —CH₂— | 2-NH₂ | 536 |
| 192 | 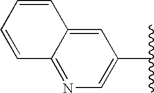 | H | —CH₂— | 2-NH₂ | 547 |
| 193 | 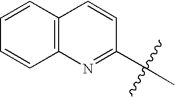 | H | —CH₂— | 2-NH₂ | 547 |
| 194 | 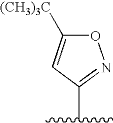 | H | —CH₂— | 2-NH₂ | 543 |
| 195 | 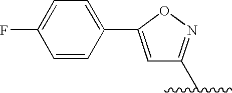 | H | —CH₂— | 2-NH₂ | 581 |
| 196 | 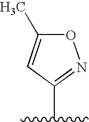 | F | —CH₂— | 2-NH₂ | 519 |
| 197 | 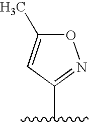 | F | 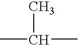 | 2-NH₂ | 515 |
| 198 | 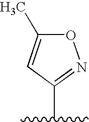 | OH | —CH₂— | 2-NH₂ | 517 |
| 199 | 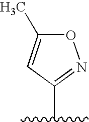 | 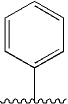 | —CH₂— | 2-NH₂ | 577 |
| 200 | 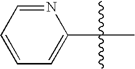 | F | —CH₂— | 2-NH₂ | 515 |
| 201 | 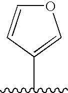 | F | —CH₂— | 2-NH₂ | 504 |
| 202 | 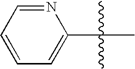 | H | —CH₂— | 3-NH₂ | 497 |
| 203 | 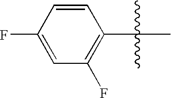 | H | —CH₂— | 3-NH₂ | 532 |
| 304 | 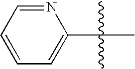 | F | —CH₂— | 3-NH₂ | 515 |
| 205 | 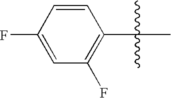 | F | —CH₂— | 3-NH₂ | 550 |

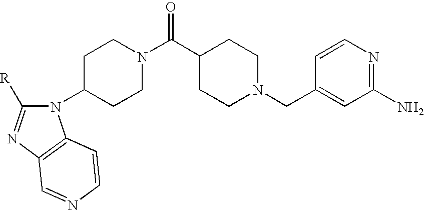

| | | Physical |
|---|---|---|
| | | Data |
| No. | R | MS (MH⁺) |
| 206 | —CH₃ | 434 |
| 207 | 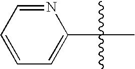 | 497 |
| 208 | 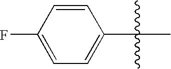 | 514 |
| 209 | 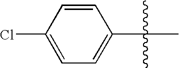 | 530 |

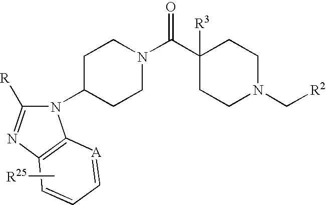

| | | | | | | Physical |
|---|---|---|---|---|---|---|
| | | | | | | Data |
| No. | R | R²⁵ | A | R³ | R² | MS (MH⁺) |
| 210 | 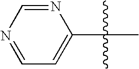 | 5-Cl | C | H | 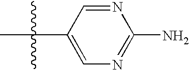 | 532 |
| 211 | 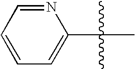 | 5-F | C | H | 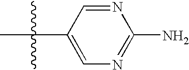 | 515 |
| 212 | 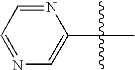 | 5-Cl | C | H | 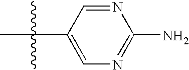 | 532 |
| 213 | 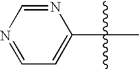 | 5-F | C | H | 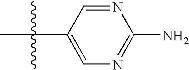 | 516 |
| 214 | 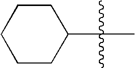 | H | N | H | 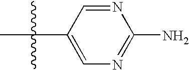 | 503 |
| 215 | 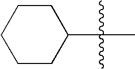 | H | N | H | 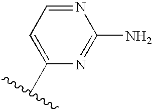 | 503 |
| 216 | (CH₃)₂CH— | H | N | H | 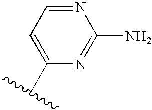 | 463 |
| 217 | 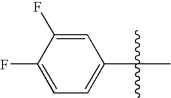 | 5-F | C | H | 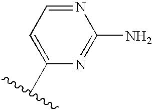 | 550 |
| 218 | 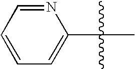 | 5-F | C | H | 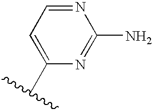 | 515 |
| 219 | 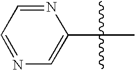 | 5-Cl | C | H | 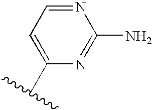 | 532 |
| 220 | 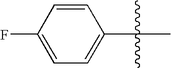 | 6-Cl | C | H | 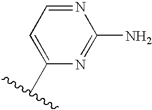 | 548 |
| 221 | 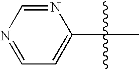 | 5-F | C | H | 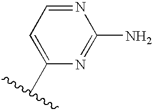 | 516 |
| 222 | 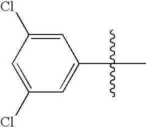 | 6-Cl | C | H | 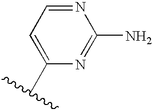 | 600 |
| 223 | 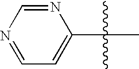 | 5-Cl | C | H | 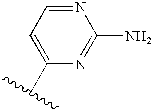 | 532 |
| 224 | 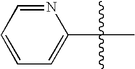 | 6-F | C | H | 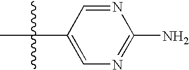 | 515 |
| 225 | 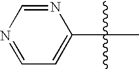 | H | N | H | 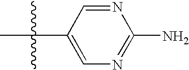 | 499 |
| 226 | 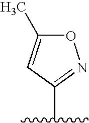 | H | N | H | 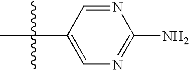 | 502 |
| 227 | 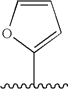 | H | N | H | 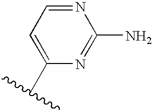 | 487 |
| 228 | 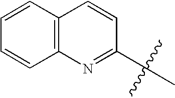 | H | N | H | 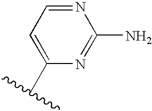 | 548 |
| 229 | 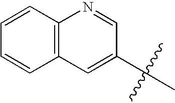 | H | N | H | 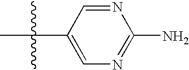 | 548 |
| 230 | 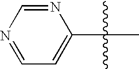 | H | N | H | 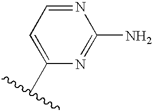 | 499 |
| 231 | 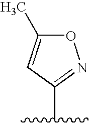 | H | N | H | 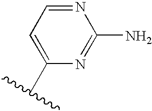 | |
| 232 | 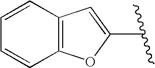 | H | N | H | 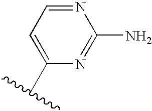 | 537 |
| 233 | 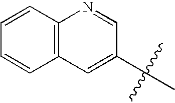 | H | N | H | 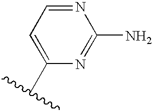 | 548 |
| 234 | 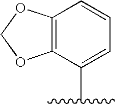 | H | N | H | 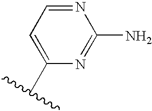 | 541 |
| 235 | 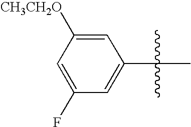 | H | N | H | 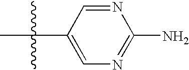 | 559 |
| 236 | 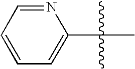 | H | N | H | 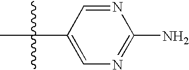 | 498 |
| 237 | 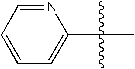 | 5-F | C | F | 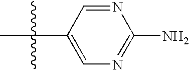 | 533 |
| 238 | 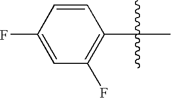 | 5-F | C | H | 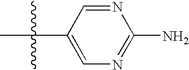 | 550 |
| 239 | 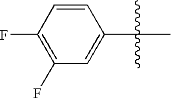 | 5-F | C | H | 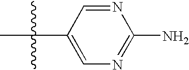 | 550 |
| 240 | 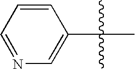 | 5-F | C | H | 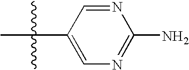 | 515 |
| 241 | 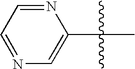 | 5-F | C | H | 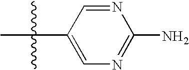 | 516 |
| 242 | 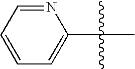 | H | C | H | 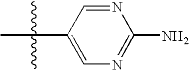 | 497 |
| 243 | (CH₃)₂N—CH₂— | H | N | H | 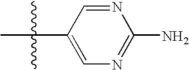 | |
| 244 | 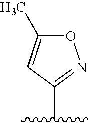 | 5-F | C | H | 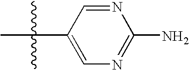 | 519 |
| 245 | 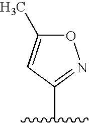 | H | C | H | 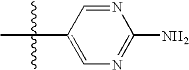 | 501 |
| 246 | 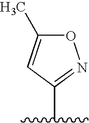 | 5,6-di-F | C | H | 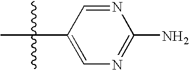 | 537 |
| 247 | 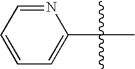 | 5-F | C | H | 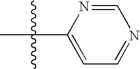 | 500 |
| 248 | 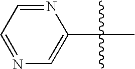 | 5,6-di-F | C | H | 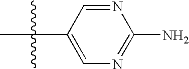 | 534 |
| 249 | 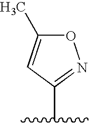 | 5-F | C | F | 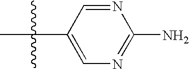 | 537 |
| 250 | 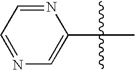 | 5-F | C | F | 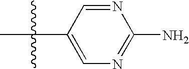 | 534 |
| 251 | 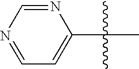 | 5-F | C | F | 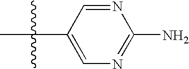 | 534 |
| 252 | 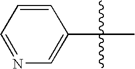 | 5-F | C | F | 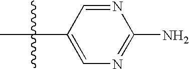 | 533 |
| 253 | 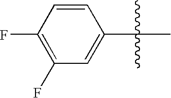 | 5-F | C | F | 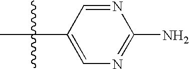 | 568 |
| 254 | 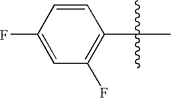 | 5-F | C | F | 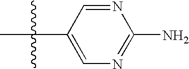 | 568 |
| 255 | 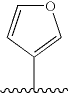 | H | N | H | 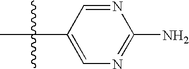 | 487 |
| 256 | 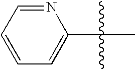 | H | C | F | 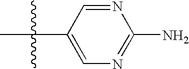 | 515 |
| 257 | 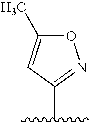 | H | C | F | 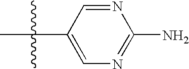 | 519 |
| 258 | 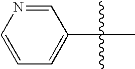 | H | N | F | 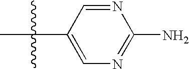 | 516 |
| 259 | 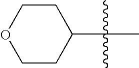 | H | N | H | 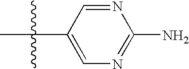 | 505 |
| 260 | 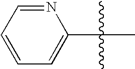 | H | N | F | 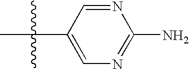 | 516 |
| 261 | 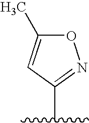 | H | N | F | 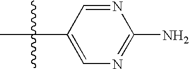 | 520 |
| 262 | 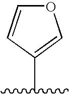 | 5-F | C | H | 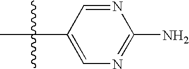 | 504 |
| 263 | 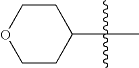 | 5-F | C | H | 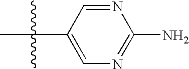 | 522 |
| 264 | 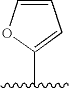 | 5-F | C | H | 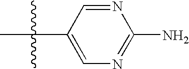 | 504 |
| 265 | 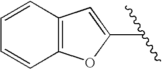 | H | N | H | 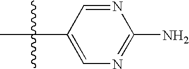 | 537 |
| 266 | (CH₃)₂N—CH₂— | H | N | F | 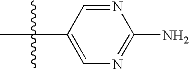 | 496 |
| 267 | 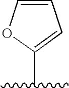 | H | N | F | 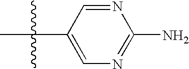 | 505 |
| 268 | CH₃CH₂—O— | 5-F | C | H | 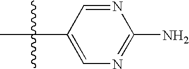 | |
| 269 | CH₃—S— | 5-F | C | H | 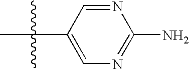 | |
| 270 | CH₃CH₂—O— | 5-F | C | F | 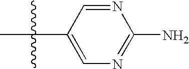 | 500 |
| 271 | 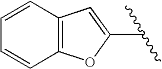 | H | N | F | 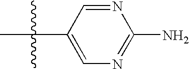 | 555 |
| 272 | 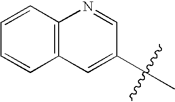 | H | N | F | 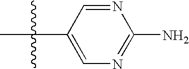 | 566 |
| 273 | 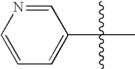 | H | N | H | 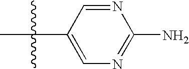 | 498 |
| 274 | 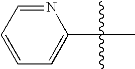 | 5,6- di-F | C | F | 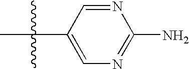 | 551 |
| 275 | 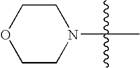 | 5-F | C | F | 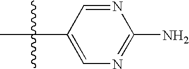 | 541 |
| 276 | 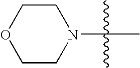 | 5-F | C | H | 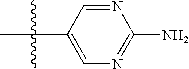 | 523 |
| 277 | 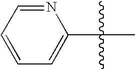 | 5-F | C | H | 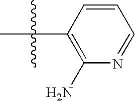 | |
| 278 | 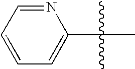 | 5-F | C | H | 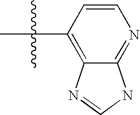 | 539 |
| 279 | 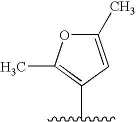 | H | N | H | 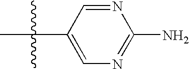 | 515 |
| 280 | 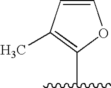 | H | N | H | 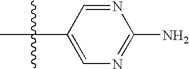 | 501 |
| 281 | 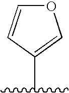 | H | N | F | 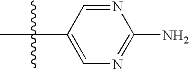 | 505 |
| 282 | 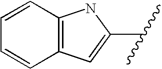 | H | N | H | 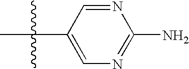 | 536 |
| 283 | 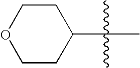 | H | N | F | 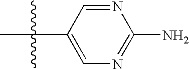 | 523 |
| 284 | 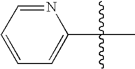 | 5-F | C | F | 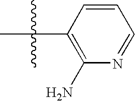 | |
| 285 | 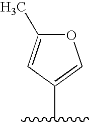 | H | N | H | 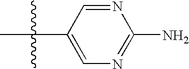 | 501 |
| 286 | 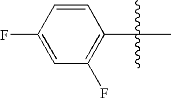 | H | N | H | 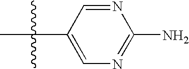 | 533 |
| 287 | 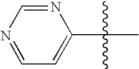 | H | N | F | 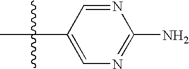 | 517 |
| 288 | 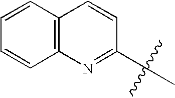 | H | N | H | 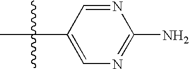 | 548 |
| 289 | 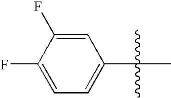 | H | N | H | 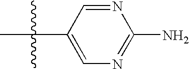 | 533 |
| 290 | 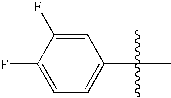 | H | C | F | 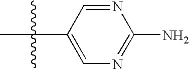 | |
| 291 | 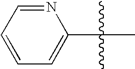 | H | N | F | 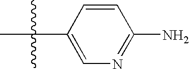 | 515 |
| 292 | 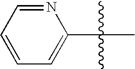 | 5-F | C | F | 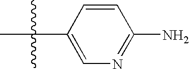 | 532 |
| 293 | 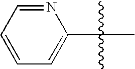 | 5-F | C | H | 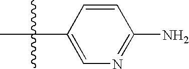 | 514 |
| 294 | 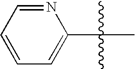 | H | N | H | 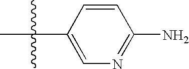 | 497 |
| 295 | (CH₃)₂N— | 5-F | C | F | 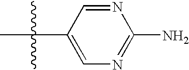 | |
| 296 | CH₃CH₂—S— | 5-F | C | F | 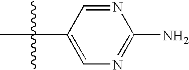 | |
| 297 | CH₃—O— | 5-F | C | F | 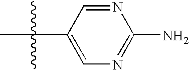 | |
| 298 | 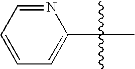 | H | N | H | 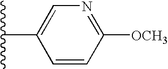 | 512 |
| 299 | 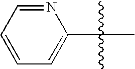 | H | N | F | 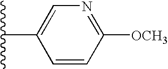 | 530 |
| 300 | 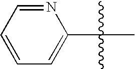 | 5-F | C | F | 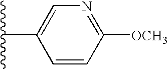 | 547 |
| 301 | 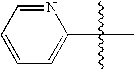 | 5-F | C | H | 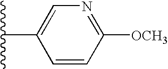 | 529 |
| 302 | 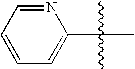 | 5-F | C | H | 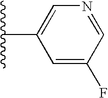 | 517 |
| 303 | 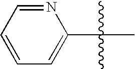 | 5-F | C | F | 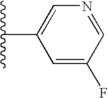 | 535 |
| 304 | 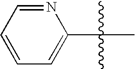 | H | N | H | 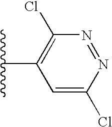 | 551 |
| 305 | 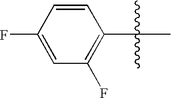 | H | N | F | 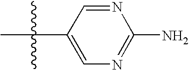 | 551 |
| 306 | 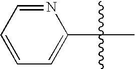 | 5-F | C | H | 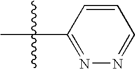 | 500 |
| 307 | 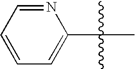 | 5-F | C | H | 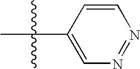 | 500 |
| 308 | 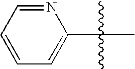 | 5-F | C | F | 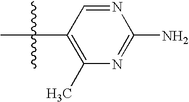 | 547 |
| 309 | (CH₃CH₂)₂N— | 5-F | C | F | 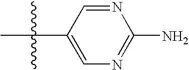 | |
| 310 | 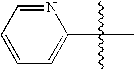 | H | N | H | 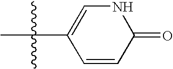 | 498 |
| 311 | 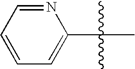 | H | N | F | 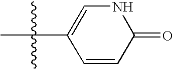 | 516 |
| 312 | 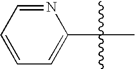 | 5-F | C | H | 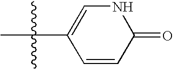 | 515 |
| 313 | 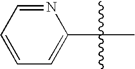 | 5-F | C | F | 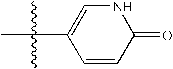 | 533 |
| 314 | 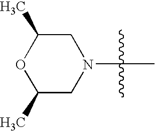 | 5-F | C | F | 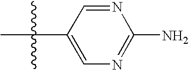 | |
| 315 | CH₃—S— | H | N | F | 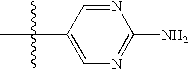 | |
| 316 | CH₃CH₂—O— | H | N | F | 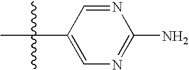 | |
| 317 | 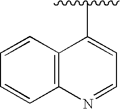 | H | N | F | 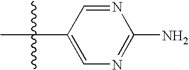 | 566 |
| 318 | 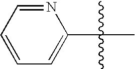 | H | N | F | 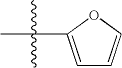 | 489 |
| 319 | 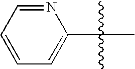 | H | N | F | 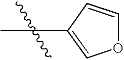 | 489 |
| 320 | 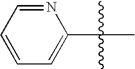 | H | N | F | 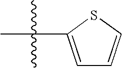 | 505 |
| 321 | 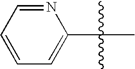 | H | N | F | 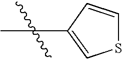 | 505 |
| 322 | 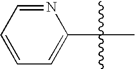 | 5-F | C | F | 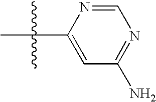 | 533 |
| 323 | 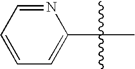 | H | N | F | 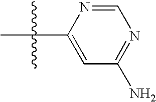 | 516 |
| 325 | 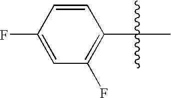 | H | N | F | 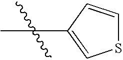 | 540 |
| 325 | 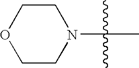 | H | N | F | 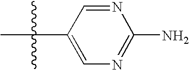 | |
| 326 | (CH₃)₂CH—O— | 5-F | C | F | 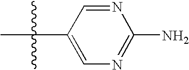 | |
| 327 | 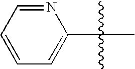 | H | N | F | 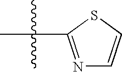 | 506 |
| 328 | 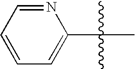 | H | N | F | 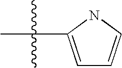 | 488 |
| 329 | 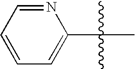 | H | N | F | 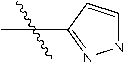 | 489 |
| 330 | 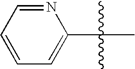 | H | N | F | 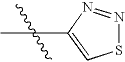 | 507 |
| 331 | 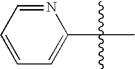 | H | N | F | 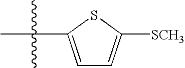 | 551 |
| 332 | 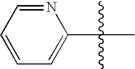 | H | N | F | 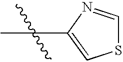 | 506 |
| 333 | 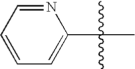 | H | N | F | 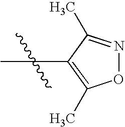 | 518 |
| 334 | 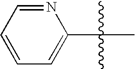 | H | N | F | 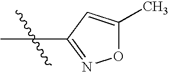 | 504 |
| 335 | CH₃—O— | H | N | F | 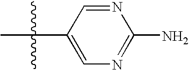 | |
| 336 | 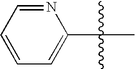 | H | N | F | 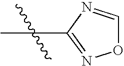 | 491 |
| 337 | 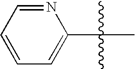 | H | N | F | 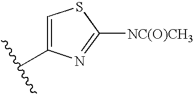 | 563 |
| 338 | 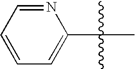 | 5-F | C | H | 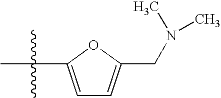 | 545 |
| 339 | 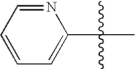 | 5-F | C | H | 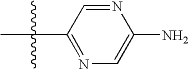 | 533 |
| 340 | 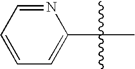 | H | N | F | 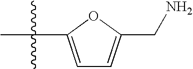 | 518 |
| 341 | 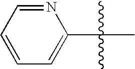 | 5-F | C | H | 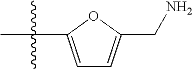 | 535 |
| 342 | 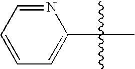 | H | N | F | 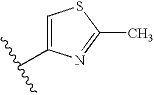 | 520 |
| 343 | 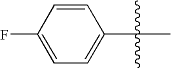 | 6-Cl | C | H | 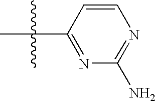 | 548 |
| 345 | 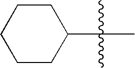 | H | N | H | 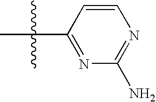 | |
| 346 | (CH₃)₂—CH— | H | N | H | 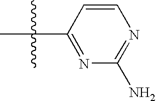 | |

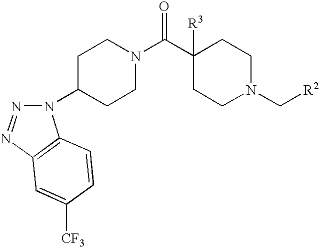

| | | | Physical |
|---|---|---|---|
| | | | Data |
| No. | R³ | R² | MS (MH⁺) |
| 347 | H | 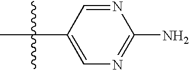 | 489 |
| 348 | F | 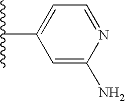 | 506 |
| 349 | F | 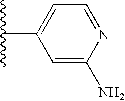 | 488 |
| 350 | F | 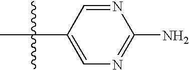 | 507 |
| 351 | F | 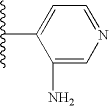 | 506 |

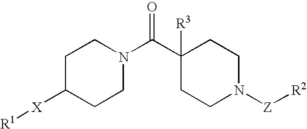

| | | | | | Physical |
|---|---|---|---|---|---|
| | | | | | Data |
| No. | R¹—X— | Z | R³ | R² | MS (MH⁺) |
| 352 | 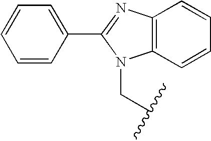 | —CH₂— | H | 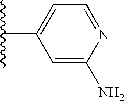 | 509 |
| 353 | 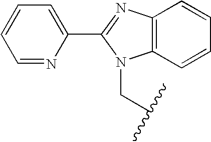 | —CH₂— | H | 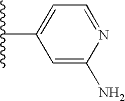 | 510 |
| 354 | 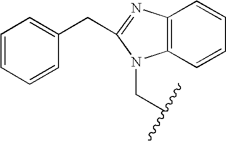 | —CH₂— | H | 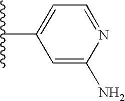 | 524 |
| 355 | 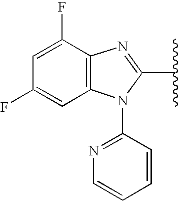 | —CH₂— | H | 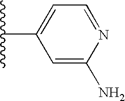 | 532 |
| 356 | 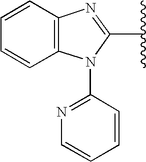 | —CH₂— | H | 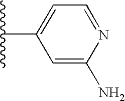 | 496 |
| 357 | 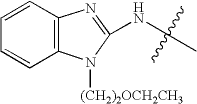 | —CH₂— | H | 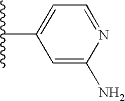 | 506 |
| 358 | 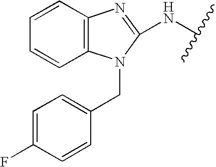 | —CH₂— | H | 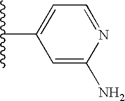 | 542 |
| 359 | 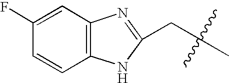 | —CH₂— | H | 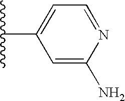 | 451 |
| 360 | 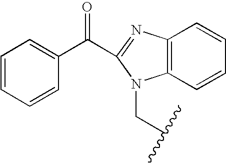 | —CH₂— | H | 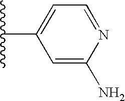 | 537 |
| 361 | 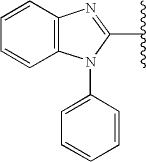 | —CH₂— | H | 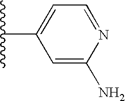 | 495 |
| 362 | 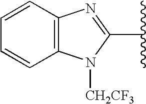 | —CH₂— | H | 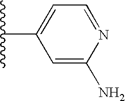 | 501 |
| 363 | 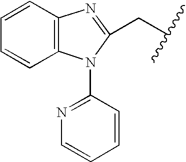 | —CH₂— | H | 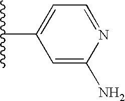 | 510 |
| 364 | 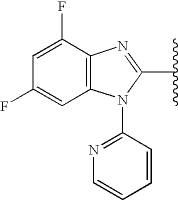 | —CH₂— | H | 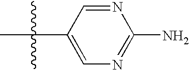 | 533 |
| 365 | 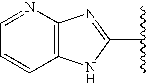 | —CH₂— | H | 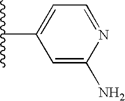 | 420 |
| 366 | 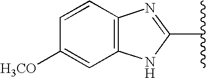 | —CH₂— | H | 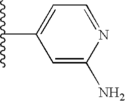 | 449 |
| 367 | 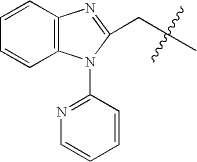 | —CH₂— | H | 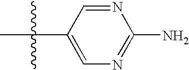 | 497 |
| 368 | 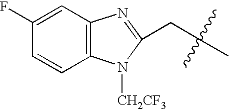 | —CH₂— | H | 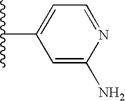 | 533 |
| 369 | 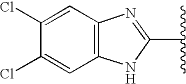 | —CH₂— | H | 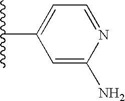 | 487 |
| 370 | 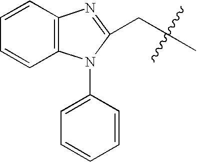 | —CH₂— | H | 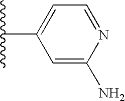 | 509 |
| 371 | 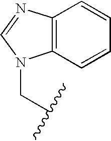 | —CH₂— | H | 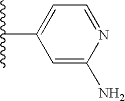 | |
| 372 | 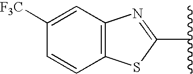 | —CH₂— | H | 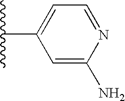 | |
| 373 | 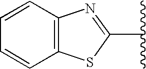 | —CH₂— | H | 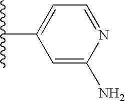 | |
| 374 | 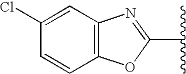 | —CH₂— | H | 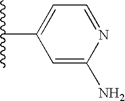 | |
| 375 | 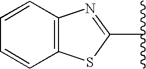 | —(CH₂)₃— | H | 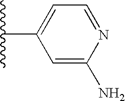 | |
| 376 | 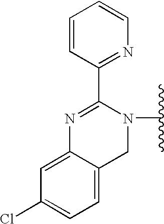 | —CH₂— | H | 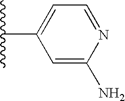 | |
| 377 | 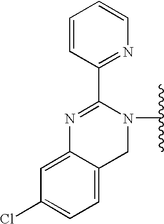 | —CH₂— | F | 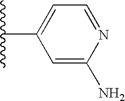 | |

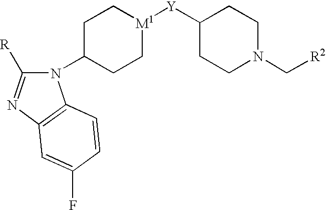

| | | | | | Physical |
|---|---|---|---|---|---|
| | | | | | Data |
| No. | R | M¹ | Y | R² | MS (MH⁺) |
| 378 | 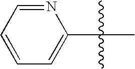 | CH | —CH₂— | 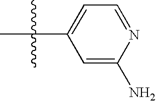 | 500 |
| 379 | 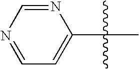 | N | —NH— | 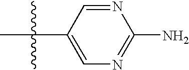 | 502 |
| 380 | 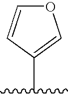 | N | —NH— | 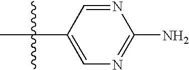 | 490 |
| 381 | 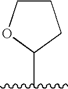 | N | —NH— | 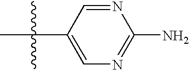 | 494 |
| 382 | 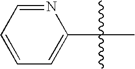 | N | —NH— | 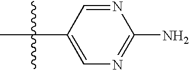 | 501 |
| 383 | 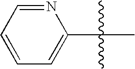 | N | —NH— | 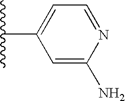 | 500 |

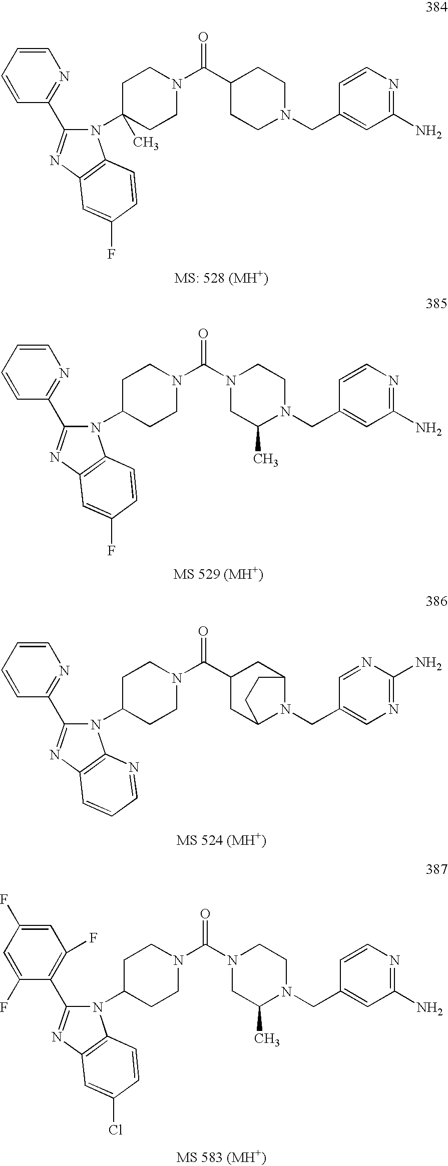

### Example 18

### Preparation of Compound 388

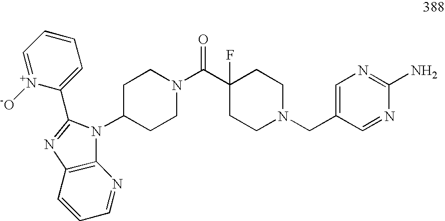

### Step 1—Synthesis of Compound 388A

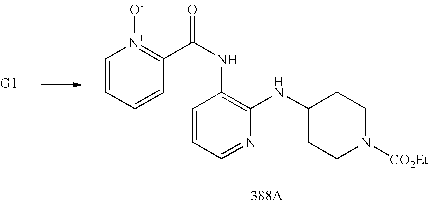

To a solution of compound G1 (see Example 7, Step 1; 2.3 g, 8.9 mmol) in CH₂Cl₂— DMF (1:1, 50 mL) was sequentially added picolinic acid N-oxide (1.5 g, 10.6 mmol), EDCI (2.6 g, 13.3 mmol) and HOBT (1.8 g, 13.3 mmol). The reaction was stirred at 70° C. for about 15 hours. The reaction mixture was concentrated in vacuo and the residue obtained was diluted with EtOAc, washed three times with 5% aqueous NaOH, dried over Na₂SO₄, and concentrated in vacuo. The resulting residue was purified using flash chromatography (50% EtOAc/hexane) to provide compound 388A (2.5 g, 74%).

### Step 2—Synthesis of Compound 388B

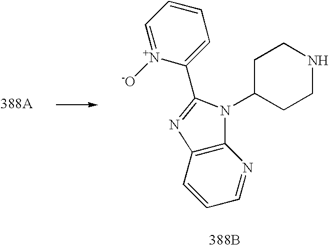

Using the method described in Example 5, Step 4, compound 388A was converted to compound 388B.

### Step 3—Synthesis of Compound 388C

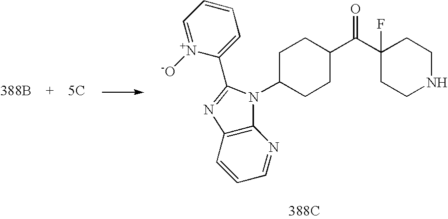

To a solution of compound 388B (0.66 g, 2.2 mmol) in DMF (15 mL) was sequentially added compound 5C (see Example 16, Step 2; 0.62 g, 2.5 mmol), 1-propanephosphonic acid cyclic anhydride (3.3 ml, 11.2 mmol, 50 wt. % in EtOAc) and N-ethylmorpholine (1.4 ml, 10.7 mmol). The mixture was stirred at 50° C. for 3 hours. The reaction mixture was concentrated in vacuo and diluted with EtOAc. The solution was washed three times with 5% aqueous NaOH, dried over Na₂SO₄, concentrated in vacuo and subjected to flash chromatography (10% 2N NH₃—CH₃OH/EtOAc). The material was then taken up in CH₂Cl₂ (20 mL) and treated with 4 M HCl-dioxane (4 mL). After stirring for about 15 hours at 20° C., the reaction was carefully basified with 10% aqueous NaOH and extracted with CH₂Cl₂. The combined organic layers were dried over Na₂SO₄, concentrated in vacuo and the residue obtained was purified using flash chromatography (30% 2N NH₃—CH₃OH/EtOAc) to provide compound 388C as a white solid (0.08g, 10%).

### Step 4—Synthesis of Compound 388

Using the method described in Example 5, Step 5, compound 388C was converted to compound 388.

### Example 19

### Preparation of Compound 389

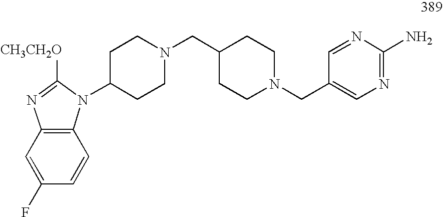

### Step 1—Synthesis of Compound 389C

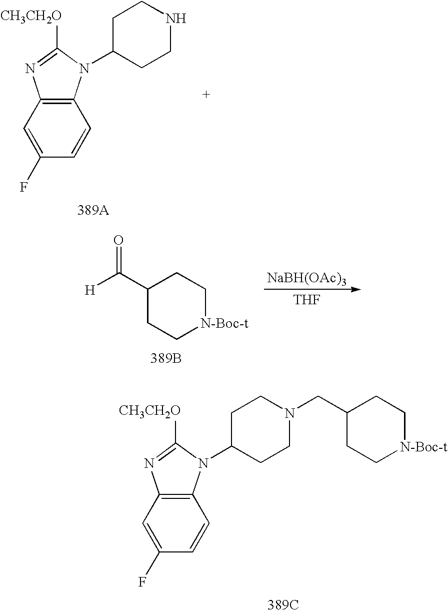

To a solution of 389A (300 mg, 1.14 mmol) in THF (15 mL) was added a solution of 389B (292 mg, 1.37 mmol) in THF (1 mL), followed by NaBH(OAc)₃ (483 mg, 2.28 mmol). The reaction was allowed to stir at room temperature for 39 h, then additional NaBH(OAc)₃ (242 mg, 1.14 mmol) was added and the reaction was allowed to stir at room temperature continued for an additional 3 days. The reaction mixture was then filtered and the collected solids washed thoroughly with CH₂Cl₂. The combined filtrate and washings were concentrated in vacuo, and the resulting residue was partitioned between EtOAc (60 mL) and a solution of water (2.5 mL), 2M Na₂CO₃ (6.5 mL) and 6N NaOH (5 mL). The aqueous layer was further extracted with EtOAc (3×15 mL) and the combined organic extracts were washed with brine (5 mL), then dried over anhydrous MgSO₄. Drying agent was removed by filtration, and the filtrate was concentrated in vacuo and the resulting residue was purified using silica gel flash chromatography (EtOAc/hexanes=1:1) to provide compound 389C as a mixture of colorless gum and white foam, which solidified upon standing (368 mg, 70%). ES-MS: 461 (MH⁺; 100%).

### Step 2—Synthesis of Compound 389D

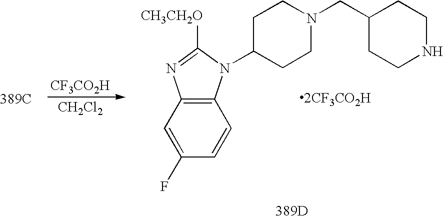

To a stirred, ice-cold solution of compound 389C (358 mg, 0.777 mL) in CH₂Cl₂ (7 mL) was added via syringe cold, neat TFA (576 microliters, 886 mg, 7.77 mmol). The resulting reaction was stirred in an ice-water bath for 30 min, then stirred at room temperature for 29.5 hours. Volatiles were removed in vacuo, and the gummy residue obtained was triturated (magnetic stirrer) with Et₂O (35 mL) for 16 hours. Filtration and washing of the collected solid product with Et₂O provided the bis-trifluoroacetate salt of compound 389D as a white powder (449 mg, 98%).

### Step 3—Synthesis of Compound 389

To a stirred suspension of compound 389D (100 mg, 0.170 mmol) in CH₂Cl₂ (5 mL) was added Et₃N (47.4 microliters, 34.4 mg, 0.340 mmol). To the resulting solution was added compound 5G (25.1 mg, 0.204 mmol), followed by NaBH(OAc)₃ (72.1 mg, 0.340 mmol). After stirring at room temperature for 66 h, TLC revealed the presence of unchanged starting materials in the light yellow reaction suspension. Therefore, another quantity of NaBH(OAc)₃ (72.1 mg, 0.340 mmol) was added and stirring at room temperature continued for a total of 90 hours. The reaction mixture was then filtered and collected solids washed thoroughly with CH₂Cl₂. The combined filtrate and washings were stripped of solvent under vacuum, and the residue was partitioned between EtOAc (20 mL) and a solution consisting of water (0.6 mL), 2M Na₂CO₃ (1.5 mL) and 6N NaOH (1.2 mL). The aqueous layer was further extracted with EtOAc (3×5 mL). The combined extracts were washed with brine (2 mL) and dried over anhydrous MgSO₄. Drying agent was removed by filtration, and the filtrate was concentrated in vacuo. The residue was purified by preparative TLC (silica gel; CH₂Cl₂/CH₃OH/conc. NH₄OH=90:9:1) to obtain the title compound as a light beige foam (36 mg, 45%). FABMS: 468 (MH⁺; 100%).

Using the methods described above in Examples 1-8, the following compounds were prepared:

| | | Mass Spec |
|---|---|---|
| No. | Structure | (M + H) |
| 390 | 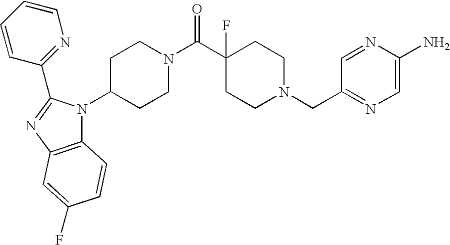 | 533 (ESMS) |
| 391 | 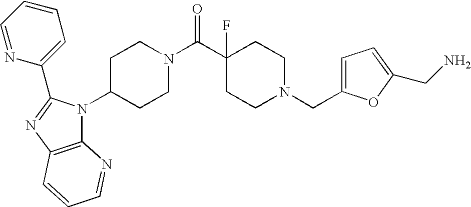 | 518 (ESMS) |
| 392 | 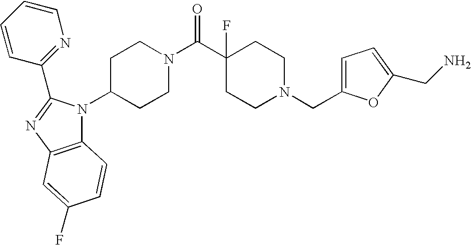 | 535 (ESMS) |
| 393 | 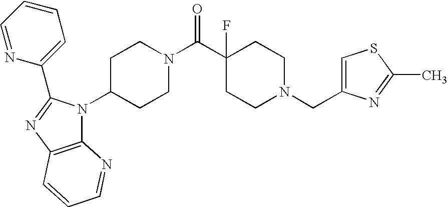 | 520 (ESMS) |
| 394 | 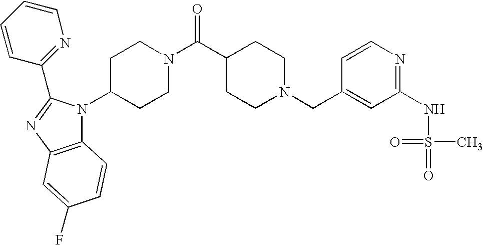 | 592 (FAB) |
| 395 | 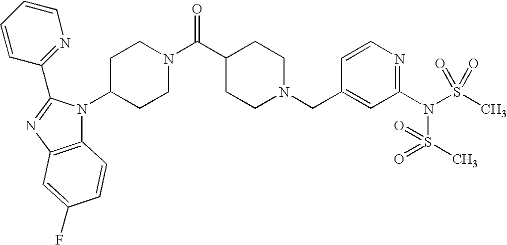 | 670 (FAB) |
| 396 | 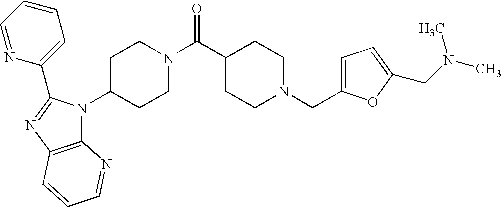 | 528 (ESMS) |
| 397 | 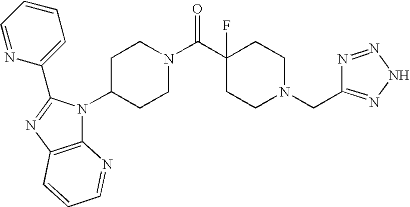 | 491 (ESMS) |
| 398 | 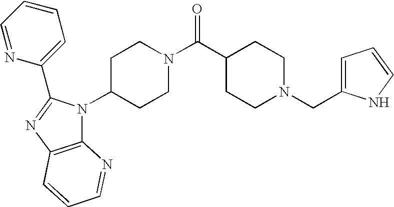 | 470 (ESMS) |
| 399 | 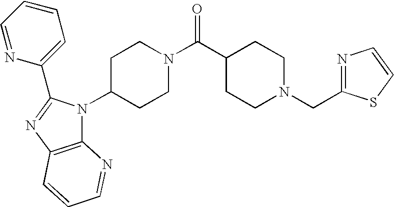 | 488 (ESMS) |
| 400 | 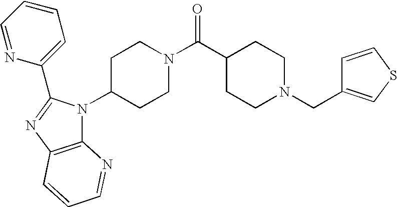 | 487 (ESMS) |
| 401 | 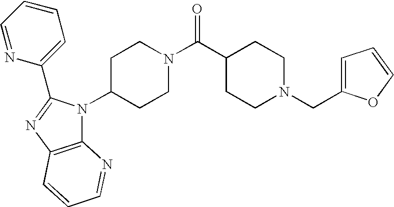 | 471 (ESMS) |
| 402 | 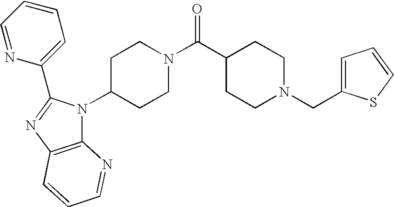 | 487 (ESMS) |
| 403 | 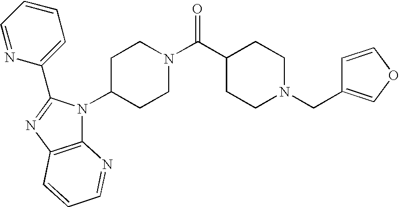 | 471 (ESMS) |
| 404 | 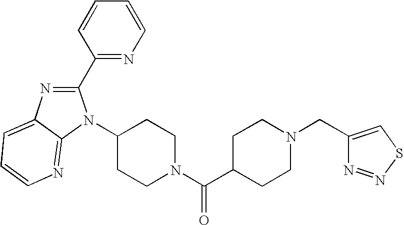 | 489 (ESMS) |
| 405 | 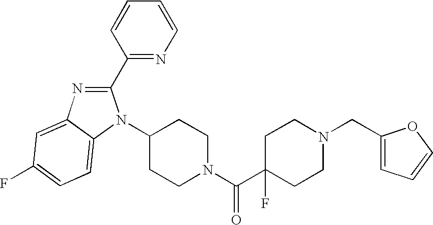 | 506 (ESMS) |
| 406 | 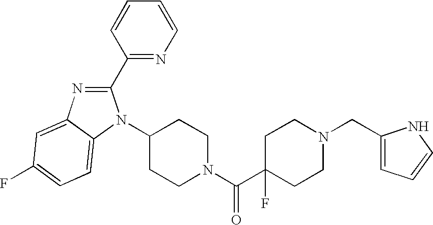 | 505 (ESMS) |
| 407 | 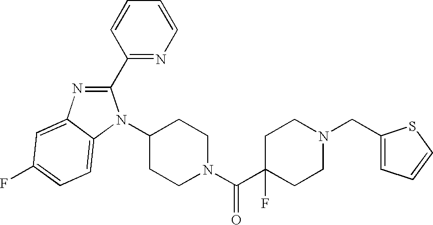 | 522 (ESMS) |
| 408 | 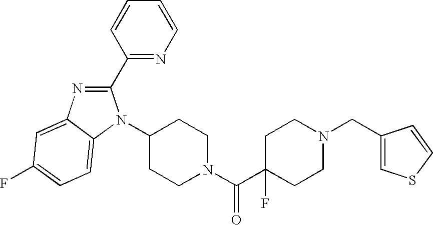 | 522 (ESMS) |
| 409 | 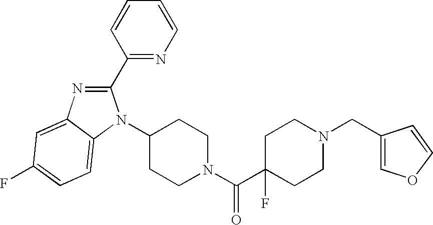 | 506 (ESMS) |
| 410 | 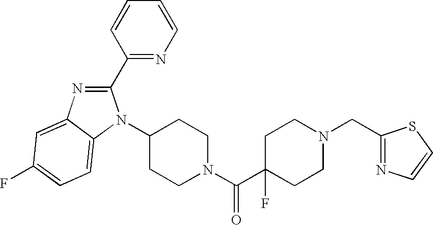 | 523 (ESMS) |
| 411 | 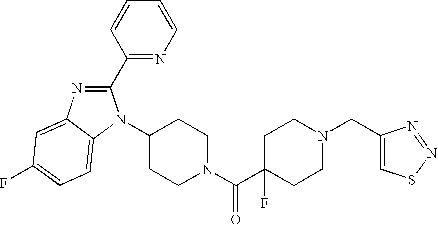 | 524 (ESMS) |
| 412 | 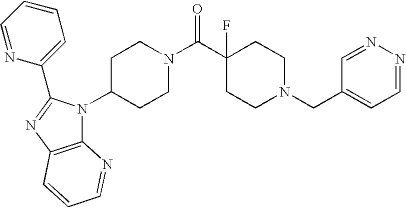 | 501 (ESMS) |
| 413 | 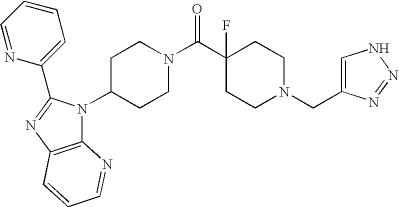 | 490 (ESMS) |
| 414 | 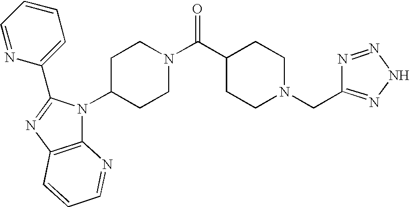 | 473 (ESMS) |
| 415 | 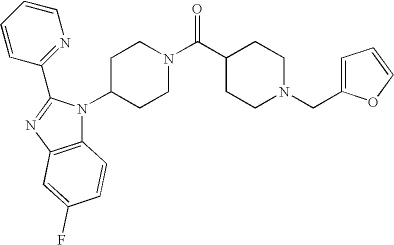 | 488 (ESMS) |
| 416 | 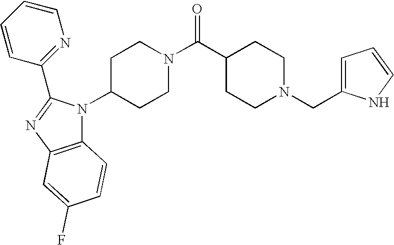 | 487 (ESMS) |
| 417 | 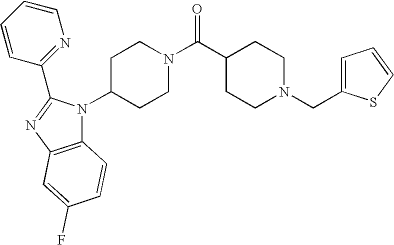 | 504 (ESMS) |
| 418 | 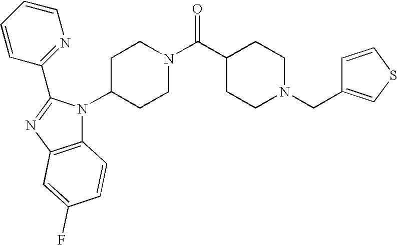 | 504 (ESMS) |
| 419 | 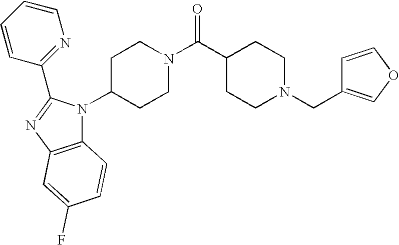 | 488 (ESMS) |
| 420 | 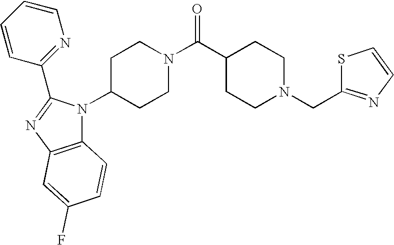 | 505 (ESMS) |
| 421 | 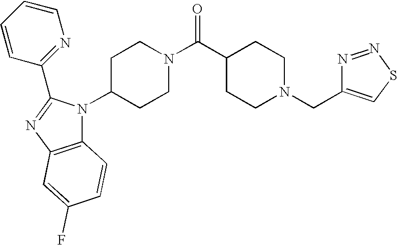 | 506 (ESMS) |
| 422 | 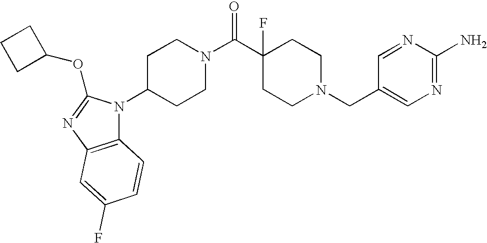 | 526 (FAB) |
| 423 | 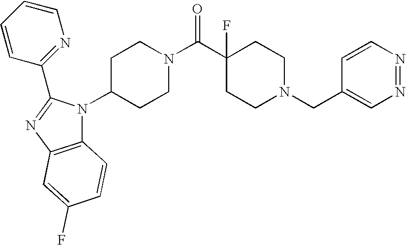 | 518 (ESMS) |
| 424 | 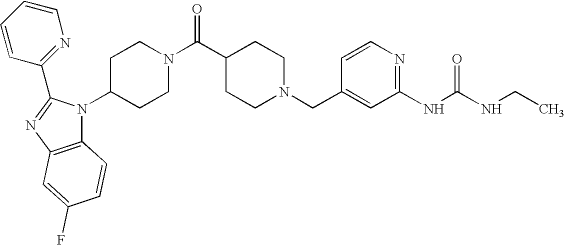 | 585 (FAB) |
| 425 | 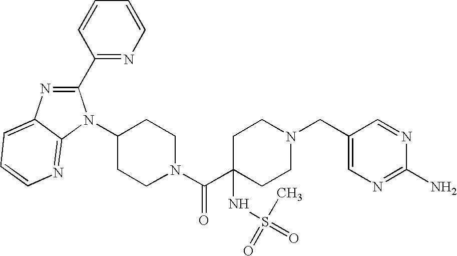 | 591 (ESMS) |
| 426 | 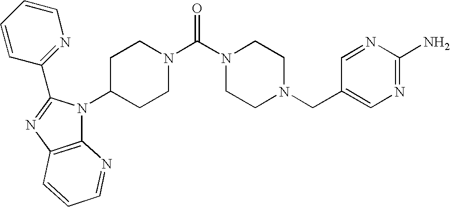 | 499 (ESMS) |
| 427 | 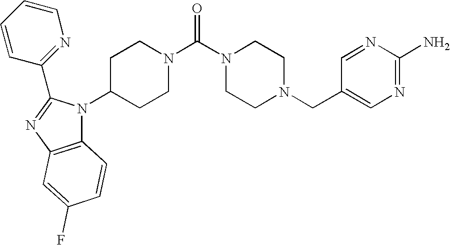 | 516 (ESMS) |
| 428 | 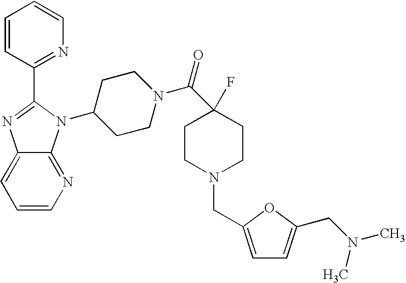 | 546 (ESMS) |
| 429 | 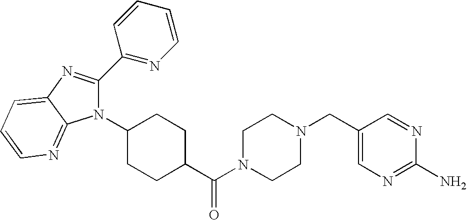 | 498 (ESMS) |
| 430 | 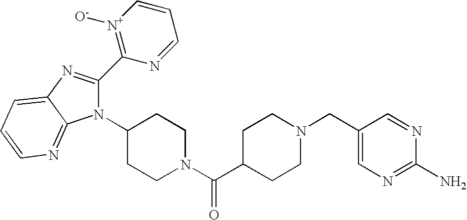 | 514 (ESMS) |
| 431 | 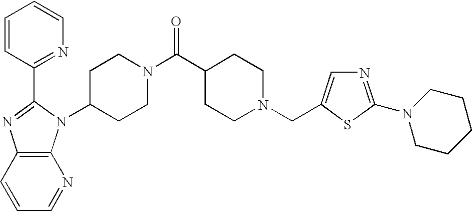 | 571 (ESMS) |
| 432 | 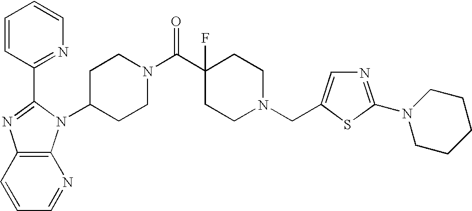 | 589 (ESMS) |
| 433 | 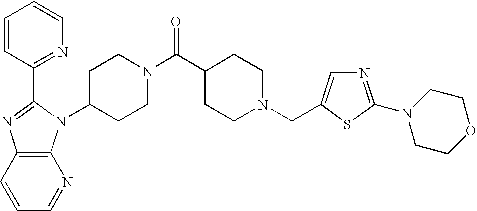 | 573 (ESMS) |
| 434 | 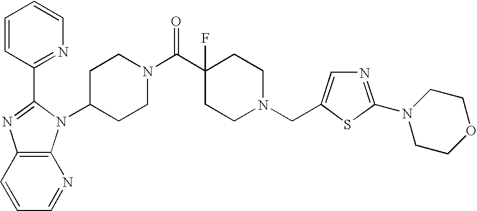 | 591 (ESMS) |
| 435 | 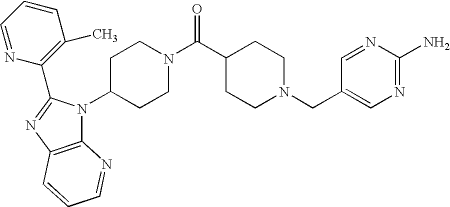 | 512 (ESMS) |
| 436 | 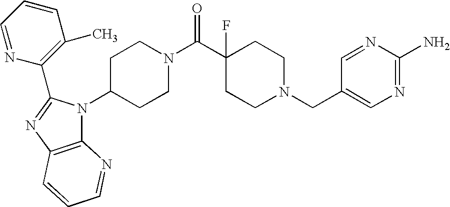 | 530 (ESMS) |
| 437 | 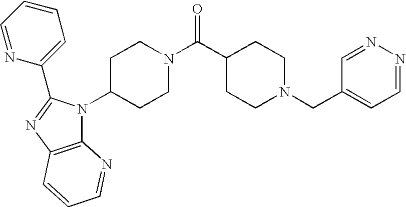 | 483 (ESMS) |
| 438 | 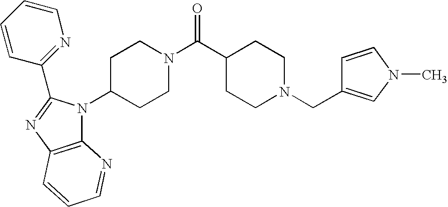 | 484 (ESMS) |
| 439 | 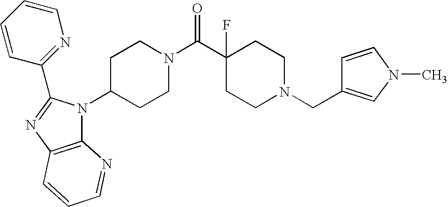 | 502 (ESMS) |
| 440 | 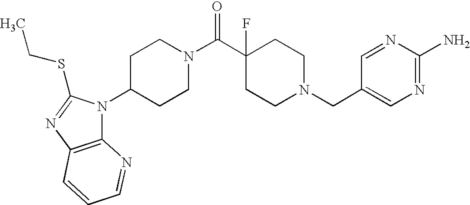 | 499 (FAB) |
| 441 | 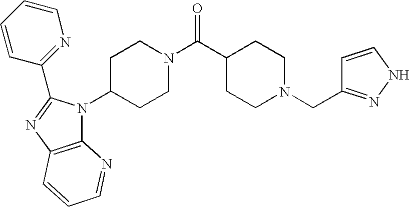 | 471 (ESMS) |
| 442 | 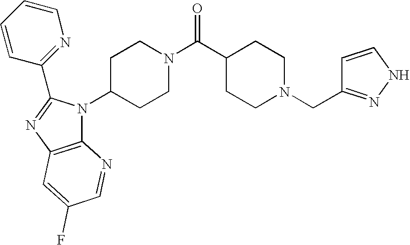 | 488 (ESMS) |
| 443 | 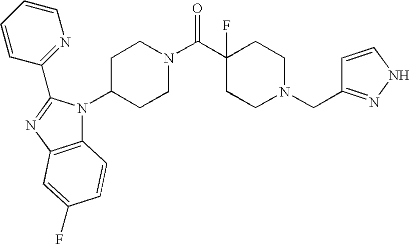 | 506 (ESMS) |
| 444 | 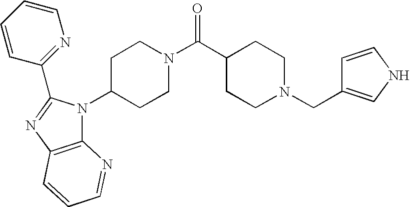 | 470 (ESMS) |
| 445 | 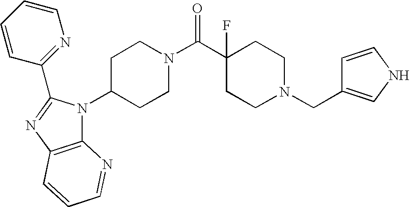 | 488 (ESMS) |
| 446 | 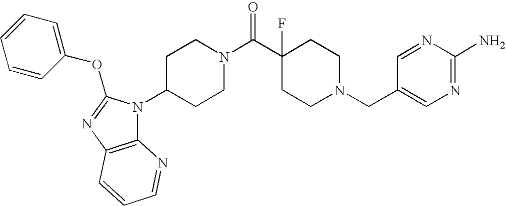 | 531 (FAB) |
| 447 | 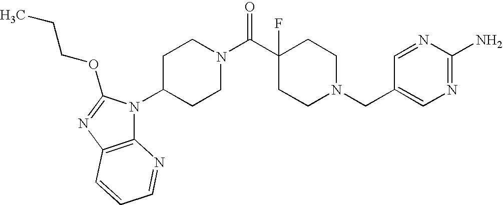 | 497 (FAB) |
| 448 | 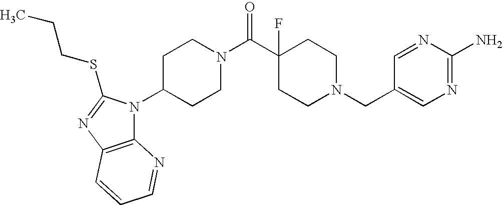 | 513 (FAB) |
| 449 | 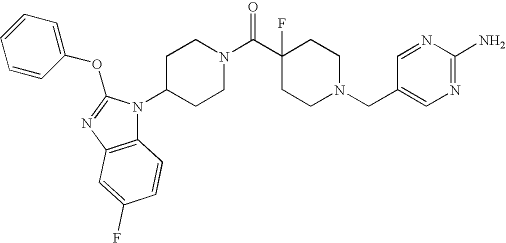 | 548 (FAB) |
| 450 | 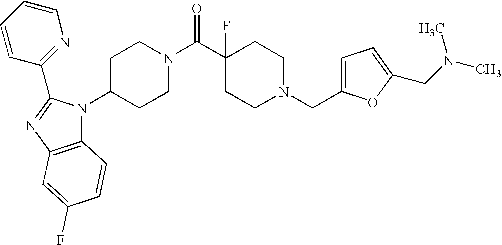 | 563 (ESMS) |
| 451 | 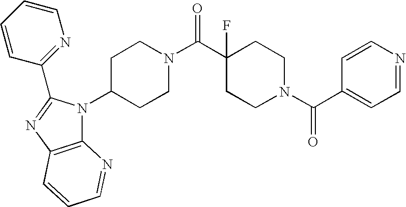 | 514 (ESMS) |
| 452 | 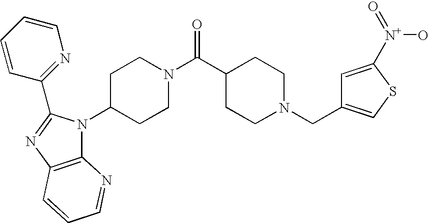 | 532 (ESMS) |
| 453 | 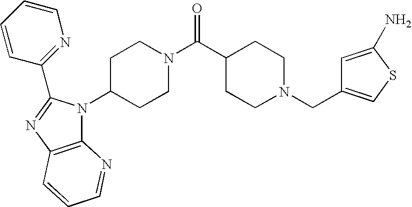 | 502 (ESMS) |
| 454 | 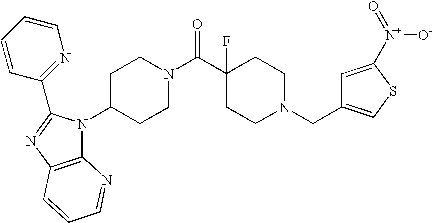 | 550 (ESMS) |
| 455 | 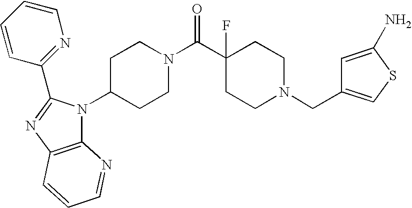 | 520 (ESMS) |
| 456 | 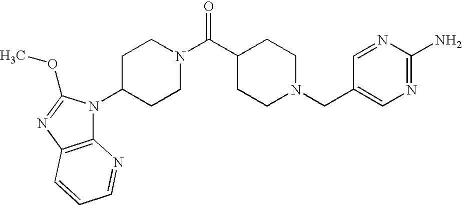 | 451 (ESMA) |
| 457 | 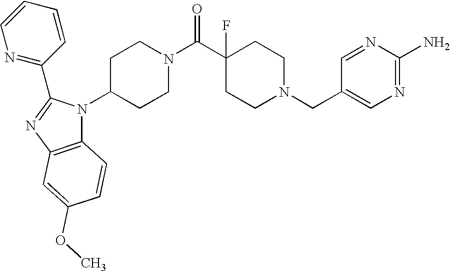 | 545 (ESMS) |
| 458 | 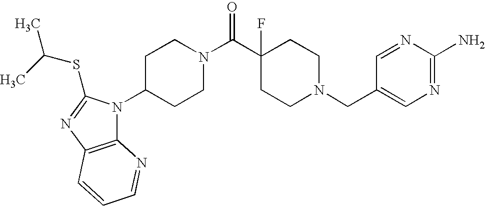 | 513 (ESMS) |
| 459 | 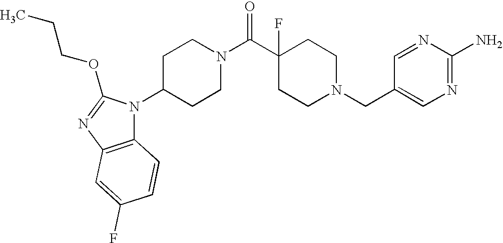 | 514 (FAB) |
| 460 | 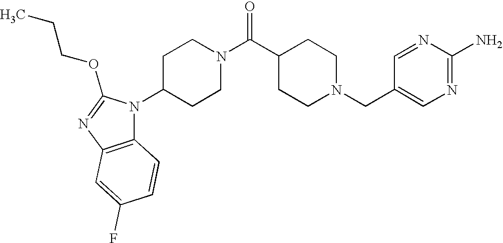 | 496 (FAB) |
| 461 | 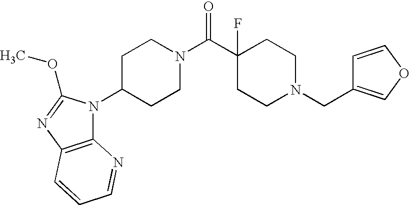 | 442 (ESMS) |
| 462 | 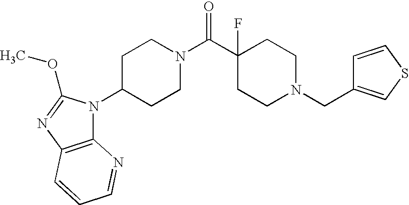 | 458 (ESMS) |
| 463 | 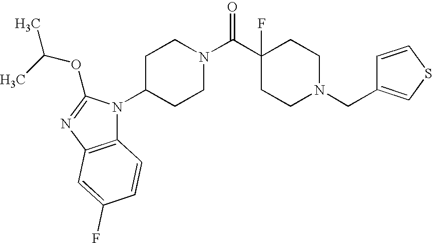 | 503 (ESMS) |
| 464 | 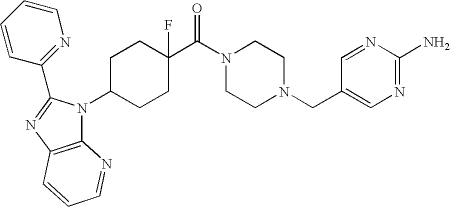 | 407 (ESMS) |
| 465 | 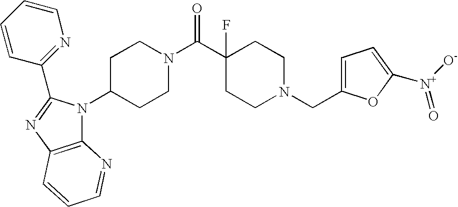 | 534 (ESMS) |
| 466 | 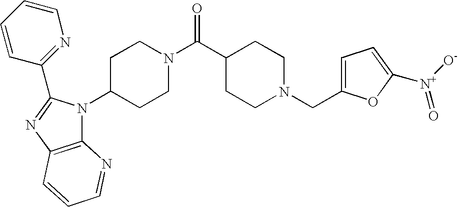 | 516 (ESMS) |
| 467 | 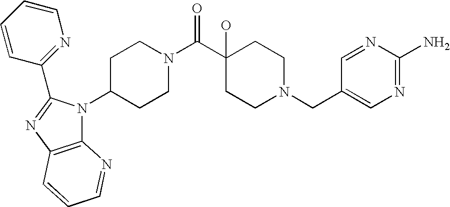 | 514 (ESMS) |
| 468 | 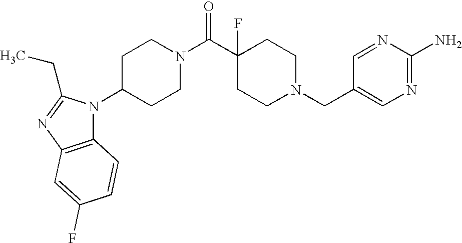 | 484 (ESMS) |
| 469 | 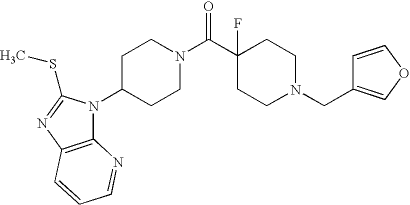 | 458 (ESMS) |
| 470 | 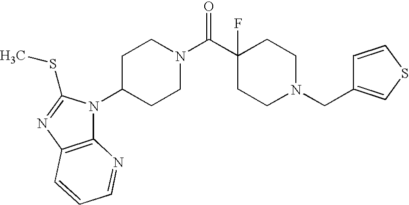 | 474 (ESMS) |
| 471 | 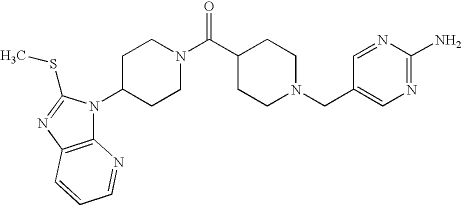 | 467 (ESMA) |
| 472 | 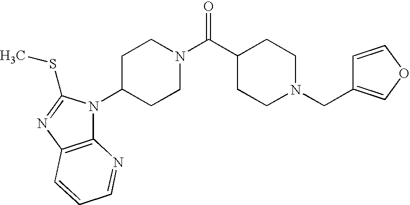 | 440 (ESMS) |
| 473 | 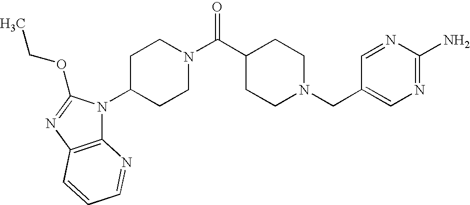 | 465 (ESMS) |
| 474 | 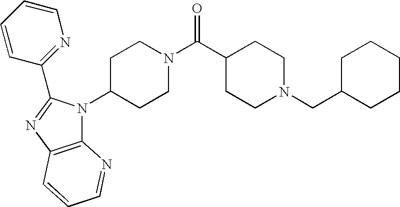 | 487 (ESMS) |
| 475 | 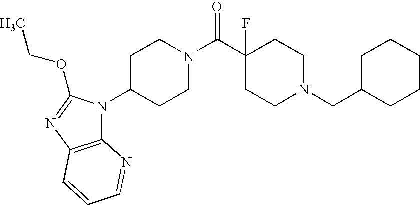 | 472 (ESMS) |
| 476 | 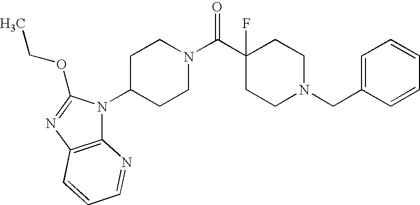 | 466 (ESMS) |
| 477 | 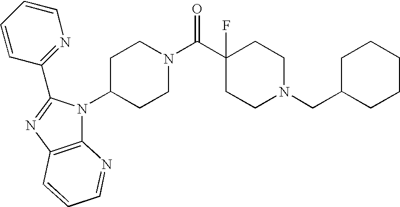 | 505 (ESMS) |
| 478 | 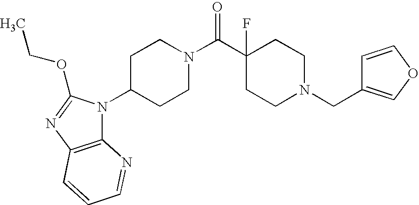 | 456 (ESMS) |
| 479 | 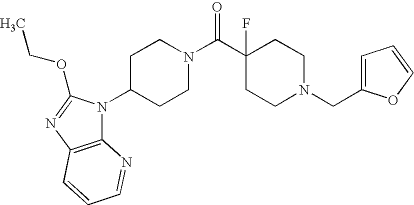 | 456 (ESMS) |
| 480 | 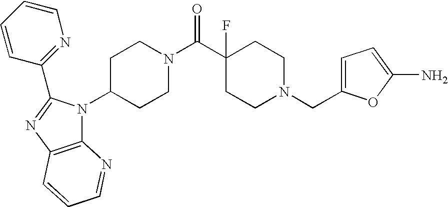 | 504 (ESMS) |
| 481 | 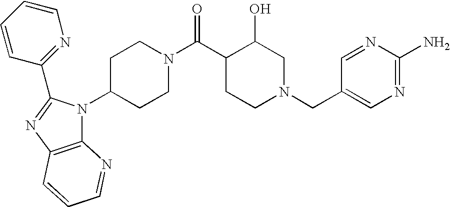 | 514 (ESMS) |
| 482 | 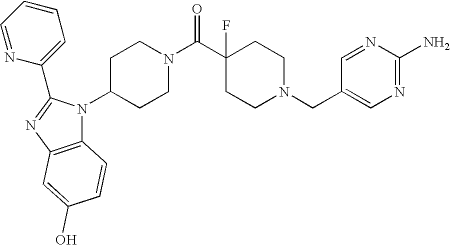 | 531 (FAB) |
| 483 | 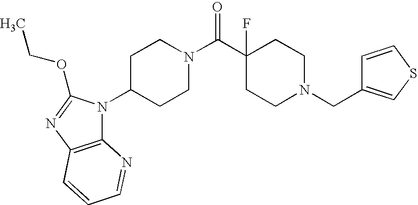 | 472 (ESMS) |
| 484 | 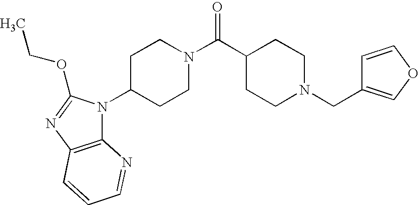 | 438 (ESMS) |
| 485 | 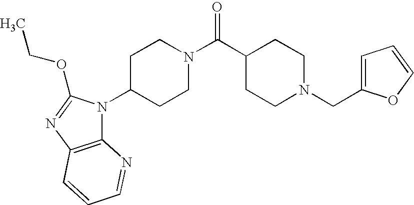 | 438 (ESMS) |
| 486 | 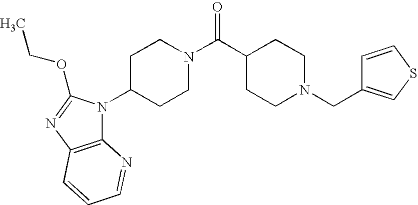 | 454 (ESMS) |
| 487 | 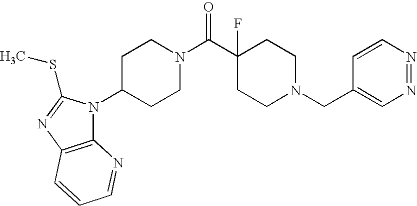 | 470 (ESMS) |
| 488 | 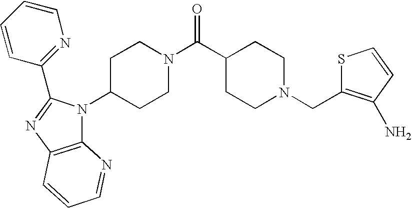 | 502 (ESMS) |
| 489 | 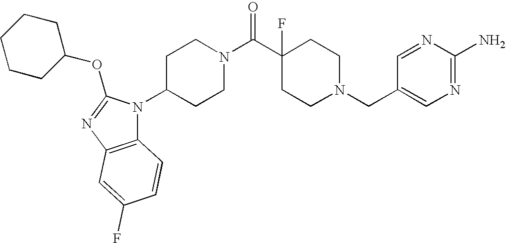 | 554 (FAB) |
| 490 | 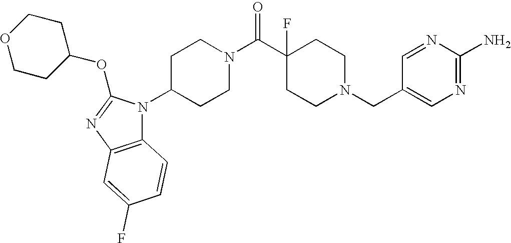 | 556 (FAB) |
| 491 | 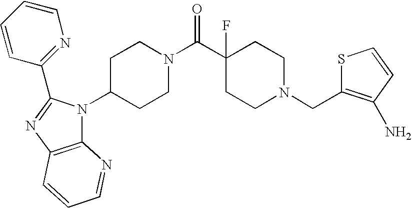 | 470 (ESMS) |
| 492 | 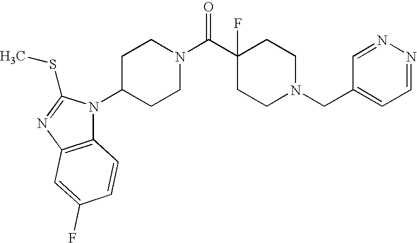 | 487 (ESMS) |
| 493 | 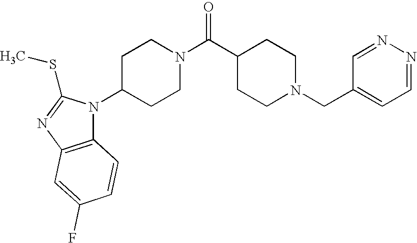 | 469 (ESMS) |
| 44 | 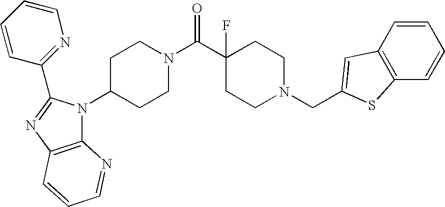 | 555 (ESMS) |
| 495 | 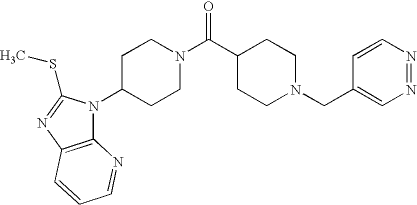 | 452 (ESMS) |
| 496 | 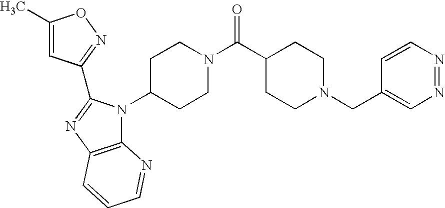 | 487 (ESMS) |
| 497 | 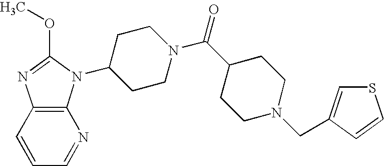 | 440 (ESMS) |
| 498 | 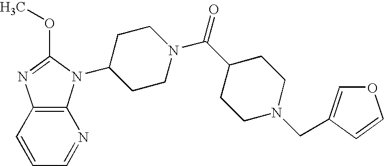 | 424 (ESMS) |
| 499 | 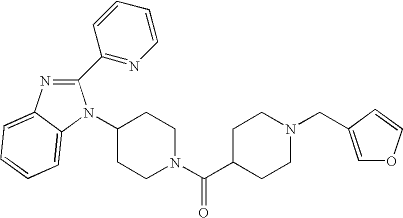 | 470 (ESMS) |
| 500 | 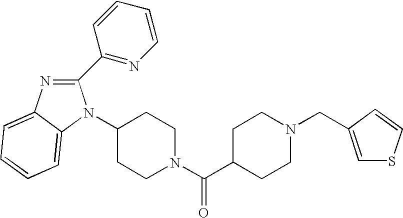 | 486 (ESMS) |
| 501 | 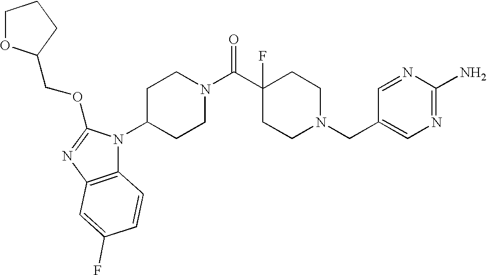 | 556 (ESMS) |
| 502 | 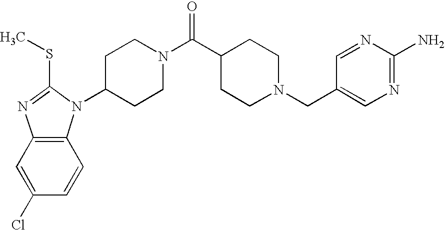 | 500 (ESMS) |
| 503 | 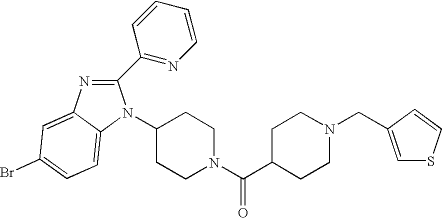 | 566 (ESMS) |
| 504 | 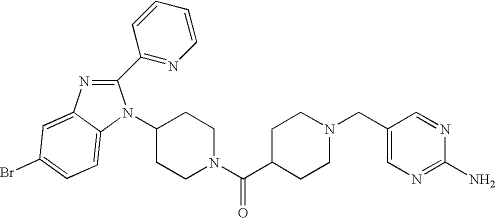 | 577 (ESMS) |
| 505 | 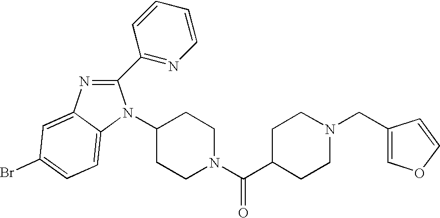 | 550 (ESMS) |
| 506 | 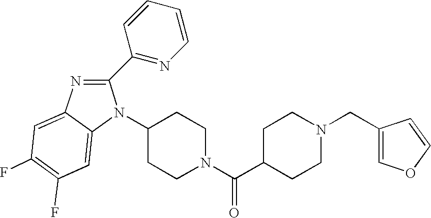 | 506 (ESMS) |
| 507 | 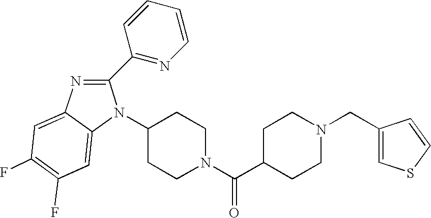 | 522 (ESMS) |
| 508 | 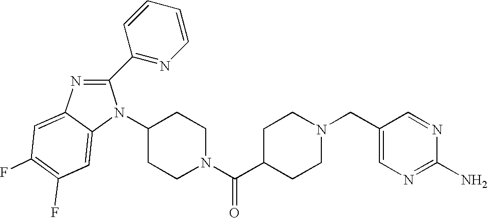 | 533 (ESMS) |
| 509 | 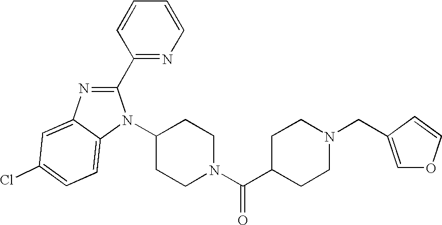 | 504 (ESMS) |
| 510 | 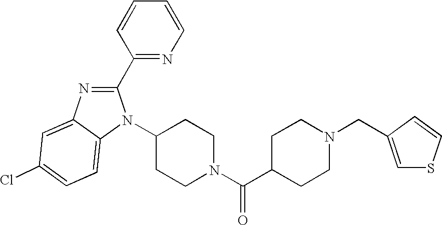 | 520 (ESMS) |
| 511 | 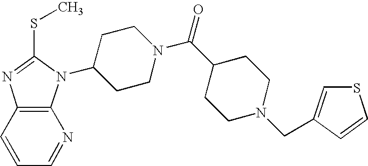 | 456 (ESMS) |
| 512 | 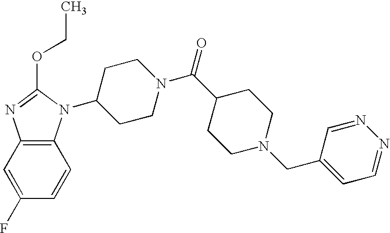 | 467 (ESMS) |
| 513 | 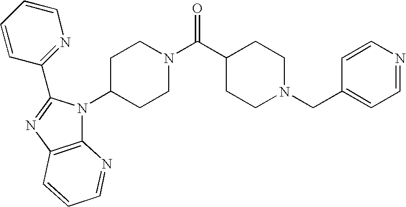 | 482 (ESMS) |
| 514 | 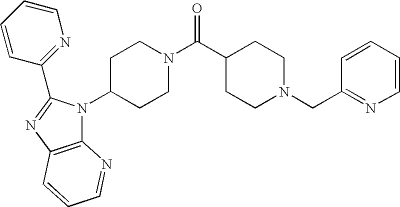 | 482 (ESMS) |
| 515 | 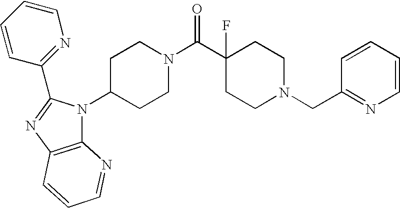 | 500 (ESMS) |
| 516 | 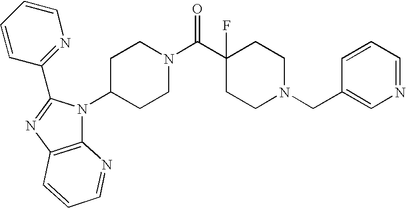 | 500 (ESMS) |
| 517 | 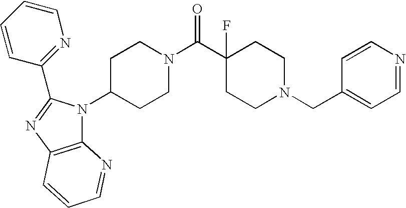 | 500 (ESMS) |
| 518 | 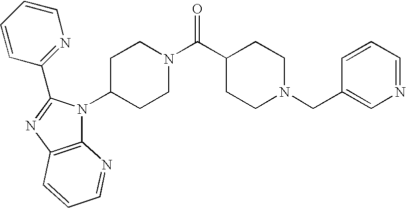 | 482 (ESMS) |
| 519 | 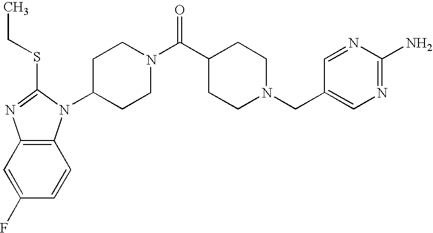 | 498 (ESMS) |
| 520 | 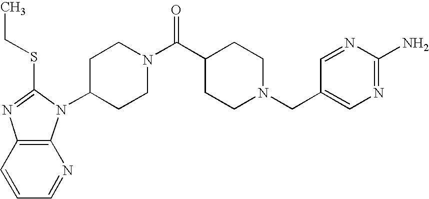 | 481 (ESMS) |
| 521 | 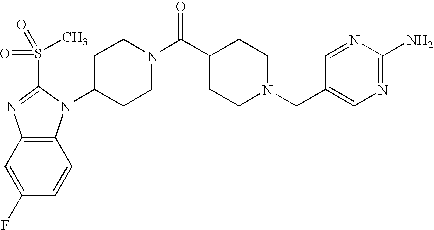 | 516 (ESMS) |
| 522 | 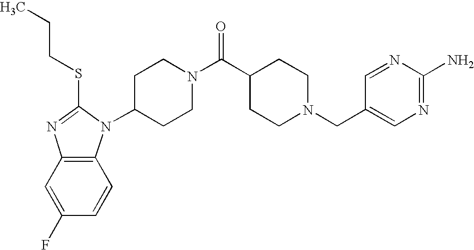 | 512 (FAB) |
| 523 | 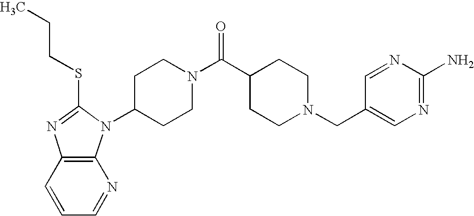 | 495 (FAB) |
| 524 | 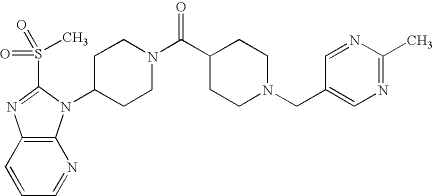 | 499 (FAB) |
| 525 | 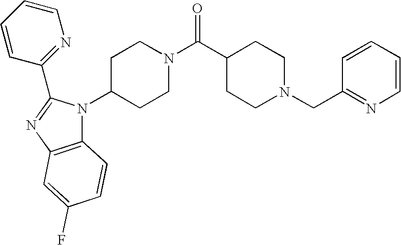 | 499 (ESMS) |
| 526 | 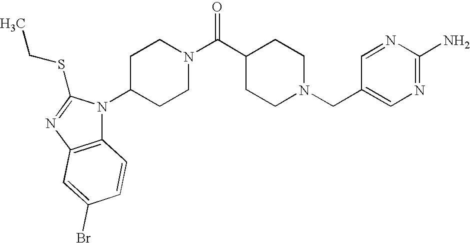 | 560 (ESMS) |
| 527 | 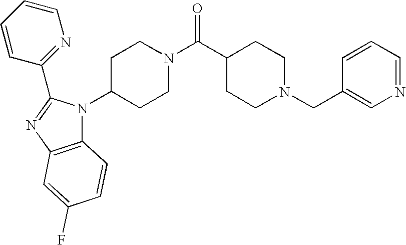 | 499 (ESMS) |
| 528 | 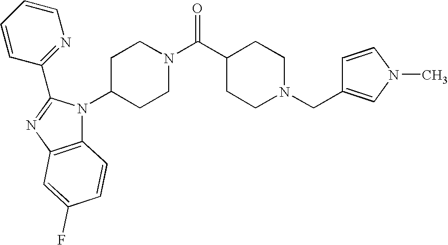 | 501 (ESMS) |
| 529 | 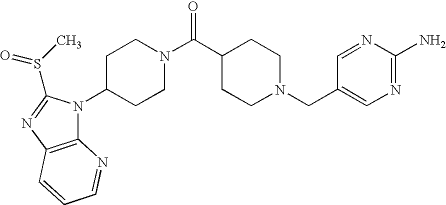 | 483 (ESMS) |
| 530 | 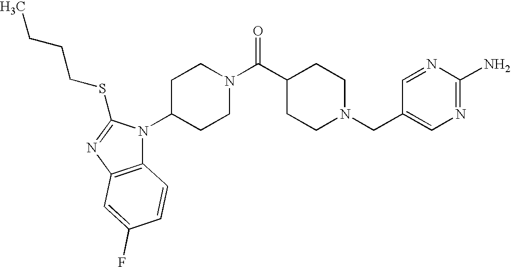 | 526 (ESMS) |
| 531 | 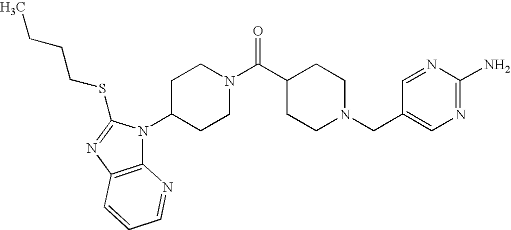 | 509 (ESMS) |
| 532 | 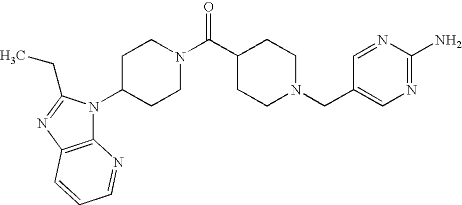 | 449 (ESMS) |
| 533 | 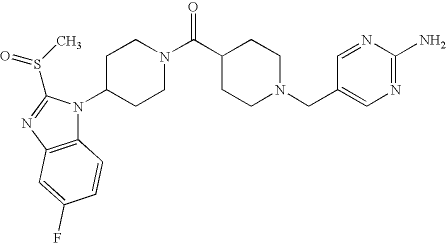 | 500 (ESMS) |
| 534 | 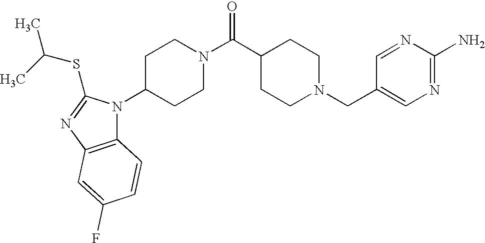 | 512 (ESMS) |
| 535 | 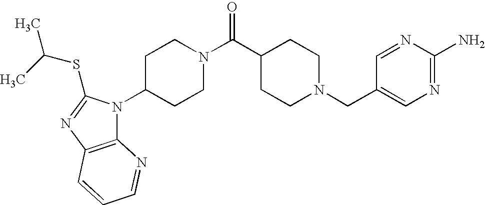 | 495 (ESMS) |
| 536 | 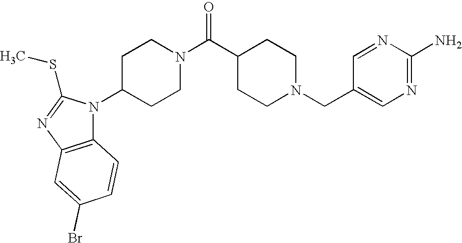 | 546 (ESMS) |
| 537 | 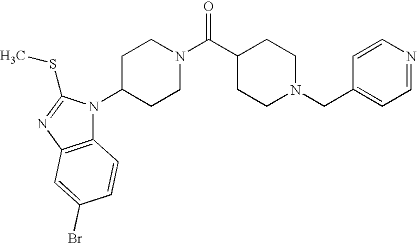 | 530 (ESMS) |
| 538 | 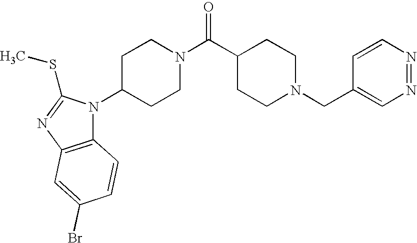 | 531 (ESMS) |
| 539 | 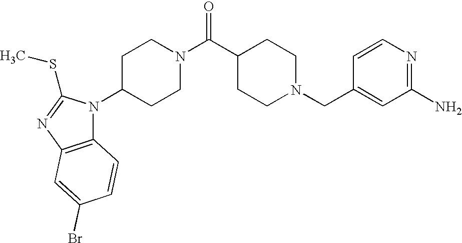 | 545 (ESMS) |
| 540 | 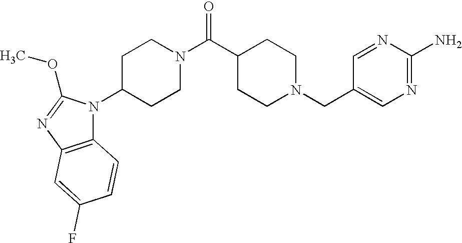 | 468 (ESMS) |
| 541 | 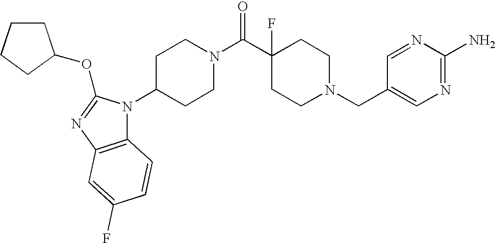 | 540 (ESMS) |
| 542 | 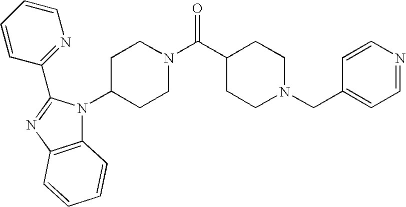 | 481 (ESMS) |
| 543 | 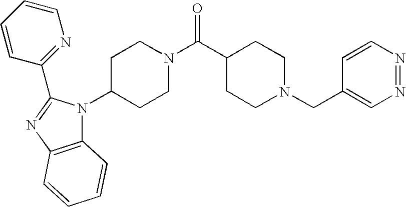 | 482 (ESMS) |
| 544 | 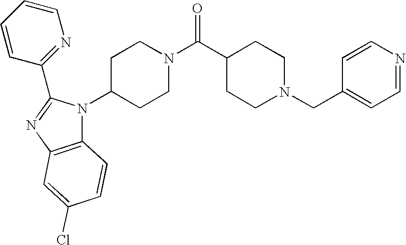 | 515 (ESMS) |
| 545 | 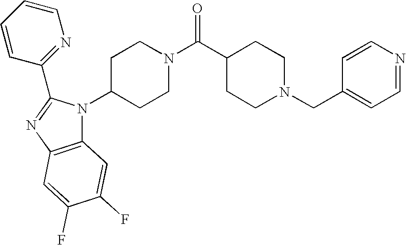 | 517 (ESMS) |
| 546 | 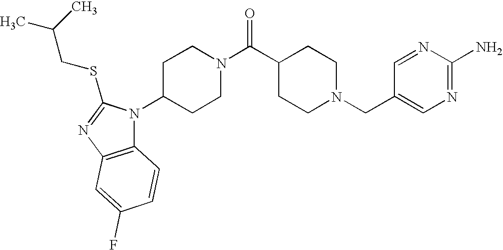 | 526 (ESMS) |
| 547 | 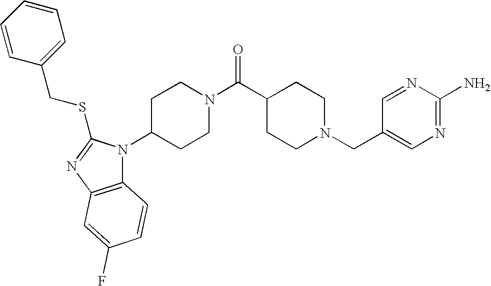 | 5560 (ESMS) |
| 548 | 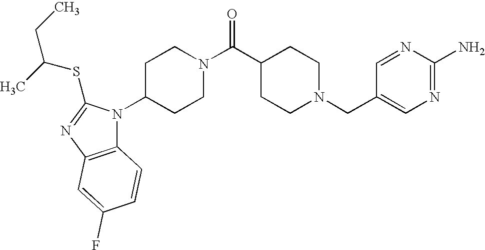 | 526 (ESMS) |
| 549 | 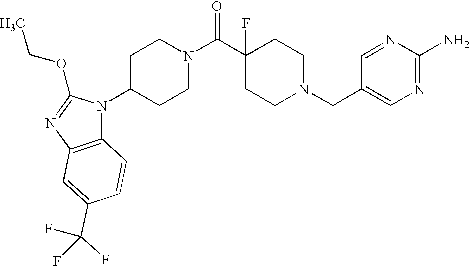 | 550 (ESMS) |
| 550 | 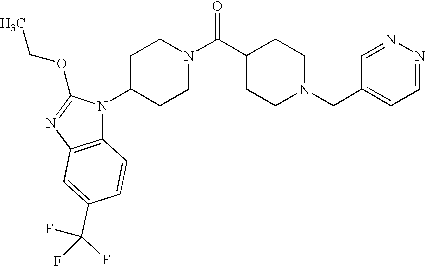 | 517 (ESMS) |
| 551 | 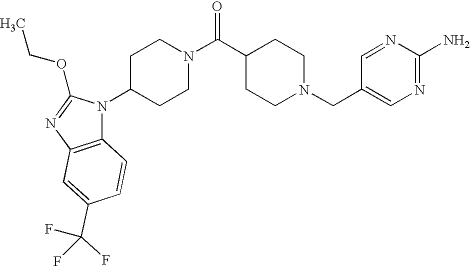 | 532 (ESMS) |
| 552 | 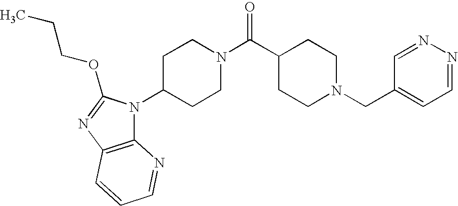 | 464 (ESMS) |
| 553 | 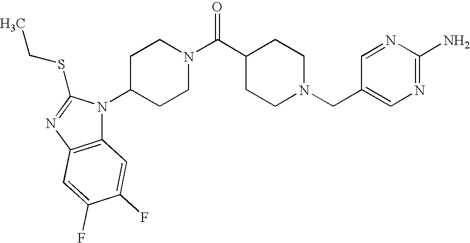 | 516 (ESMS) |
| 554 | 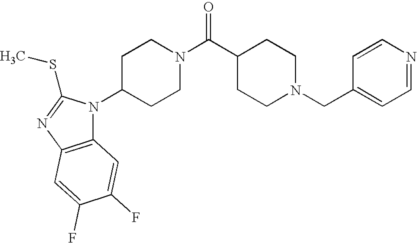 | 486 (ESMS) |
| 555 | 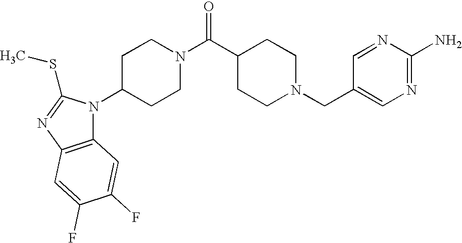 | 502 (ESMS) |
| 556 | 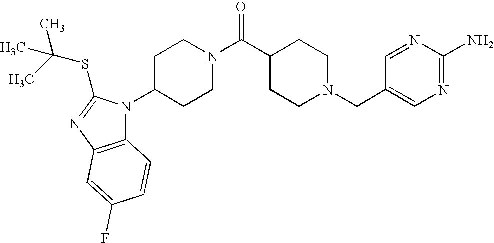 | 526 (ESMS) |
| 557 | 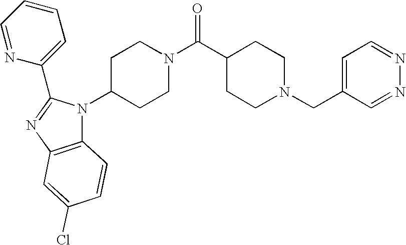 | 516 (ESMS) |
| 558 | 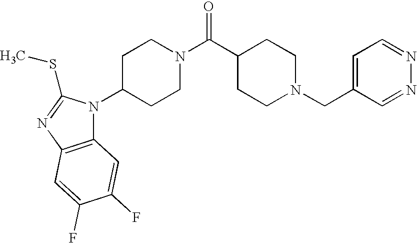 | 487 (ESMS) |
| 559 | 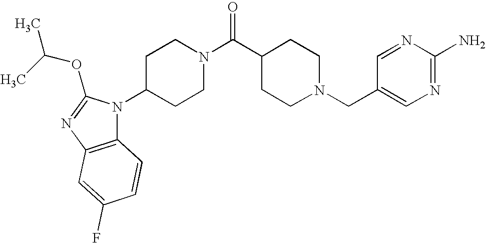 | 496 (ESMS) |
| 560 | 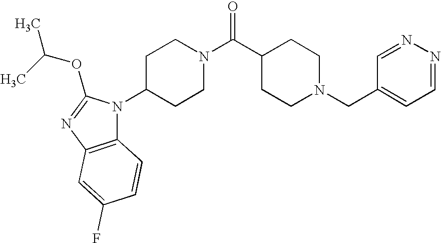 | 481 (FAB) |
| 561 | 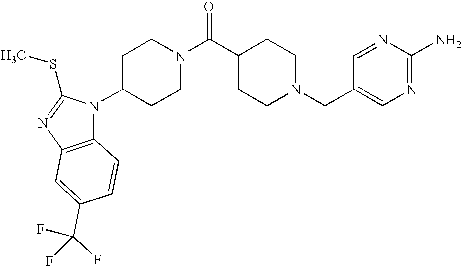 | 534 (ESMS) |
| 562 | 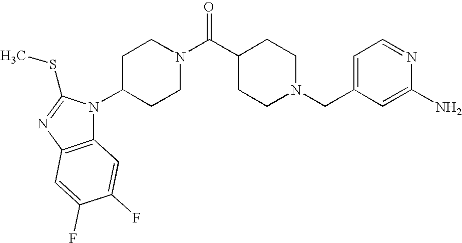 | 501 (ESMS) |
| 563 | 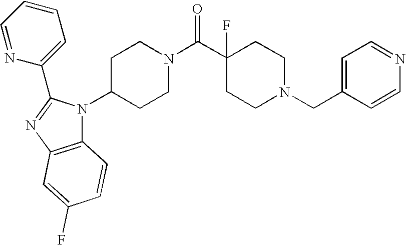 | 517 (ESMS) |
| 564 | 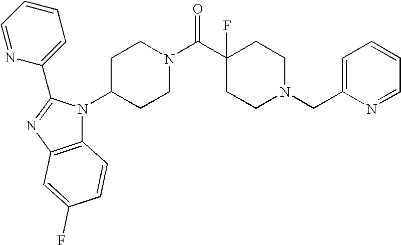 | 517 (ESMS) |
| 565 | 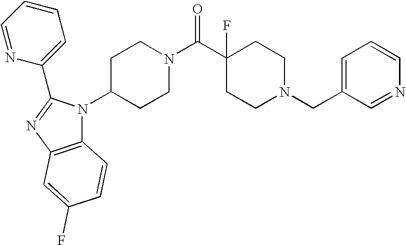 | 517 (ESMS) |
| 566 | 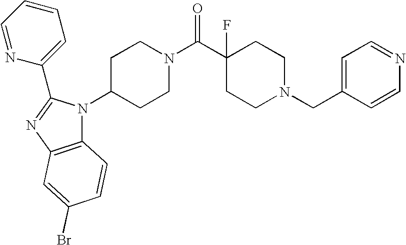 | 577 (ESMS) |
| 567 | 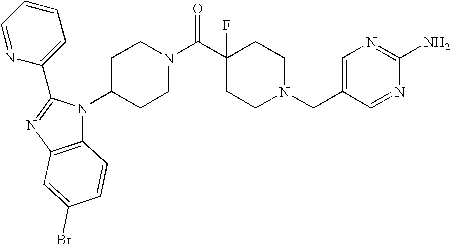 | 592 (ESMS) |
| 568 | 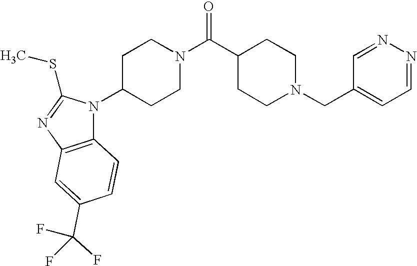 | 519 (ESMS) |
| 569 | 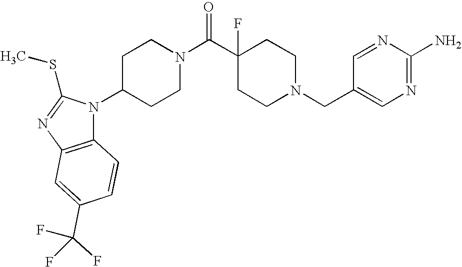 | 552 (ESMS) |
| 570 | 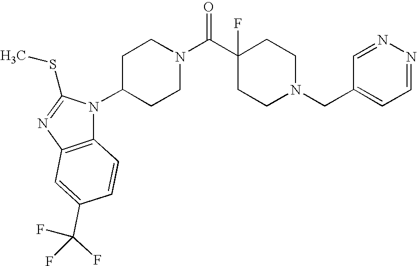 | 537 (ESMS) |
| 571 | 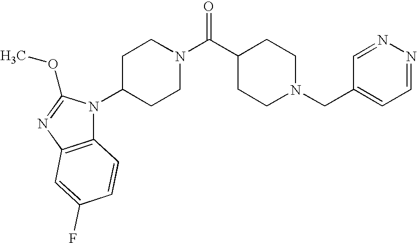 | 453 (ESMS) |
| 572 | 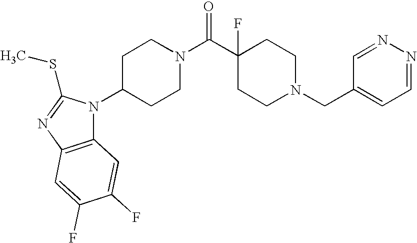 | 505 (ESMS) |
| 573 | 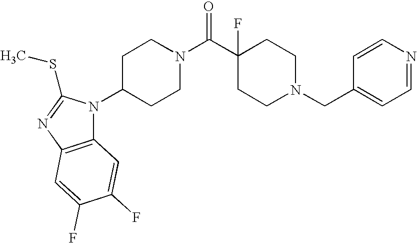 | 504 (ESMS) |
| 574 | 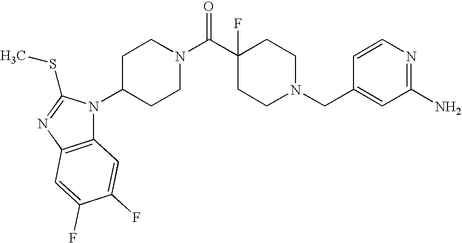 | 519 (ESMS) |
| 575 | 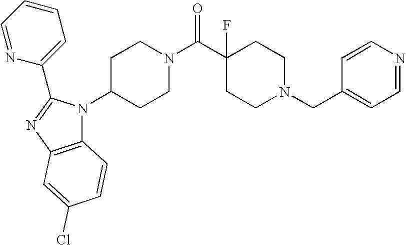 | 533 (ESMS) |
| 576 | 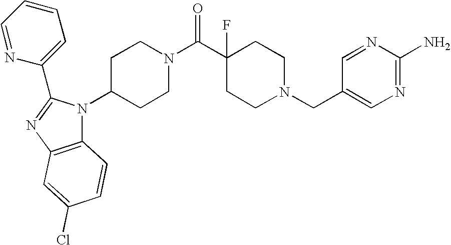 | 549 (ESMS) |
| 577 | 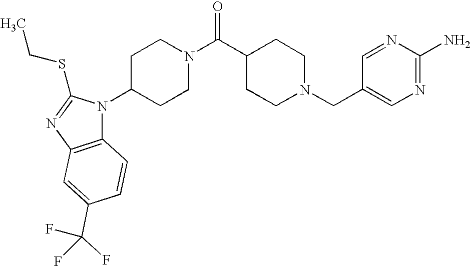 | 548 (ESMS) |
| 578 | 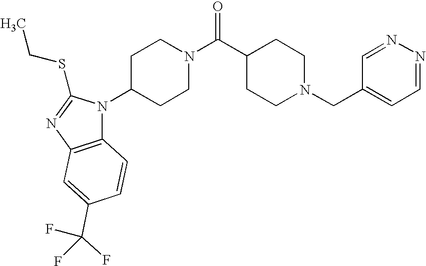 | 533 (ESMS) |
| 579 | 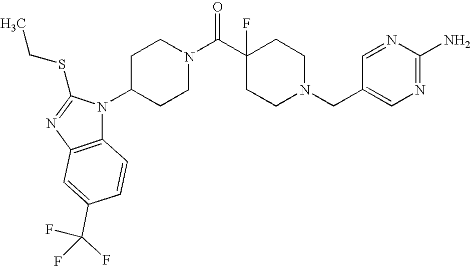 | 566 (ESMS) |
| 580 | 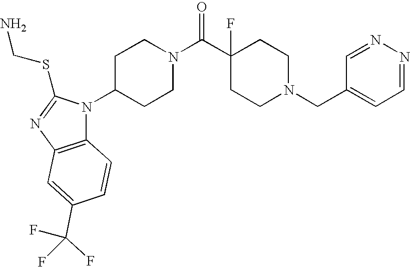 | 551 (ESMS) |
| 581 | 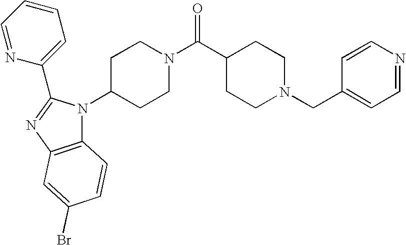 | 559 (ESMS) |
| 582 | 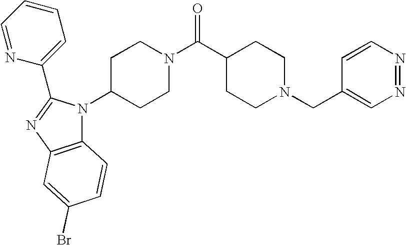 | 560 (ESMS) |
| 583 | 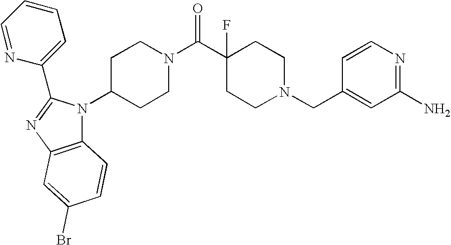 | 592 (ESMS) |
| 584 | 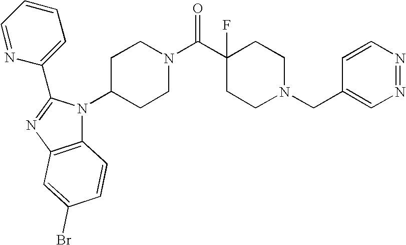 | 579 (ESMS) |
| 585 | 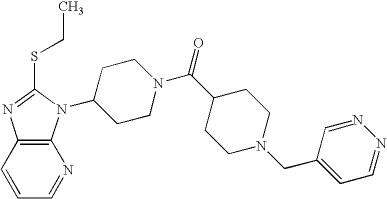 | 466 (ESMS) |
| 586 | 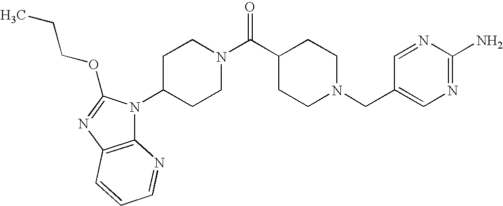 | 479 (FAB) |
| 587 | 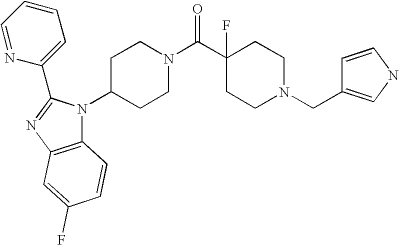 | 505 (ESMS) |
| 588 | 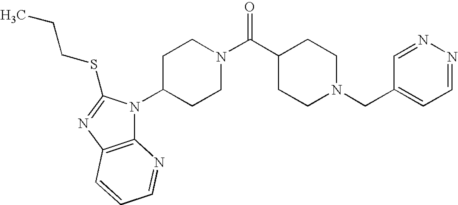 | 480 (ESMS) |
| 589 | 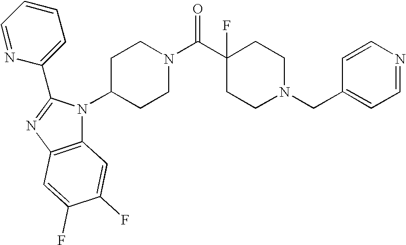 | 535 (ESMS) |
| 590 | 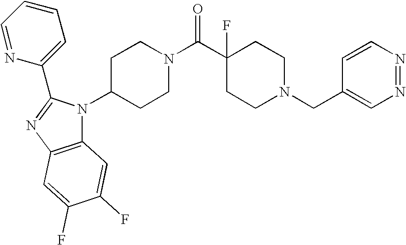 | 536 (ESMS) |
| 591 | 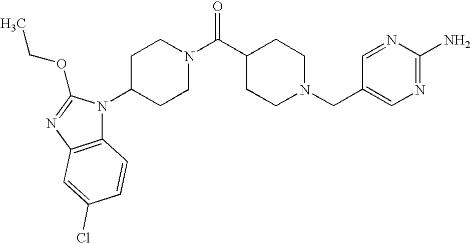 | 498 (ESMS) |
| 592 | 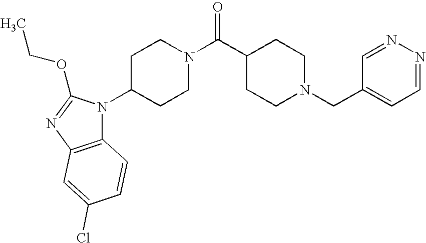 | 483 (ESMS) |
| 593 | 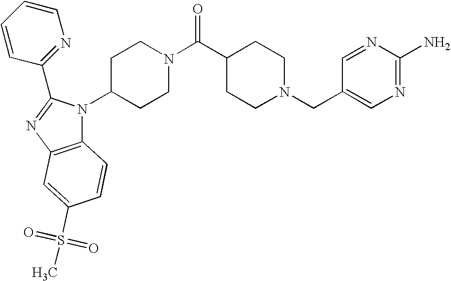 | 575 (ESMS) |
| 594 | 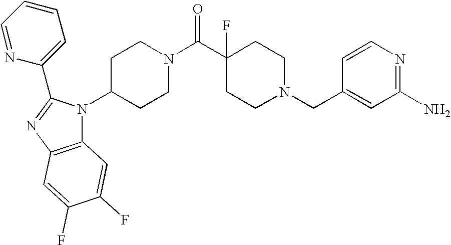 | 550 (ESMS) |
| 595 | 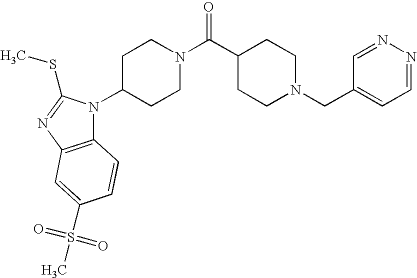 | 529 (ESMS) |
| 596 | 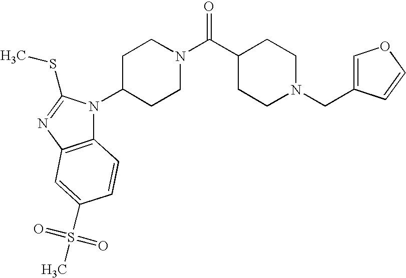 | 517 (ESMS) |
| 597 | 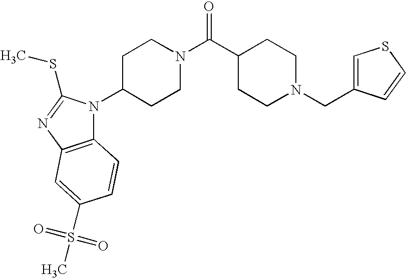 | 533 (ESMS) |
| 598 | 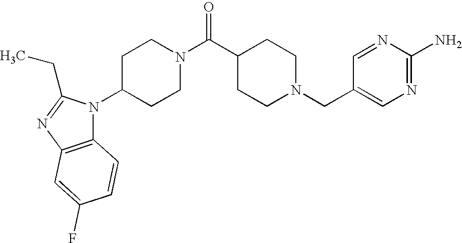 | 466 (ESMS) |
| 599 | 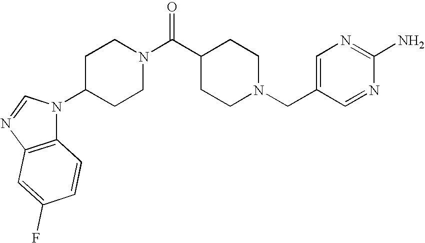 | 438 (ESMS) |
| 600 | 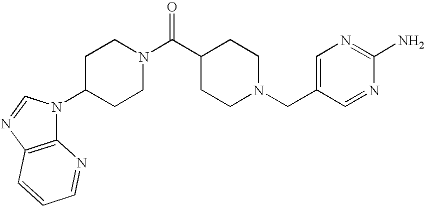 | 421 (ESMS) |
| 601 | 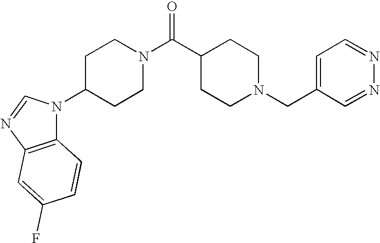 | 423 (ESMS) |
| 602 | 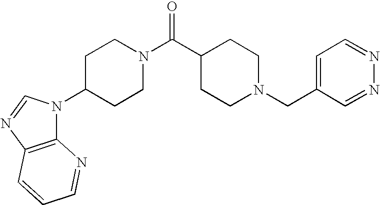 | 406 (ESMS) |
| 603 | 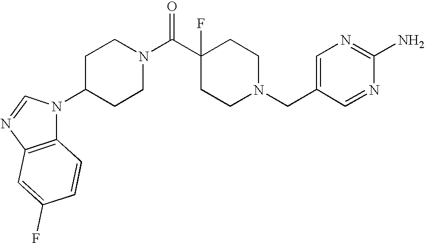 | 456 (ESMS) |
| 604 | 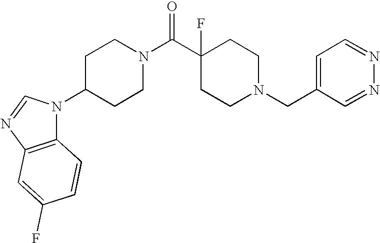 | 441 (ESMS) |
| 605 | 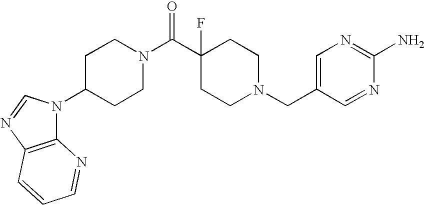 | 439 (ESMS) |
| 606 | 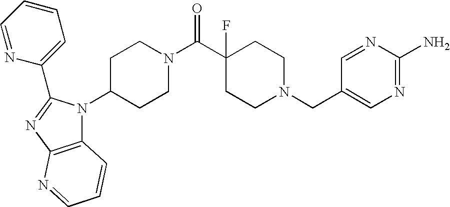 | 516 (ESMS) |
| 607 | 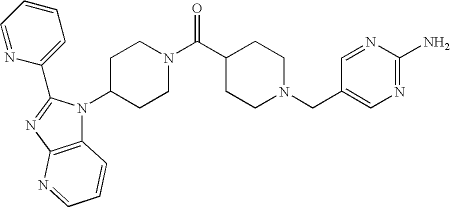 | 498 (ESMS) |
| 608 | 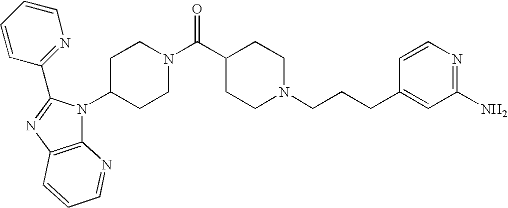 | 525 (ESMS) |
| 609 | 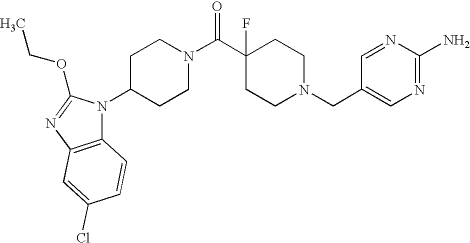 | 516 (ESMS) |
| 610 | 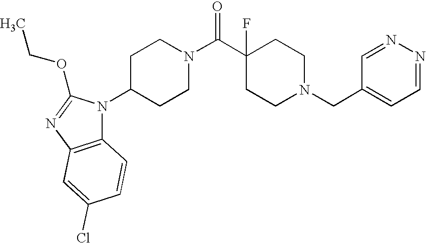 | 501 (ESMS) |
| 611 | 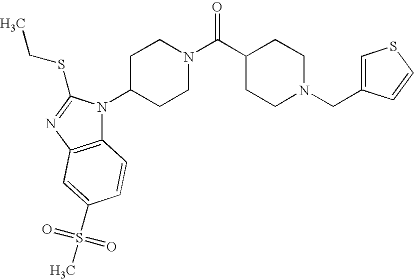 | 547 (ESMS) |
| 612 | 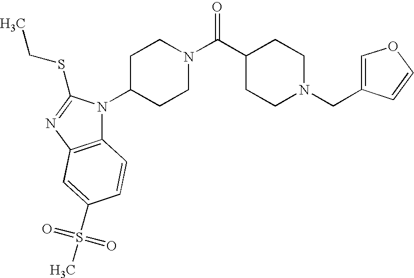 | 531 (ESMS) |
| 613 | 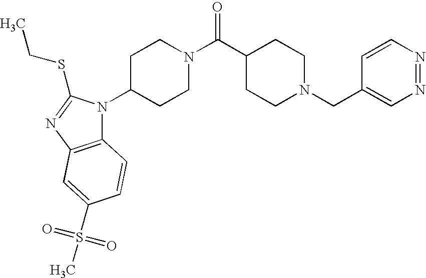 | 543 (ESMS) |
| 614 | 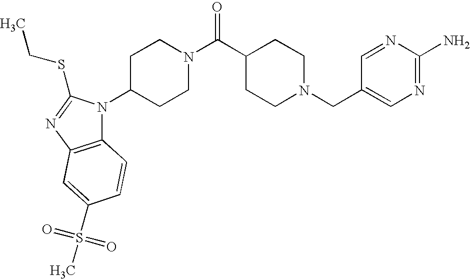 | 558 (ESMS) |
| 615 | 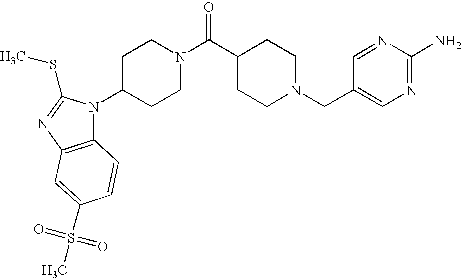 | 544 (ESMS) |
| 616 | 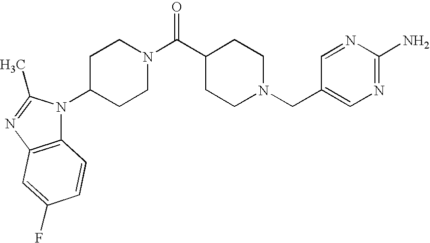 | 452 (FAB) |
| 617 | 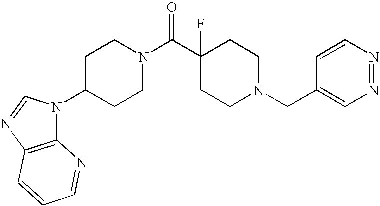 | 424 (ESMS) |
| 618 | 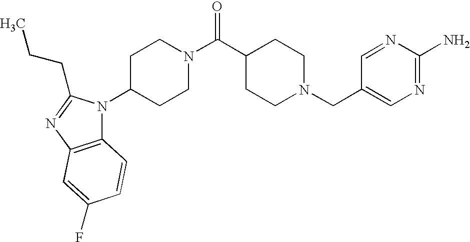 | 480 (ESMS) |
| 619 | 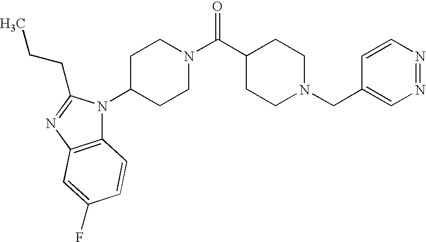 | 465 (ESMS) |
| 620 | 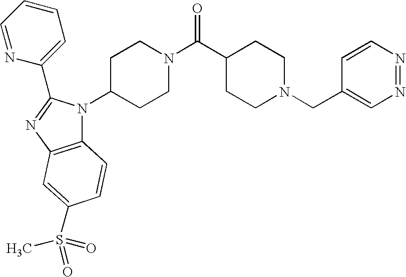 | 560 (ESMS) |
| 621 | 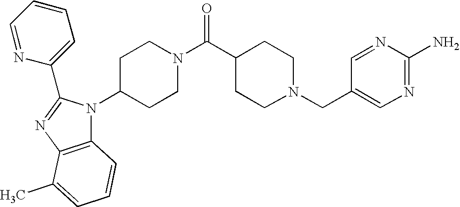 | 511 (ESMS) |
| 622 | 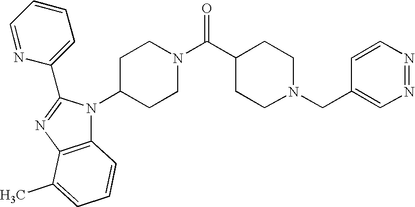 | 496 (ESMS) |
| 623 | 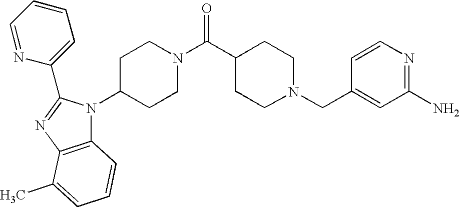 | 510 (ESMS) |
| 624 | 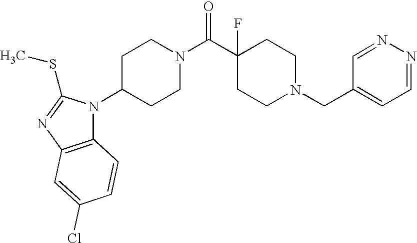 | 503 (ESMS) |
| 625 | 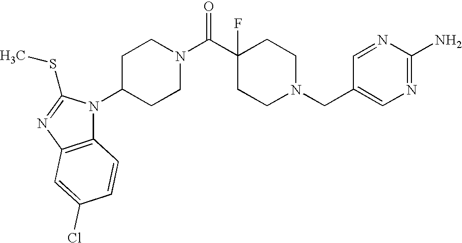 | 518 (ESMS) |
| 626 | 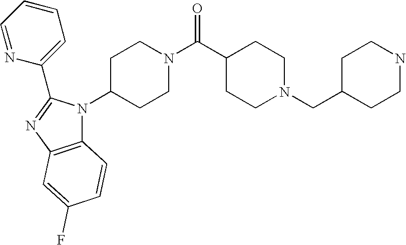 | 505 (ESMS) |
| 627 | 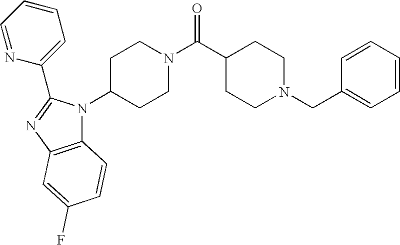 | 498 (ESMS) |
| 628 | 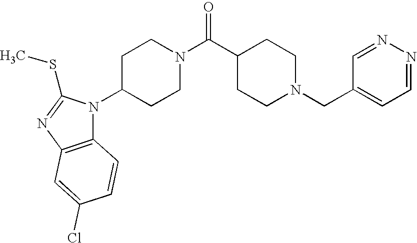 | 485 (ESMS) |
| 629 | 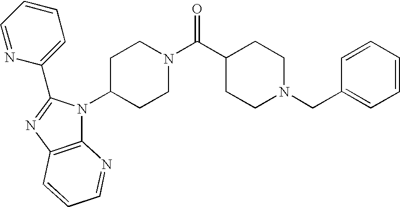 | 481 (ESMS) |
| 630 | 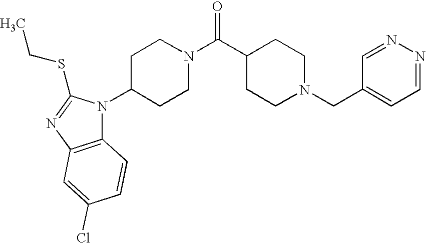 | 499 (ESMS) |
| 631 | 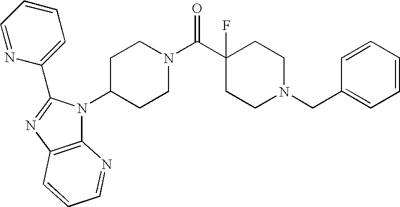 | 499 (ESMS) |
| 632 | 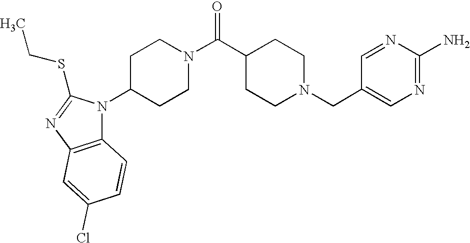 | 514 (ESMS) |
| 633 | 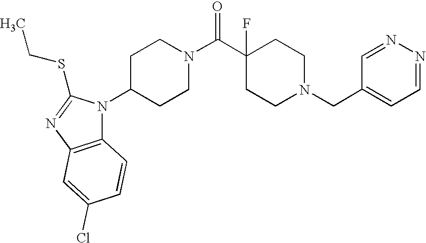 | 517 (ESMS) |
| 634 | 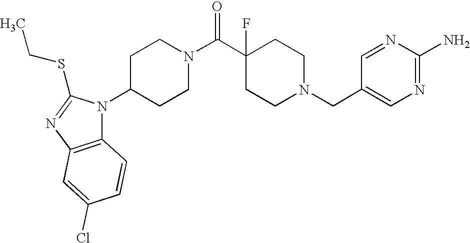 | 532 (ESMS) |
| 635 | 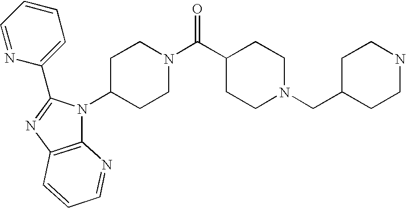 | 488 (ESMS) |
| 636 | 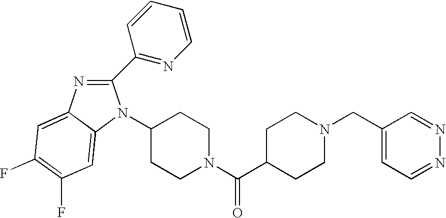 | 518 (ESMS) |
| 637 | 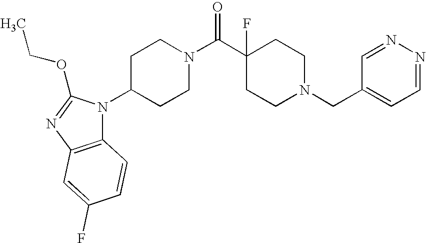 | 451 (ESMS) |
| 638 | 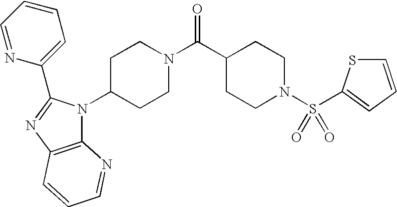 | 537 (MH+) |
| 639 | 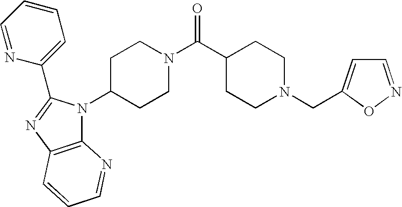 | 472 (MH+) |
| 640 | 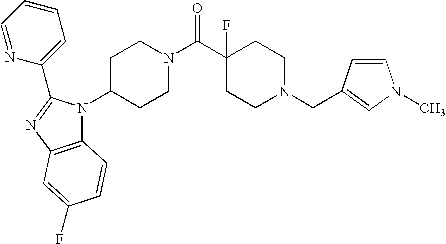 | 519 (MH+) |
| 641 | 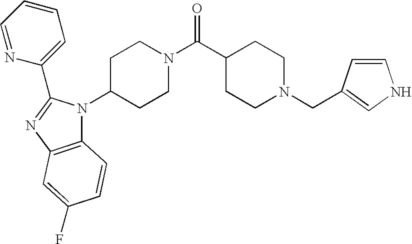 | 487 (MH+) |
| 642 | 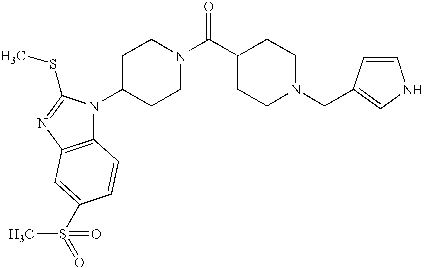 | 516 (MH+) |
| 643 | 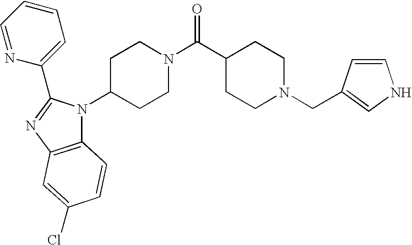 | 503 (MH+) |
| 644 | 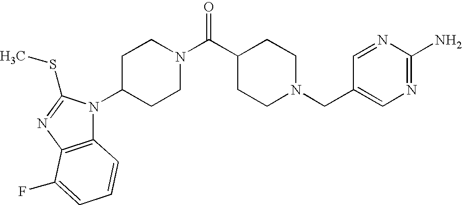 | 484 (ESMS) |
| 645 | 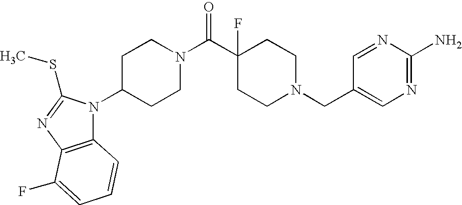 | 503 (ESMS) |
| 646 | 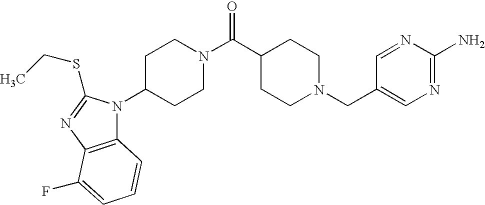 | 498 (ESMS) |
| 647 | 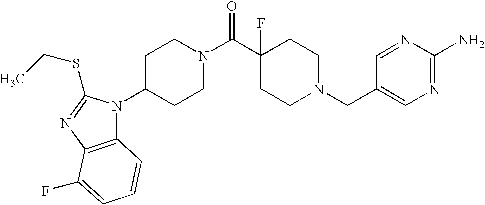 | 516 (ESMS) |
| 648 | 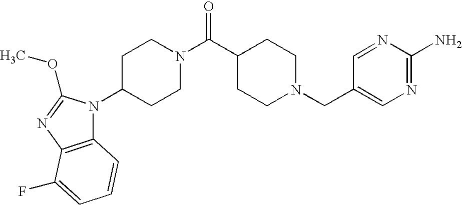 | 468 (ESMS) |
| 649 | 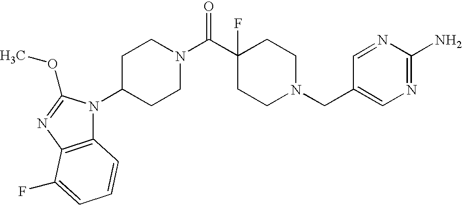 | 486 (ESMS) |
| 650 | 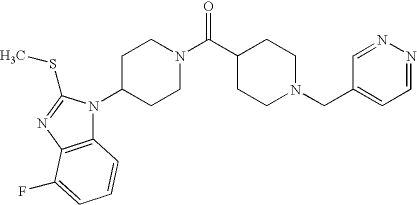 | 469 (ESMS) |
| 651 | 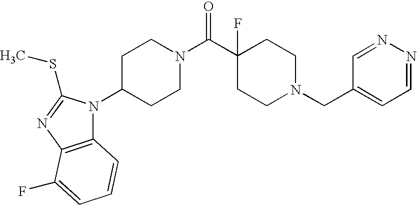 | 487 (ESMS) |
| 652 | 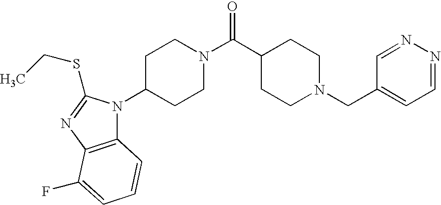 | 483 (ESMS) |
| 653 | 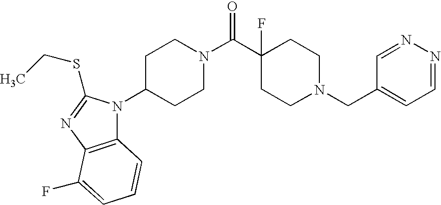 | 501 (ESMS) |
| 654 | 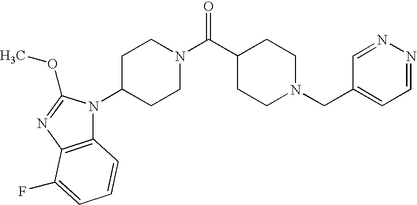 | 453 (ESMS) |
| 655 | 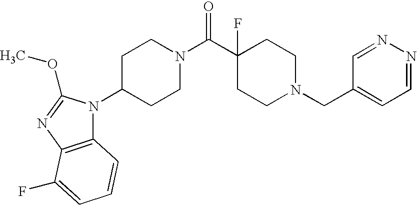 | 471 (ESMS) |
| 656 | 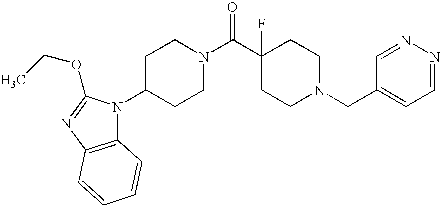 | 468 (ESMS) |
| 657 | 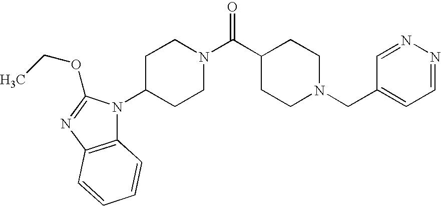 | 450 (ESMS) |
| 658 | 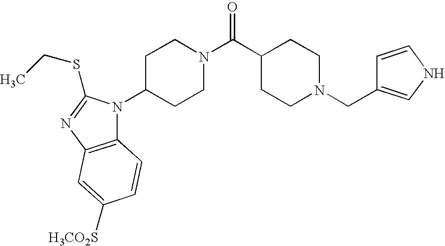 | 530 (ESMS) |
| 659 | 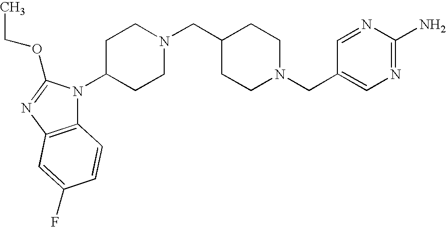 | 468 (FAB) |
| 660 | 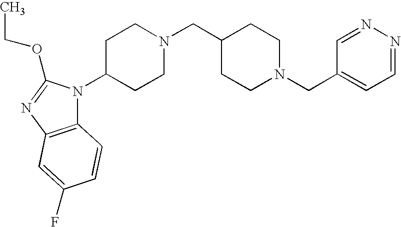 | 453 (FAB) |
| 661 | 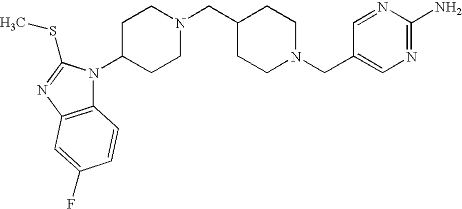 | 470 (FAB) |
| 662 | 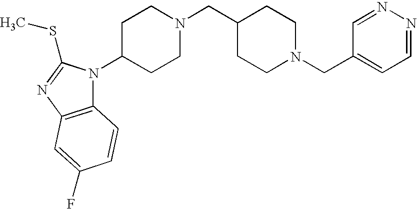 | 455 (FAB) |
| 663 | 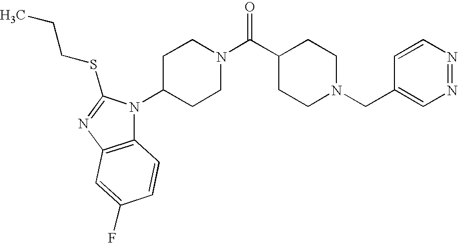 | 497 (ESMS) |
| 664 | 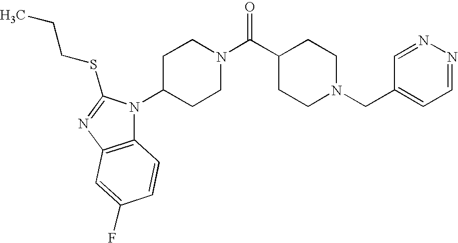 | 481 (FAB) |
| 664A | 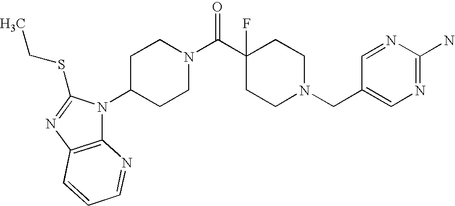 | 499 (FAB) |
| 665 | 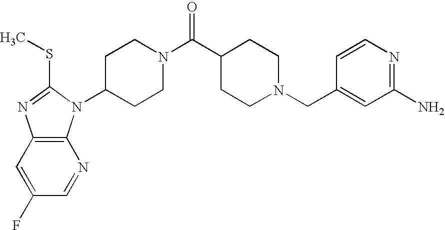 | NA |
| 666 | 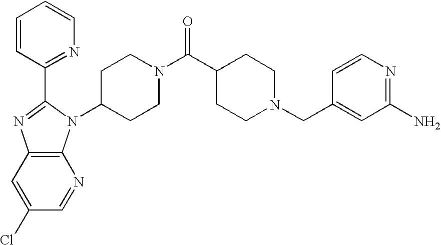 | NA |

NA = not available

### Example 20

### Preparation of Compound 185

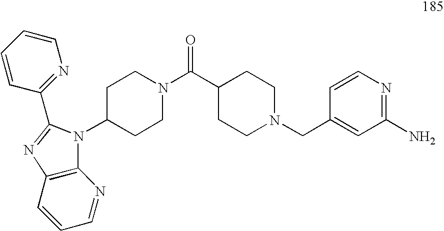

Step 1

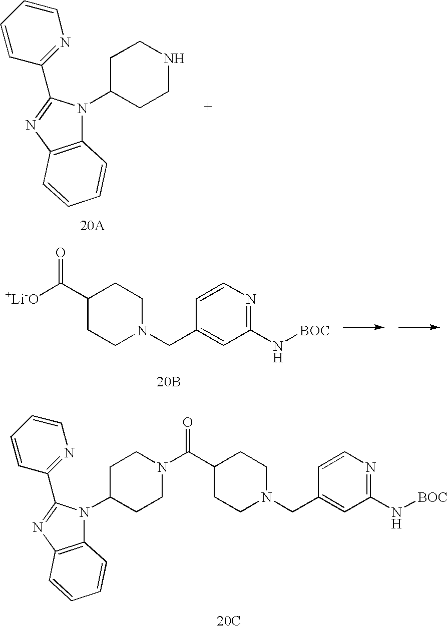

### Synthesis of Compounds 20A and 20B

Compounds 20A and 20B were prepared as described U.S. Pat. No. 7,105,505.

### Synthesis of Compound 20C

A mixture of 3.00 g (10.7 mmol) of compound 20A, 4.03 g (11.8 mmol) of compound 20B, 3.08 g (16.1 mmol) of EDC dihydrochloride, 2.18 g (16.1 mmol) of HOBT and 3.74 mL (26.8 mmol) of triethylamine was stirred in a mixture of 85 mL of CH₂Cl₂ and 25 mL of DMF at 60° C. for 5 hours. Solvent was removed in vacuo and the resulting residue was dissolved in CH₂Cl₂, then washed with aqueous NaHCO₃ and brine. The organic phase was separated, dried and concentrated in vacuo to provide a crude residue, which was then purified using flash column chromatography on silica gel (0.5-1.2% of 7M NH₃ in MeOH/CH₂Cl₂) to provide 6.29 g of compound 20C as a white solid.

Step 2

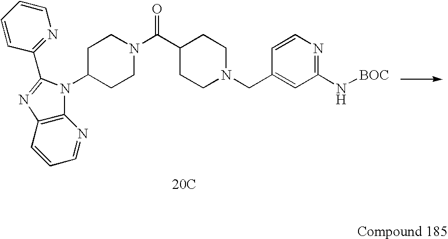

A solution of compound 20C (6.29 g) in 20% TFA/CH₂Cl₂ was allowed to stir at room temperature for about 15 hours. The reaction mixture was concentrated in vacuo and the reside obtained was subjected to basic aqueous work-up to provide 4.67 g of compound 185 as a white solid. MH⁺ 497

To 4.00 g (8.05 mmol) of the free base of compound 185 in a mixture of 50 mL of CH₂Cl₂ and 5 mL of MeOH, was added 4.05 mL of 2M HCl in ether solution (8.10 mmol). The solution was stirred at room temperature for 2 hours, then concentrated in vacuo to provide a residue that was dried in vacuo to provide 4.67 g of the hydrochloride salt of compound 185 as a white foam.

### Example 21

### Preparation of Compound 174

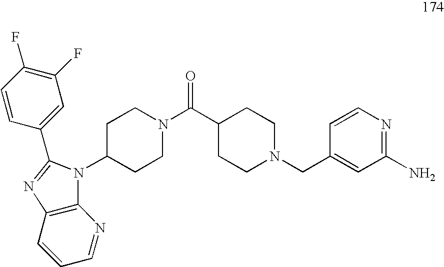

Step 1

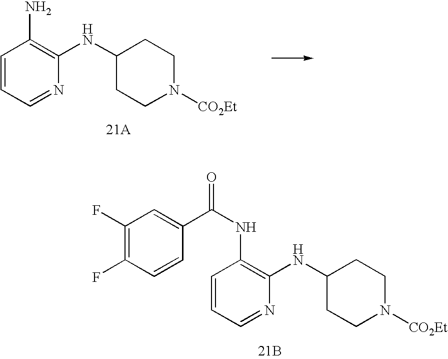

A solution of amine 21A (3.5 g; 0.32 mol, prepared as described in Example 5 of U.S. Pat. No. 7,105,505), 3,4-difluorobenzoic acid (55.0 g; 0.35 mol), EDC dihydrochloride (90.8 g; 0.47 mol), HOBT (64.0 g; 0.47 mol) and triethylamine (132 mL; 0.95 mol) was in a mixture of 1.5 L of DMF and 1.5 L of CH₂Cl₂ was heated to 70° C. and allowed to stir at this temperature for 21 hours, then cooled to room temperature and stirred for an additional 48 hours. The reaction mixture was then diluted with 6 L of ethyl acetate, 3 L of water and 0.5 L of brine and the organic phase was separated and was further washed with 2 L of water. The aqueous phase was back-extracted with 1 L of ethyl acetate and the combined organic extracts were washed with brine, dried over Na₂SO₄ and concentrated in vacuo to provide a crude purple oil, which was flash chromatographed on silica gel (1-5% MeOH/CH₂Cl₂) to provide 133.3 g of amide 21B as a brown oil.

Step 2

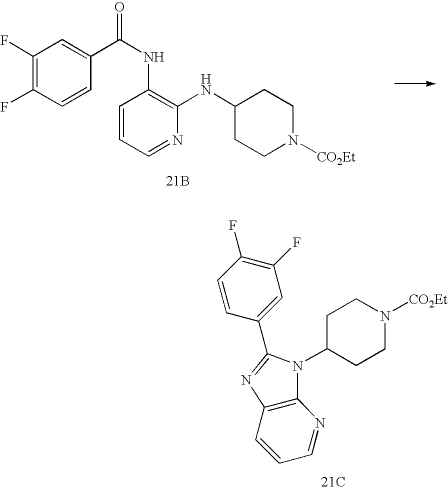

A solution of amide 21B (125 g; 0.3 mol) in 2.3 L of acetic acid was heated to 120° C. an allowed to stir at this temperature for about 12 hours. The reaction mixture was concentrated in vacuo and the resultant residue was partitioned between 1.5 L of saturated aqueous NaHCO₃ solution and 2.5 L of CH₂Cl₂. The organic phase was separated and the aqueous phase was extracted with CH₂Cl₂. The combined organic extracts were dried over Na₂SO₄, filtered and concentrated in vacuo to provide 104 g of a crude dark beige solid. The crude material was suspended in a mixture of 300 mL of ether and 100 mL of hexanes, stirred, filtered and dried at 50° C. for 0.5 h to provide 90 g of compound 21C as a beige solid.

Step 3

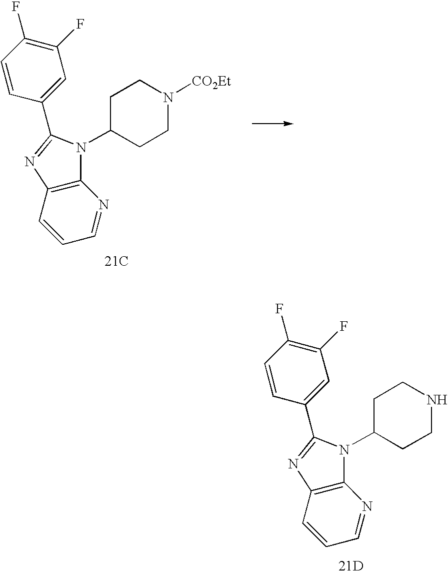

A mixture of carbamate 21C (90.0 g; 0.23 mol) and trimethylsilyl iodide (230 g; 1.17 mol) in 2.5 L of CHCl₃ was heated to 65° C. and allowed to stir at this temperature for 12 hours. The reaction mixture was then cooled to 10° C., and 1 L of 1M aqueous NaOH was added slowly, until the solution was at pH 13. 6 L of CH₂Cl₂ was added to the basified solution and the organic phase was separated, washed with water and brine, dried over Na₂SO₄, filtered and concentrated in vacuo to provide 56.5 g of amine 21D as a beige solid, which was used without further purification.

Step 4

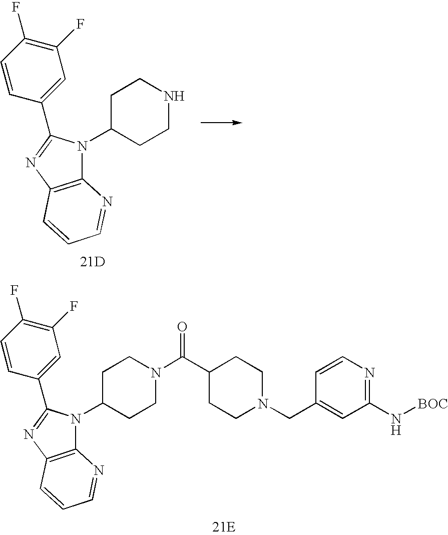

A solution of amine 21D (56.5 g; 0.18 mol), acid lithium salt 20B (73.7 g; 0.22 mol), EDC dihydrochloride (43.1 g; 0.23 mol), HOBT (30.4 g; 0.23 mol) and (i-Pr)₂NEt (62.9 mL; 0.36 mol) in 0.95 L of DMF and 0.95 L of CH₂Cl₂ was heated to 66° C. and allowed to stir at this temperature for 15 hours. The reaction mixture was then diluted with 6 L of ethyl acetate and washed with 3 L of water. The organic phase was collected and washed with 2 L of water, 1 L of brine, dried over Na₂SO₄, filtered and concentrated in vacuo to a volume of about 300 mL. The resulting suspension was filtered and the collected solid was washed with a mixture of CH₂Cl₂-ether-ethyl acetate, then diluted with CH₂Cl₂ (250 mL) and the resulting solution was concentrated in vacuo. The residue obtained was purified using flash column chromatography on silica gel (2-5% MeOH/CH₂Cl₂) to provide 30.0 g of compound 21E as a white foam. Additional impure compound 21E was also obtained in impure column fractions. The impure fractions were collected, concentrated down to 300 mL, and the resulting solution was diluted with 200 mL of ether. The white precipitate that resulted was filtered, washed with ethyl acetate and dried for about 15 hours to afford an additional 40 g of compound 21E.

Step 5

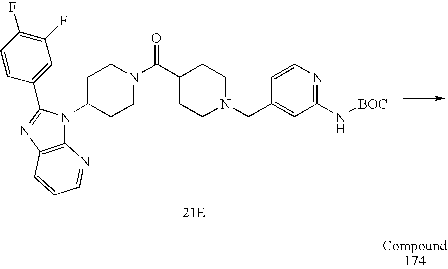

A solution of compound 21E (70.0 g) in 1.3 L of CH₂Cl₂ was cooled to about 8° C. and to the cooled solution was added dropwise 500 g of trifluoroacetic acid. The resulting reaction was allowed to warm to room temperature and stirred for 12 hours. The reaction mixture was then diluted with 2 L of water and the aqueous phase was collected. The organic phase was re-extracted twice, once with 1 L of water and then with 0.5 L of water. The combined aqueous extracts were combined and diluted with 4 L of CH₂Cl₂. To this mixture was added 2 L of aqueous NaOH solution (stock solution obtained by adding 240 mL of conc. NaOH solution to 3 L of water) and the resulting solution was at pH 10-11. The basified solution was then stirred at room temperature for 10 minutes. The organic phase was separated, dried over MgSO₄, filtered and concentrated in vacuo to provide a residue which was dried under vacuum to provide 59.8 g of a white glassy solid.

The white glassy solid was dissolved in 300 mL of CH₂Cl₂ and to the resulting solution was added 52 mL of 2M HCl in ether. The resulting mixture was allowed to stir at room temperature for 30 minutes, then the solution was diluted with 500 mL of ether, resulting in the formation of a white precipitate. Additional ether (500 mL) was added to the precipitate solution and the resulting mixture was allowed to stir at room temperature for 1 hour. The resulting precipitate was then filtered and the collected solid was dried in vacuo to provide 58.3 g of compound 174 as its hydrochloride salt. MH⁺ 532

### Example 22

### Preparation of Compound 666

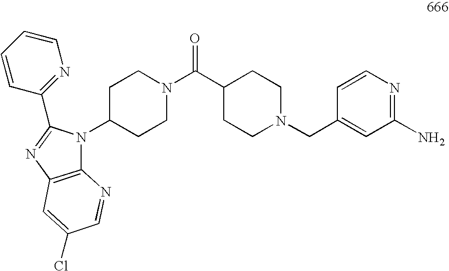

Step 1

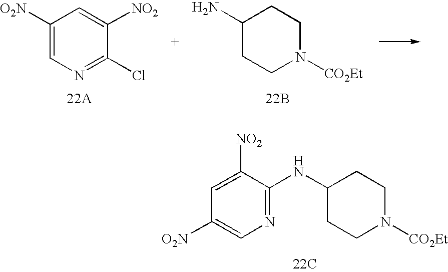

A mixture of 2-chloro-3,5-dinitropyridine 22A (9.63 g; 47.3 mmol), 4-aminopiperidine 22B (8.11 mL; 47.3 mmol) and triethylamine (8.55 mL; 61.5 mmol) in 200 mL of DMF was allowed to stir for about 15 hours at room temperature. The reaction mixture was concentrated in vacuo to provide a residue that was subsequently dissolved in a mixture of CH₂Cl₂-MeOH, then concentrated in vacuo, and the residue obtained was purified using flash column chromatography on silica gel (from 25% hexanes/CH₂Cl₂ to 0-2% acetone/CH₂Cl₂) to provide 15.8 g of dinitroamine 22C as a yellow solid.

Step 2

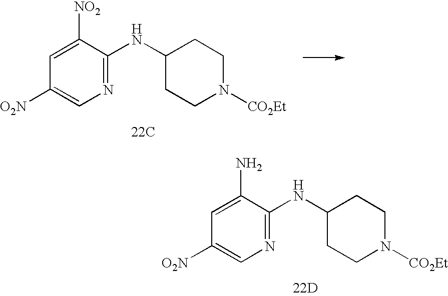

To a solution of dinitroamine 22C (15.4 g; 45.3 mmol) in 300 mL of EtOH was added 54 mL of 20% aqueous (NH₄)₂S solution (158.5 mmol), and the resulting reaction was allowed to stir at room temperature for 5 hours. The reaction mixture was concentrated in vacuo and the residue obtained was purified using flash column chromatography on silica gel (0-4% acetone/CH₂Cl₂) to provide 10.9 g of compound 22D as an orange solid.

Step 3

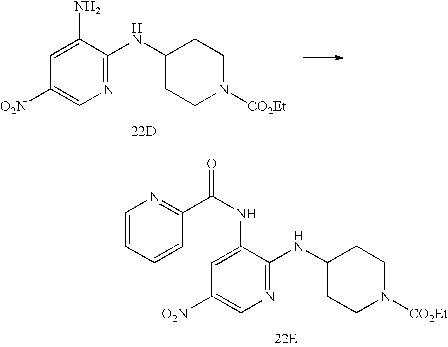

A mixture of 5.73 g (18.5 mmol) of amine 22D, pyridine-2-carboxylic acid (2.73 g; 22.2 mmol), EDC hydrochloride (5.31 g; 27.7 mmol), HOBT (3.75 g; 27.7 mmol) and triethylamine (5.00 mL; 35.9 mmol) in 100 mL of DMF and 100 mL of CH₂Cl₂ was heated to 65° C. and allowed to stir at this temperature for about 15 hours. The reaction mixture was concentrated in vacuo and the resulting residue was subjected to diluted with water, extracted with CH₂Cl₂ and the organic phase dried over MgSO₄, filtered and concentrated in vacuo. The residue obtained was purified using flash column chromatography on silica gel (0-0.6% MeOH/CH₂Cl₂) to provide 4.50 g of amide 22E as an orange foam.

Step 4

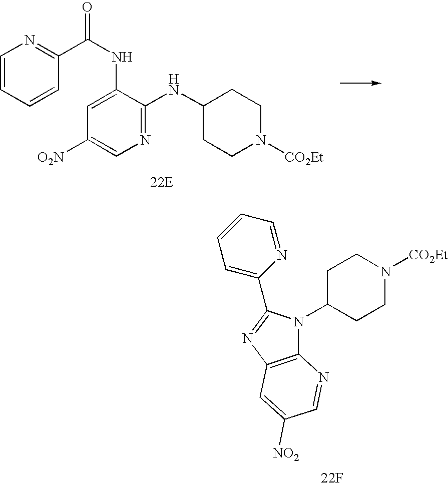

A mixture of amide 22E (4.50 g) in 90 mL of acetic acid was heated to 125° C. and allowed to stir at this temperature for about 15 hours. The reaction mixture was concentrated in vacuo and the resulting residue was partitioned between aqueous NaHCO₃ and CH₂Cl₂. The organic phase was separated, dried over MgSO₄, and concentrated in vacuo to provide benzimidazole 22F (3.88 g) as a beige foam which was used without further purification.

Step 5

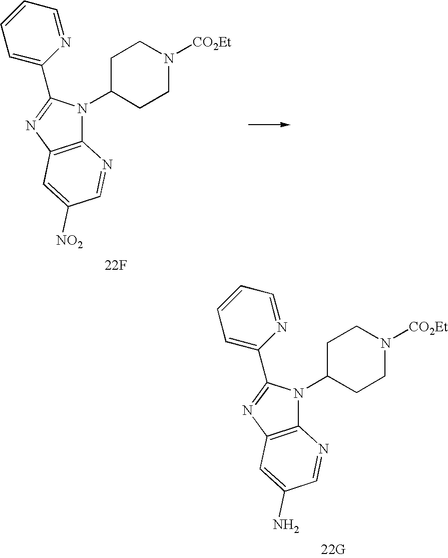

To a solution of nitrobenzimidazole 22F (3.88 g) in 125 mL of ethyl acetate was added 0.8 g of 10% Pd on activated carbon and the reaction was evacuated and put under hydrogen atmosphere using a hydrogen-filled balloon. The reaction mixture was allowed to stir under H₂ atmosphere for about 15 hours and was then filtered. The filtrate was concentrated in vacuo to provide 3.41 g of compound 22G as a yellow-tinted solid, which was used without further purification.

Step 6

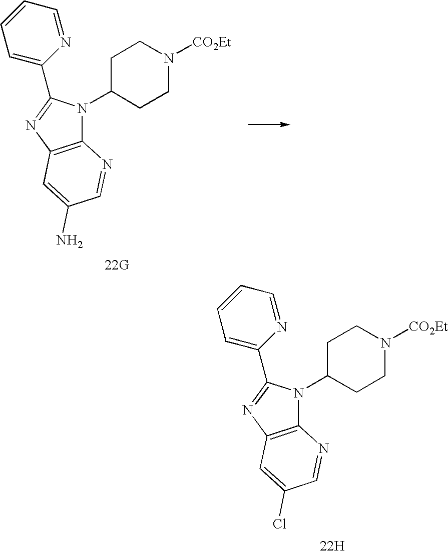

A solution of amine 22G (1.94 g; 5.29 mmol) in 50 mL of conc. HCl was cooled to 0° C. and to the cooled solution was slowly added an aqueous solution of NaNO₂ (0.47 g; 6.88 mmol). The resulting reaction was allowed to stir at 0° C. for 45 minutes, after which time CuCl (1.05 g; 10.6 mmol) was added portionwise. The resulting reaction was then allowed to warm to room temperature and allowed to stir at this temperature for 3 hours. The reaction mixture was neutralized to pH 7 using aqueous NaOH, which resulted in formation of a green precipitate. The resulting solution was diluted with CH₂Cl₂ and filtered through Celite. The organic layer was separated, dried over Na₂SO₄, filtered and concentrated in vacuo to provide a crude residue which was purified using flash column chromatography on silica gel (0.5-1.0% MeOH/CH₂Cl₂) to provide 1.49 g of chlorobenzimidazole 22H as a white solid.

Step 7

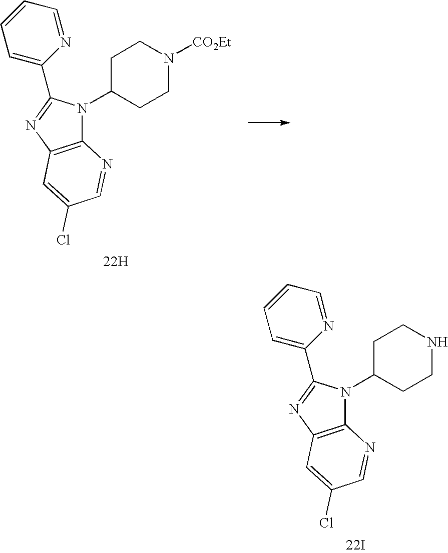

To a solution of carbamate 22H (1.49 g; 3.86 mmol) in 75 mL of CHCl₃ was added trimethylsilyl iodide (2.74 g; 19.3 mmol), and the resulting solution was heated to reflux and allowed to stir at this temperature for about 15 hours. The reaction mixture was cooled to room temperature, diluted with water, and the resulting solution was adjusted to pH 8 using 1M aqueous NaOH. The resulting solution was extracted with CH₂Cl₂, and the organic phase was separated, dried over Na₂SO₄, filtered and concentrated in vacuo. The crude residue obtained was purified using flash column chromatography on silica gel (1-2% 7M NH₃ in MeOH/CH₂Cl₂) to provide 725 mg of amine 221 as a white solid.

Steps 8-9

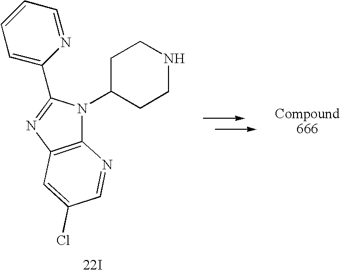

Compound 22I was converted into compound 666 using the methods described in steps 1 and 2 of Example 20. Compound 666 was obtained as a white solid free base. MH⁺ 531

### Example 23

### Guinea Pig H₃ Receptor Binding Assay

The source of the H₃ receptors in this experiment was guinea pig brain obtained from animals weighing 400-600 g. The brain tissue was homogenized with a solution of 50 mM Tris, pH 7.5. The final concentration of tissue in the homogenization buffer was 10% w/v. The homogenates were centrifuged at 1,000×g for 10 minutes in order to remove clumps of tissue and debris. The resulting supernatants were then centrifuged at 50,000×g for 20 minutes in order to sediment the membranes, which were then washed three times in homogenization buffer (50,000×g for 20 minutes each). The membranes were frozen and stored at −70° C. until needed.

All compounds to be tested were dissolved in DMSO and then diluted into the binding buffer (50 mM Tris, pH 7.5) such that the final concentration was 2 μg/mL with 0.1% DMSO. Membranes were then added (400 μg of protein) to the reaction tubes. The reaction was started by the addition of 3 nM [³H]R-α-methyl histamine (8.8 Ci/mmol) or 3 nM [³H]N^{α}-methyl histamine (80 Ci/mmol) and continued under incubation at 30° C. for 30 minutes. Bound ligand was separated from unbound ligand by filtration, and the amount of radioactive ligand bound to the membranes was quantitated by liquid scintillation spectrometry. All incubations were performed in duplicate and the standard error was always less than 10%. Compounds that inhibited more than 70% of the specific binding of radioactive ligand to the receptor were serially diluted to determine a Kᵢ (nM).

Compounds of formula I have a Kᵢ within the range of about 0.1 to about 600 nM. Preferred compounds of formula I have a Kᵢ within the range of about 0.1 to about 100 nM. More preferred compounds of formula I have a Kᵢ within the range of about 0.1 to about 20 nM.

### Example 24

### Human H₃ Receptor Binding Assay

The full-length human histamine H₃ receptor was cloned by PCR from a human thalamus cDNA library, with primers derived from a public database, and inserted into the CMV promoter-driven expression vector pcDNA-3.1 (Invitrogen). HEK-293 human embryonic kidney cells (ATCC) were transfected with H₃ receptor plasmid and stably expressing cells were selected with G-418. Cells were grown in Dulbecco's modified Eagle's medium/10% fetal calf serum containing high glucose, 25 mM Hepes, penicillin (100 U/ml), streptomycin (100 ug/ml), 2 mM glutamine, and 0.5 mg G-418/ml at 37° C. in a humidified atmosphere of 5% CO₂.

For membrane preparations, cells were harvested using aspirating media, replacing it with 5 mM EDTA/0.02% trypsin/Hank's balanced salt solution, followed by incubation at 37° C. for 5 to 10 minutes. Cells were decanted and centrifuged at 4° C. for 10 minutes at 1000×g, then resuspended in 50 mM Tris.HCl (ph 7.4) and disrupted for 30 seconds with a Polytron (PT10 tip at setting 6). Homogenates were then centrifuged for ten minutes at 1000×g and the supernatant was decanted and centrifuged for an additional ten minutes at 50,000×g. The pellets obtained were resuspended in Tris buffer and again centrifuged for ten minutes at 50,000×g. Membranes were stored at −80° C. as suspensions of 1 mg of protein/mL of Tris buffer.

For binding assays, membranes were dispersed by Polytron and incubated in 200 mL 50 mM Tris.HCl (pH 7.4) with 1 nM [3H]N-α-methylhistamine and a compound of the invention at concentrations, each in duplicate, equivalent to half orders of magnitude over a five order-of-magnitude range. Nonspecific binding was determined in the presence of 10-5 M thioperamide. After a 30 minute incubation at 30° C., assay mixtures were filtered through 0.3% polyethylenimine-soaked GF/B glass fiber filters, which were then rinsed thrice with buffer, dried, impregnated with Meltilex wax scintillant, and counted. IC₅₀ values were determined from curves fit to the data using a non-linear, least-squares, curve-fitting program and Ki values were determined using the method of Cheng and Prusoff.

### Example 25

### In Vivo Effect of Compound 174 on Glucose Levels in Diabetic Mice

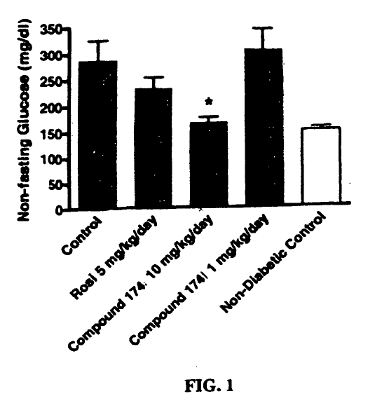

Five-week-old male ICR mice were purchased from Taconic Farm (Germantown, N.Y.) and placed on a “western diet” containing 45% (kcal) fat from lard and 0.12% (w/w) cholesterol. After 3 weeks of feeding, the mice were injected once with low dose streptozocin (STZ, ip 75-100 mg/kg) to induce partial insulin deficiency. Two weeks after receiving the STZ injection, the majority of the STZ-treated mice developed type 2 diabetes and displayed hyperglycemia, insulin resistance, and glucose intolerance. The diabetic mice were then placed in one of groups: (1) a non-treated diabetic control group, (2) a group treated with rosiglitazone (5 mg/kg/day in diet); (3) a group treated with Compound 174(10/mg/kg in diet) for four weeks; and (4) a group treated with Compound 174 (1/mg/kg in diet) for four weeks. Diabetic mice treated with Compound 174 (10 mg/kg/day in diet) had significantly reduced non-fasting glucose and HbA1C levels (see FIG. 1) relative to control mice and mice treated with rosiglitazone (5 mg/kg/day in diet).

Accordingly, Compound 174, an illustrative Compound of Formula (I) is effective for treating diabetes in a patient.

### Example 26

### In Vivo Effect of Compound 174 on HbA1c Levels in Diabetic Rats

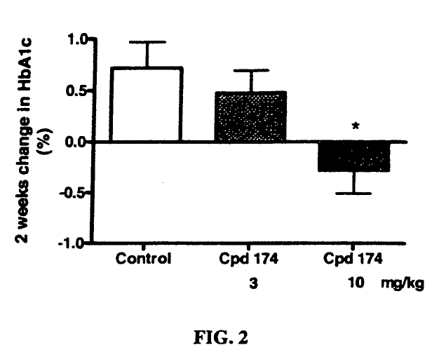

Seventy male DIO Sprague-Dawley rats were fed HFD (45% Kcal fat) for 3 months from weaning, and were given streptozotocin (STZ) intraperitoneally at 25 mg/kg to induce type 2 diabetes (T2DM). Forty four T2DM rats were chosen for the study two weeks after STZ injection (n=11 per group, with body weights between 632 and 838 g, non-fasting glucose between 226 and 426 mg/dl and HbA1c between 8.7% and 10.9%) and were given ad libitum access to pre-weighed 45% fat (kcal) HFD or Compound 287 (1.4, 2.9 mg/g in HFD) for two weeks. Body weight, non-fasting glucose and food intake were monitored daily. Body composition and HbA1c levels were monitored before and after the two-week study by the whole body magnetic resonance analyzer and Cholestech GDX analyzer (Hayward, Calif.), respectively. The STZ-DIO rats had elevated non-fasting glucose and HbA1c levels (non-fasting glucose were between 226 and 426 mg/dl; and HbA1c were between 8.7% and 10.9%) two weeks after STZ injection. The low dose of STZ caused a 48% reduction of plasma insulin levels, which was not sufficient to cause hyperglycemia in rats fed with chow diet. In contrast, this level of plasma insulin induced hyperglycemia in the face of insulin resistance induced by the HFD. As illustrated in FIG. 2, Compound 287 caused a dose-dependent reduction of non-fasting blood glucose and HbA1c levels over the two week study period. The control STZ-DIO rats maintained non-fasting glucose levels above 350 mg/ml (+12 mg/dl), which led to a significant 0.96% increase in HbA1c over 14 days. STZ-DIO rats treated with Compound 287 (68 mg/kg/day, 2.9 mg/g in HFD) had significantly reduced non-fasting glucose (−43 mg/dl) which led to a 0.6% decrease in HbA1c level in two weeks (see FIG. 2).

Accordingly, Compound 287, an illustrative Compound of Formula (I) is effective for treating diabetes in a patient.

### Methods of Using the Compounds of Formula (I)

The Compounds of Formula (I) are useful for treating or preventing a Condition a patient.

### Methods for Treating or Preventing Pain

The Compounds of Formula (I) are useful for treating or preventing pain in a patient.

Accordingly, in one embodiment, the present invention provides a method for treating pain in a patient, comprising administering to the patient an effective amount of one or more Compounds of Formula (I).

Illustrative examples of pain treatable or preventable using the present methods, include, but are not limited to acute pain, chronic pain, neuropathic pain, nociceptive pain, cutaneous pain, somatic pain, visceral pain, phantom limb pain, diabetic pain, cancer pain (including breakthrough pain), pain caused by drug therapy (such as cancer chemotherapy), headache (including migraine, tension headache, cluster headache, pain caused by arithritis, pain caused by injury, toothache, or pain caused by a medical procedure (such as surgery, physical therapy or radiation therapy).

In one embodiment, the pain is neuropathic pain.

In another embodiment, the pain is cancer pain.

In another embodiment, the pain is headache.

In still another embodiment, the pain is chronic pain.

In a further embodiment, the pain is diabetic pain.

### Methods for Treating or Preventing Diabetes

The Compounds of Formula (I) are useful for treating or preventing diabetes in a patient. Accordingly, in one embodiment, the present invention provides a method for treating diabetes in a patient, comprising administering to the patient an effective amount of one or more Compounds of Formula (I).

Examples of diabetes treatable or preventable using the Compounds of Formula (I) include, but are not limited to, type I diabetes (insulin-dependent diabetes mellitus), type II diabetes (non-insulin dependent diabetes mellitus), gestational diabetes, diabetes caused by administration of anti-psychotic agents, diabetes caused by administration of anti-depressant agents, diabetes caused by administration of steroid drugs, autoimmune diabetes, insulinopathies, diabetes due to pancreatic disease, diabetes associated with other endocrine diseases (such as Cushing's Syndrome, acromegaly, pheochromocytoma, glucagonoma, primary aldosteronism or somatostatinoma), type A insulin resistance syndrome, type B insulin resistance syndrome, lipatrophic diabetes, diabetes induced by β-cell toxins, and diabetes induced by drug therapy (such as diabetes induced by antipsychotic agents).

In one embodiment, the diabetes is type I diabetes.

In another embodiment, the diabetes is type II diabetes.

In another embodiment, the diabetes is gestational diabetes.

### Methods for Treating or Preventing a Diabetic Complication

The Compounds of Formula (I) are useful for treating or preventing a diabetic complication in a patient. Accordingly, in one embodiment, the present invention provides a method for treating a diabetic complication in a patient, comprising administering to the patient an effective amount of one or more Compounds of Formula (I).

Examples of diabetic complications treatable or preventable using the Compounds of Formula (I) include, but are not limited to, diabetic cataract, glaucoma, retinopathy, aneuropathy (such as diabetic neuropathy, polyneuropathy, mononeuropathy, autonomic neuropathy, microaluminuria and progressive diabetic neuropathyl), nephropathy, diabetic pain, gangrene of the feet, immune-complex vasculitis, systemic lupsus erythematosus (SLE), atherosclerotic coronary arterial disease, peripheral arterial disease, nonketotic hyperglycemic-hyperosmolar coma, foot ulcers, joint problems, a skin or mucous membrane complication (such as an infection, a shin spot, a candidal infection or necrobiosis lipoidica diabeticorumobesity), hyperlipidemia, hypertension, syndrome of insulin resistance, coronary artery disease, a fungal infection, a bacterial infection, and cardiomyopathy.

In one embodiment, the diabetic complication is neuropathy.

In another embodiment, the diabetic complication is retinopathy.

In another embodiment, the diabetic complication is nephropathy.

### Methods for Treating or Preventing Impaired Glucose Tolerance

The Compounds of Formula (I) are useful for treating or preventing impaired glucose tolerance in a patient.

Accordingly, in one embodiment, the present invention provides a method for treating impaired glucose tolerance in a patient, comprising administering to the patient an effective amount of one or more Compounds of Formula (I).

### Methods for Treating or Preventing Impaired Fasting Glucose

The Compounds of Formula (I) are useful for treating or preventing impaired fasting glucose in a patient.

Accordingly, in one embodiment, the present invention provides a method for treating impaired fasting glucose in a patient, comprising administering to the patient an effective amount of one or more Compounds of Formula (I).

### Combination Therapy

Accordingly, in one embodiment, the present invention provides methods for treating a Condition in a patient, the method comprising administering to the patient one or more Compounds of Formula (I), or a pharmaceutically acceptable salt, solvate, ester or prodrug thereof and at least one additional therapeutic agent that is not a Compound of Formula (I), wherein the amounts administered are together effective to treat or prevent a Condition.

When administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. The amounts of the various actives in such combination therapy may be different amounts (different dosage amounts) or same amounts (same dosage amounts).

In one embodiment, the one or more Compounds of Formula (I) is administered during at time when the additional therapeutic agent(s) exert their prophylactic or therapeutic effect, or vice versa.

In another embodiment, the one or more Compounds of Formula (I) and the additional therapeutic agent(s) are administered in doses commonly employed when such agents are used as monotherapy for treating a Condition.

In another embodiment, the one or more Compounds of Formula (I) and the additional therapeutic agent(s) are administered in doses lower than the doses commonly employed when such agents are used as monotherapy for treating a Condition.

In still another embodiment, the one or more Compounds of Formula (I) and the additional therapeutic agent(s) act synergistically and are administered in doses lower than the doses commonly employed when such agents are used as monotherapy for treating a Condition.

In one embodiment, the one or more Compounds of Formula (I) and the additional therapeutic agent(s) are present in the same composition. In one embodiment, this composition is suitable for oral administration. In another embodiment, this composition is suitable for intravenous administration.

The one or more Compounds of Formula (I) and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

In one embodiment, the administration of one or more Compounds of Formula (I) and the additional therapeutic agent(s) may inhibit the resistance of a Condition to these agents.

In one embodiment, when the patient is treated for diabetes, a diabetic complication, impaired glucose tolerance or impaired fasting glucose, the other therapeutic is an antidiabetic agent which is not a Compound of Formula (I). In another embodiment, when the patient is treated for pain, the other therapeutic agent is an analgesic agent which is not a Compound of Formula (I).

In another embodiment, the other therapeutic agent is an agent useful for reducing any potential side effect of a Compound of Formula (I). Such potential side effects include, but are not limited to, nausea, vomiting, headache, fever, lethargy, muscle aches, diarrhea, general pain, and pain at an injection site.

In one embodiment, the other therapeutic agent is used at its known therapeutically effective dose. In another embodiment, the other therapeutic agent is used at its normally prescribed dosage. In another embodiment, the other therapeutic agent is used at less than its normally prescribed dosage or its known therapeutically effective dose.

Examples of antidiabetic agents useful in the present methods for treating diabetes or a diabetic complication include a sulfonylurea; an insulin sensitizer (such as a PPAR agonist, a DPP-IV inhibitor, a PTP-1B inhibitor and a glucokinase activator); a glucosidase inhibitor; an insulin secretagogue; a hepatic glucose output lowering agent;an anti-obesity agent; an antihypertensive agent; a meglitinide; an agent that slows or blocks the breakdown of starches and sugars in vivo; an histamine H₃ receptor antagonist; an antihypertensive agent, a sodium glucose uptake transporter 2 (SGLT-2) inhibitor; a peptide that increases insulin production; and insulin or any insulin-containing composition.

In one embodiment, the antidiabetic agent is an insulin sensitizer or a sulfonylurea.

Non-limiting examples of sulfonylureas include glipizide, tolbutamide, glyburide, glimepiride, chlorpropamide, acetohexamide, gliamilide, gliclazide, glibenclamide and tolazamide.

Non-limiting examples of insulin sensitizers include PPAR activators, such as troglitazone, rosiglitazone, pioglitazone and englitazone; biguanidines such as metformin and phenformin; DPP-IV inhibitors; PTP-1B inhibitors; and α-glucokinase activators, such as miglitol, acarbose, and voglibose.

Non-limiting examples of DPP-IV inhibitors useful in the present methods include sitagliptin, saxagliptin (Januvia™, Merck), denagliptin, vildagliptin (Galvus™, Novartis), alogliptin, alogliptin benzoate, ABT-279 and ABT-341 (Abbott), ALS-2-0426 (Alantos), ARI-2243 (Arisaph), BI-A and BI-B (Boehringer Ingelheim), SYR-322 (Takeda), MP-513 (Mitsubishi), DP-893 (Pfizer), RO-0730699 (Roche) or a combination of sitagliptin/metformin HCl (Janumet™, Merck).

Non-limiting examples of SGLT-2 inhibitors useful in the present methods include dapagliflozin and sergliflozin, AVE2268 (Sanofi-Aventis) and T-1095 (Tanabe Seiyaku).

Non-limiting examples of hepatic glucose output lowering agents include Glucophage and Glucophage XR.

Non-limiting examples of histamine H₃ receptor antagonist agents include the following compound:

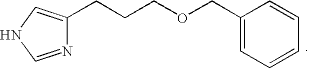

Non-limiting examples of insulin secretagogues include sulfonylurea and non-sulfonylurea drugs such as GLP-1, a GLP-1 mimetic, exendin, GIP, secretin, glipizide, chlorpropamide, nateglinide, meglitinide, glibenclamide, repaglinide and glimepiride.

Non-limiting examples of GLP-1 mimetics useful in the present methods include Byetta-Exanatide, Liraglutinide, CJC-1131 (ConjuChem, Exanatide-LAR (Amylin), BIM-51077 (Ipsen/LaRoche), ZP-10 (Zealand Pharmaceuticals), and compounds disclosed in International Publication No. WO 00/07617.

The term “insulin” as used herein, includes all formulations of insulin, including long acting and short acting forms of insulin.

Non-limiting examples of orally administrable insulin and insulin containing compositions include AL-401 from AutoImmune, and the compositions disclosed in U.S. Pat. Nos. 4,579,730; 4,849,405; 4,963,526; 5,642,868; 5,763,396; 5,824,638; 5,843,866; 6,153,632; 6,191,105; and International Publication No. WO 85/05029, each of which is incorporated herein by reference.

In one embodiment, the antidiabetic agent is anti-obesity agent.

Non-limiting examples of anti-obesity agents useful in the present methods for treating diabetes include a 5-HT2C agonist, such as lorcaserin; a neuropeptide Y antagonist; an MCR4 agonist; an MCH receptor antagonist; a protein hormone, such as leptin or adiponectin; an AMP kinase activator; and a lipase inhibitor, such as orlistat. Appetite suppressants are not considered to be within the scope of the anti-obesity agents useful in the present methods.

Non-limiting examples of antihypertensive agents useful in the present methods for treating diabetes include β-blockers and calcium channel blockers (for example diltiazem, verapamil, nifedipine, amlopidine, and mybefradil), ACE inhibitors (for example captopril, lisinopril, enalapril, spirapril, ceranopril, zefenopril, fosinopril, cilazopril, and quinapril), AT-1 receptor antagonists (for example losartan, irbesartan, and valsartan), renin inhibitors and endothelin receptor antagonists (for example sitaxsentan).

Non-limiting examples of meglitinides useful in the present methods for treating diabetes include repaglinide and nateglinide.

Non-limiting examples of insulin sensitizing agents include biguanides, such as metformin, metformin hydrochloride (such as GLUCOPHAGE® from Bristol-Myers Squibb), metformin hydrochloride with glyburide (such as GLUCOVANCE™ from Bristol-Myers Squibb) and buformin; glitazones; and thiazolidinediones, such as rosiglitazone, rosiglitazone maleate (AVANDIA™ from GlaxoSmithKline), pioglitazone, pioglitazone hydrochloride (ACTOS™, from Takeda) ciglitazone and MCC-555 (Mitstubishi Chemical Co.)

In one embodiment, the insulin sensitizer is a thiazolidinedione.

In another embodiment, the insulin sensitizer is a biguanide.

In another embodiment, the insulin sensitizer is a DPP-IV inhibitor.

In a further embodiment, the antidiabetic agent is a SGLT-2 inhibitor.

Non-limiting examples of antidiabetic agents that slow or block the breakdown of starches and sugars and are suitable for use in the compositions and methods of the present invention include alpha-glucosidase inhibitors and certain peptides for increasing insulin production. Alpha-glucosidase inhibitors help the body to lower blood sugar by delaying the digestion of ingested carbohydrates, thereby resulting in a smaller rise in blood glucose concentration following meals. Non-limiting examples of suitable alpha-glucosidase inhibitors include acarbose; miglitol; camiglibose; certain polyamines as disclosed in WO 01/47528 (incorporated herein by reference); voglibose. Non-limiting examples of suitable peptides for increasing insulin production including amlintide (CAS Reg. No. 122384-88-7 from Amylin; pramlintide, exendin, certain compounds having Glucagon-like peptide-1 (GLP-1) agonistic activity as disclosed in WO 00/07617 (incorporated herein by reference).

Non-limiting examples of orally administrable insulin and insulin containing compositions include AL-401 from AutoImmune, and the compositions disclosed in U.S. Pat. Nos. 4,579,730; 4,849,405; 4,963,526; 5,642,868; 5,763,396; 5,824,638; 5,843,866; 6,153,632; 6,191,105; and International Publication No. WO 85/05029, each of which is incorporated herein by reference.

Non-limiting examples of other analgesic agents useful in the present methods for treating pain include acetaminophen, an NSAID, an opiate or a tricyclic antidepressant.

In one embodiment, the other analgesic agent is acetaminophen or an NSAID.

In another embodiment, the other analgesic agent is an opiate.

In another embodiment, the other analgesic agent is a tricyclic antidepressant.

Non-limiting examples of NSAIDS useful in the present methods for treating pain include a salicylate, such as aspirin, amoxiprin, benorilate or diflunisal; an arylalkanoic acid, such as diclofenac, etodolac, indometacin, ketorolac, nabumetone, sulindac or tolmetin; a 2-arylpropionic acid (a “profen”), such as ibuprofen, carprofen, fenoprofen, flurbiprofen, loxoprofen, naproxen, tiaprofenic acid or suprofen; a fenamic acid, such as mefenamic acid or meclofenamic acid; a pyrazolidine derivative, such as phenylbutazone, azapropazone, metamizole or oxyphenbutazone; a coxib, such as celecoxib, etoricoxib, lumiracoxib or parecoxib; an oxicam, such as piroxicam, lornoxicam, meloxicam or tenoxicam; or a sulfonanilide, such as nimesulide.

Non-limiting examples of opiates useful in the present methods for treating pain include an anilidopiperidine, a phenylpiperidine, a diphenylpropylamine derivative, a benzomorphane derivative, an oripavine derivative and a morphinane derivative. Additional illustrative examples of opiates include morphine, diamorphine, heroin, buprenorphine, dipipanone, pethidine, dextromoramide, alfentanil, fentanyl, remifentanil, methadone, codeine, dihydrocodeine, tramadol, pentazocine, vicodin, oxycodone, hydrocodone, percocet, percodan, norco, dilaudid, darvocet or lorcet.

Non-limiting examples of tricyclic antidepressants useful in the present methods for treating pain include amitryptyline, carbamazepine, gabapentin or pregabalin.

The doses and dosage regimen of the other agents used in the combination therapies of the present invention for the treatment or prevention of a Condition can be determined by the attending clinician, taking into consideration the the approved doses and dosage regimen in the package insert; the age, sex and general health of the patient; and the type and severity of the viral infection or related disease or disorder. When administered in combination, the Compound(s) of Formula (I) and the other agent(s) for treating diseases or conditions listed above can be administered simultaneously or sequentially. This is particularly useful when the components of the combination are given on different dosing schedules, e.g., one component is administered once daily and another every six hours, or when the preferred pharmaceutical compositions are different, e.g. one is a tablet and one is a capsule. A kit comprising the separate dosage forms is therefore advantageous.

Generally, a total daily dosage of the one or more Compounds of Formula (I) and the additional therapeutic agent(s) can when administered as combination therapy, range from about 0.1 to about 2000 mg per day, although variations will necessarily occur depending on the target of the therapy, the patient and the route of administration. In one embodiment, the dosage is from about 0.2 to about 100 mg/day, administered in a single dose or in 2-4 divided doses. In another embodiment, the dosage is from about 1 to about 500 mg/day, administered in a single dose or in 2-4 divided doses. In another embodiment, the dosage is from about 1 to about 200 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In a further embodiment, the dosage is from about 1 to about 20 mg/day, administered in a single dose or in 2-4 divided doses.

### Compositions and Administration

In one embodiment, the invention provides compositions comprising an effective amount of one or more Compounds of Formula (I) or a pharmaceutically acceptable salt, solvate, ester or prodrug thereof, and a pharmaceutically acceptable carrier.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

In one embodiment, the Compound of Formula (I) is administered orally.

In another embodiment, the Compound of Formula (I) is administered parenterally.

In another embodiment, the Compound of Formula (I) is administered intravenously.

In one embodiment, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation is from about 0.1 to about 2000 mg. Variations will necessarily occur depending on the target of the therapy, the patient and the route of administration. In one embodiment, the unit dose dosage is from about 0.2 to about 1000 mg. In another embodiment, the unit dose dosage is from about 1 to about 500 mg. In another embodiment, the unit dose dosage is from about 1 to about 100 mg/day. In still another embodiment, the unit dose dosage is from about 1 to about 50 mg. In yet another embodiment, the unit dose dosage is from about 1 to about 10 mg.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 300 mg/day, preferably 1 mg/day to 75 mg/day, in two to four divided doses.

When the invention comprises a combination of at least one Compound of Formula (I) and an additional therapeutic agent, the two active components may be co-administered simultaneously or sequentially, or a single pharmaceutical composition comprising at least one Compound of Formula (I) and an additional therapeutic agent in a pharmaceutically acceptable carrier can be administered. The components of the combination can be administered individually or together in any conventional dosage form such as capsule, tablet, powder, cachet, suspension, solution, suppository, nasal spray, etc. The dosage of the additional therapeutic agent can be determined from published material, and may range from about 1 to about 1000 mg per dose. In one embodiment, when used in combination, the dosage levels of the individual components are lower than the recommended individual dosages because of the advantageous effect of the combination.

In one embodiment, the components of a combination therapy regime are to be administered simultaneously, they can be administered in a single composition with a pharmaceutically acceptable carrier.

In another embodiment, when the components of a combination therapy regime are to be administered separately or sequentially, they can be administered in separate compositions, each containing a pharmaceutically acceptable carrier.

The components of the combination therapy can be administered individually or together in any conventional dosage form such as capsule, tablet, powder, cachet, suspension, solution, suppository, nasal spray, etc.

### Kits

In one aspect, the present invention provides a kit comprising a effective amount of one or more Compounds of Formula (I), or a pharmaceutically acceptable salt or solvate of the compound and a pharmaceutically acceptable carrier, vehicle or diluent.

In another aspect the present invention provides a kit comprising an amount of one or more Compounds of Formula (I), or a pharmaceutically acceptable salt or solvate of the compound and an amount of at least one additional therapeutic agent listed above, wherein the combined amounts are effective for treating or preventing a Condition in a patient.

When the components of a combination therapy regime are to are to be administered in more than one composition, they can be provided in a kit comprising in a single package, one container comprising a Compound of Formula (I) in pharmaceutically acceptable carrier, and one or more separate containers, each comprising one or more additional therapeutic agents in a pharmaceutically acceptable carrier, with the active components of each composition being present in amounts such that the combination is therapeutically effective.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited herein, the entire disclosures of which are incorporated herein by reference.

What is claimed is:

1. A method for treating a condition in a patient, comprising administering to the patient an effective amount of one or more compounds having the formula: 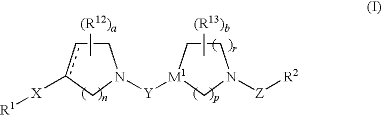
or a pharmaceutically acceptable salt, solvate, ester or prodrug thereof, wherein:
the dotted line represents an optional and additional bond;
M¹ is C(R³);
X is a bond or C₁-C₆ alkylene;
Y is —C(O)—, —C(S)—, —(CH₂)_{q}—, —C(O)NR⁴—, —C(O)CH₂—, —SO₂—, or —C(═N—CN)—NH—, such that when M¹ is N, Y is not —C(O)NR⁴— or —C(═N—CN)—NH—.
Z is a bond, C₁-C₆ alkylene, C₁-C₆ alkenylene, —C(O)—, —CH(CN)—, or —CH₂C(O)NR⁴—;
R¹ is 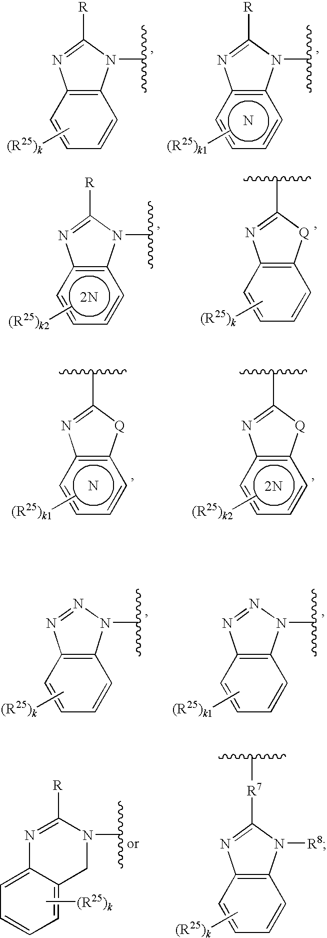
Q is —N(R⁸)—, —S— or —O—;
R is H, OH, C₁-C₆ alkyl, halo(C₁-C₆)alkyl-, C₁-C₆ alkoxy, (C₁-C₆)alkoxy-(C₁-C₆)alkyl-, (C₁-C₆)-alkoxy-(C₁-C₆)alkoxy, (C₁-C₆)alkoxy-(C₁-C₆)alkyl-SO₀₋₂, R³²-aryl(C₁-C₆)alkoxy-, R³²-aryl(C₁-C₆)alkyl-, R³²-aryl, R³²-aryloxy, R³²-heteroaryl, (C₃-C₆)cycloalkyl, (C₃-C₆)cyclo-alkyl-(C₁-C₆)alkyl, (C₃-C₆)cycloalkyl-(C₁-C₆)alkoxy, (C₃-C₆)cycloalkyl-oxy-, R³⁷-hetero-cycloalkyl, N(R³⁰)(R³¹)—(C₁-C₆)alkyl-, —N(R³⁰)(R³¹), —NH—(C₁-C₆)alkyl-O—(C₁-C₆)alkyl, —NHC(O)NH(R²⁹); R²⁹—S(O)₀₋₂—, halo(C₁-C₆)alkyl-S(O)₀₋₂—, N(R³⁰)(R³¹)—(C₁-C₆)alkyl-S(O)₀₋₂— or benzoyl;
R² is a six-membered heteroaryl ring having 1 or 2 heteroatoms independently selected from N or N—O, with the remaining ring atoms being carbon; a five-membered heteroaryl ring having 1, 2 or 3 heteroatoms independently selected from N, O or S, with the remaining ring atoms being carbon; R³²-quinolyl; R³²-aryl; heterocycloalkyl; 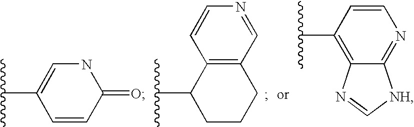
wherein said six-membered heteroaryl ring or said five-membered heteroaryl ring is optionally substituted by R⁶;
R³ is H, halo, C₁-C₆ alkyl, —OH or (C₁-C₆)alkoxy;
R⁴ is independently selected from the group consisting of hydrogen, C₁-C₆ alkyl, C₃-C₆ cycloalkyl, (C₃-C₆)cycloalkyl(C₁-C₆)alkyl, R³³-aryl, R³³-aryl(C₁-C₆)alkyl, and R³²-heteroaryl;
R⁵ is hydrogen, C₁-C₆ alkyl, —C(O)R²⁰, —C(O)₂R²⁰, —C(O)N(R²⁰)₂, (C₁-C₆)alkyl-SO₂—, or (C₁-C₆)alkyl-SO₂—NH—;
R⁶ is 1 to 3 substituents independently selected from the group consisting of —OH, halo, C₁-C₆ alkyl-, C₁-C₆ alkoxy, C₁-C₆ alkylthio, —CF₃, —NR⁴R⁵, phenyl, R³³-phenyl, NO₂, —CO₂R⁴, —CON(R⁴)₂, 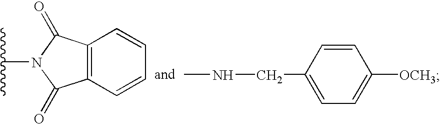
R⁷ is —N(R²⁹)—, —O— or —SO₀₋₂—;
R⁸ is H, C₁-C₆ alkyl, halo(C₁-C₆)alkyl-, (C₁-C₆)alkoxy-(C₁-C₆)alkyl-, R³²-aryl(C₁-C₆)alkyl-, R³²-aryl, R³²-heteroaryl, (C₃-C₆)cycloalkyl, (C₃-C₆)cycloalkyl-(C₁-C₆)alkyl, R³⁷-heterocycloalkyl, N(R³⁰)(R³¹)—(C₁-C₆)alkyl-, R²⁹—S(O)₂—,halo(C₁-C₆)alkyl-S(O)₂—, R²⁹—S(O)₀₋₁—(C₂-C₆)alkyl-, halo(C₁-C₆)alkyl-S(O)₀₋₁—(C₂-C₆)alkyl-;
R¹² is independently selected from the group consisting of C₁-C₆ alkyl, hydroxyl, C₁-C₆ alkoxy, or fluoro, provided that when R¹² is hydroxy or fluoro, then R¹² is not bound to a carbon adjacent to a nitrogen; or R¹² forms a C₁ to C₂ alkyl bridge from one ring carbon to another ring carbon;
R¹³ is independently selected from the group consisting of C₁-C₆ alkyl, hydroxyl, C₁-C₆ alkoxy, or fluoro, provided that when R¹³ is hydroxy or fluoro then R¹³ is not bound to a carbon adjacent to a nitrogen; or forms a C₁ to C₂ alkyl bridge from one ring carbon to another ring carbon; or R¹³ is ═O;
R²⁰ is independently selected from the group consisting of hydrogen, C₁-C₆ alkyl, or aryl, wherein said aryl group is optionally substituted with from 1 to 3 groups independently selected from halo, —CF₃, —OCF₃, hydroxyl, or methoxy; or when two R²⁰ groups are present, said two R²⁰ groups taken together with the nitrogen to which they are bound form a five or six membered heterocyclic ring;
R²² is C₁-C₆ alkyl, R³⁴-aryl or heterocycloalkyl;
R²⁴ is H, C₁-C₆ alkyl, —SO₂R²² or R³⁴-aryl;
R²⁵ is independently selected from the group consisting of C₁-C₆ alkyl, halo, —CF₃, —OH, C₁-C₆ alkoxy, (C₁-C₆)alkyl-C(O)—, aryl-C(O)—, N(R⁴)(R⁵)—C(O)—, N(R⁴)(R⁵)—S(O)₁₋₂—, halo-(C₁-C₆)alkyl- or halo-(C₁-C₆)alkoxy-(C₁-C₆)alkyl-;
R²⁹ is H, C₁-C₆ alkyl, C₃-C₆ cycloalkyl, R³⁵-aryl or R³⁵-aryl(C₁-C₆)alkyl-;
R³⁰ is H, C₁-C₆ alkyl-, R³⁵-aryl or R³⁵-aryl(C₁-C₆)alkyl-;
R³¹ is H, C₁-C₆ alkyl-, R³⁵-aryl, R³⁵-aryl(C₁-C₆)alkyl-, R³⁵-heteroaryl, (C₁-C₆)alkyl-C(O)—, R³⁵-aryl-C(O)—, N(R⁴)(R⁵)—C(O)—, (C₁-C₆)alkyl-S(O)₂— or R³⁵-aryl-S(O)₂—;
or R³⁰ and R³¹ together are —(CH₂)₄₋₅—, —(CH₂)₂—O—(CH₂)₂— or —(CH₂)₂—N(R³⁸)—(CH₂)₂— and form a ring with the nitrogen to which they are attached;
R³² is 1 to 3 substituents independently selected from the group consisting of H, —OH, halo, C₁-C₆ alkyl, C₁-C₆ alkoxy, R³⁵-aryl-O—, —SR²², —CF₃, —OCF₃, —OCHF₂, —NR⁴R⁵, phenyl, R³³-phenyl, NO₂, —CO₂R⁴, —CON(R⁴)₂, —S(O)₂R²², —S(O)₂N(R²⁰)₂, —N(R²⁴)S(O)₂R²², —CN, hydroxy-(C₁-C₆)alkyl-, —OCH₂CH₂OR²², and R³⁵-aryl(C₁-C₆)alkyl-O—, or two R³² groups on adjacent carbon atoms together form a —OCH₂O— or —O(CH₂)₂O— group;
R³³ is 1 to 3 substituents independently selected from the group consisting of C₁-C₆ alkyl, halo, —CN, —NO₂, —CF₃, —OCF₃, —OCHF₂ and —O—(C₁-C₆)alkyl;
R³⁴ is 1 to 3 substituents independently selected from the group consisting of H, halo, —CF₃, —OCF₃, —OH and —OCH₃.
R³⁵ is 1 to 3 substituents independently selected from hydrogen, halo, C₁-C₆ alkyl, hydroxy, C₁-C₆ alkoxy, phenoxy, —CF₃, —N(R³⁶)₂, —COOR²⁰ and —NO₂;
R³⁶ is independently selected form the group consisting of H and C₁-C₆ alkyl;
R³⁷ is 1 to 3 substituents independently selected from hydrogen, halo, C₁-C₆ alkyl, hydroxy, C₁-C₆ alkoxy, phenoxy, —CF₃, —N(R³⁶)₂, —COOR²⁰, —C(O)N(R²⁹)₂ and —NO₂, or R³⁷ is one or two ═O groups;
R³⁸ is H, C₁-C₆ alkyl, R³⁵-aryl, R³⁵-aryl(C₁-C₆)alkyl-, (C₁-C₆)alkyl-SO₂ or halo(C₁-C₆)alkyl-SO₂—;
a is 0, 1 or 2;
b is 0, 1 or 2;
k is 0, 1, 2, 3 or 4;
k1 is 0, 1, 2 or 3;
k2 is 0, 1 or 2;
n is 2;
p is 1, 2 or 3;
q is an integer ranging from 1 to 5; and
r is an integer ranging from 0 to 3,
such that: (i) when M² is N, p is not 1; (ii) when r is 0, M² is C; and (iii) the sum of p and r is 3,
wherein the condition is diabetes, a diabetic complication, impaired glucose tolerance or impaired fasting glucose.

2. The method of claim 1, wherein R¹ is

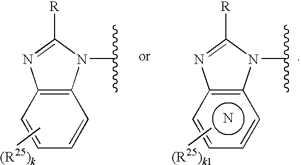

3. The method of claim 2, wherein R is alkoxy, alkoxyalkoxy, alkylthio, heteroaryl or R³²-aryl.

4. The method of claim 3, wherein R is —OCH₃, —OCH₂CH₃, —OCH((CH₃)₂, —SCH₃, —SCH₂CH₃, pyridyl (especially 2-pyridyl), pyrimidyl, pyrazinyl, furanyl, oxazolyl or R³²-phenyl.

5. The method of claim 2, wherein R¹ is:

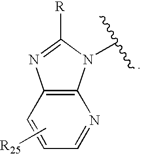

6. The method of claim 5, wherein R¹ is:

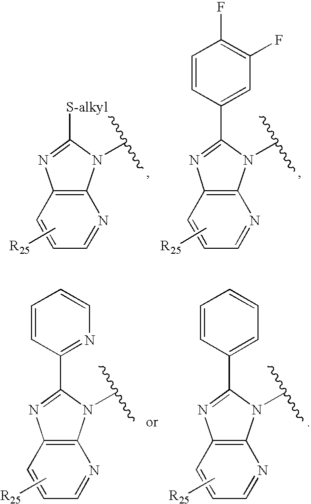

7. The method of claim 6, wherein R¹ is:

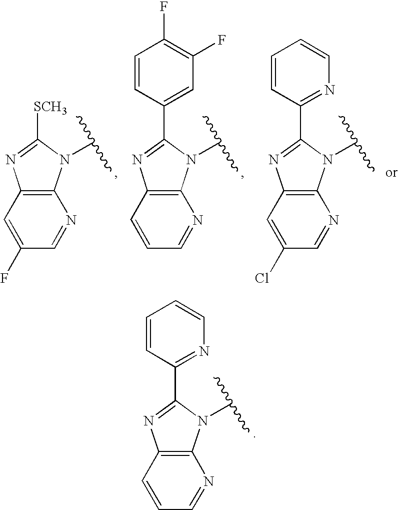

8, The method of claim 1, wherein R² is a six-membered heteroaryl group.

9. The method of claim 8, wherein R² is optionally substituted pyrimidyl or pyridyl.

10. The method of claim 9, wherein R² is

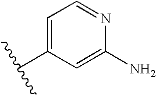

11. The method of claim 1, wherein X is a bond.

12. The method of claim 1, wherein Y is —C(O)—.

13. The method of claim 1, wherein Z is C₁-C₆ alkylene.

14. The method of claim 1, wherein Z is —CH₂—.

15. The method of claim 1, wherein M¹ is CH.

16. The method of claim 1, wherein M¹ is CF.

17. The method of claim 15, wherein n is 2, p is 2 and r is 1.

18. The method of claim 12, wherein M¹ is CH.

19. The method of claim 18, wherein n is 2, p is 2 and r is 1.

20. The method of claim 19, wherein a and b are each 0.

21. The method of claim 11, wherein R¹ is optionally substituted benzimidazolyl or 4-azabenzimidazolyl; and R² is a six-membered heteroaryl.

22. The method of claim 21, wherein Z is —CH₂— and R² is pyridyl or pyrimidyl.

23. The method of claim 22, wherein R¹ is

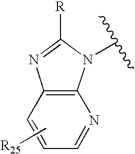

24. The method of claim 23, wherein R¹ is

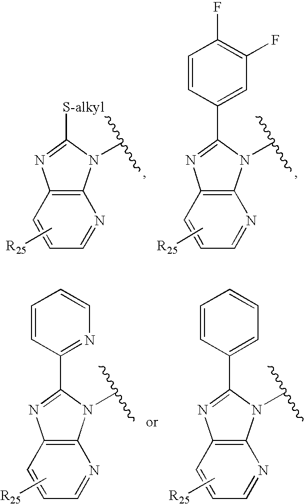

25. The method of claim 24, wherein R¹ is 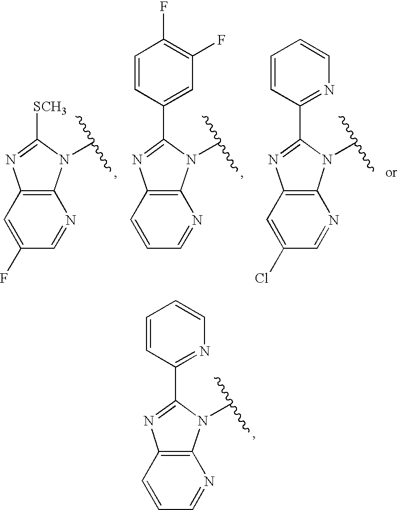
and R² is

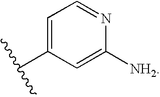

26. The method of claim 1, wherein the one or more compounds of formula (I) are selected from: 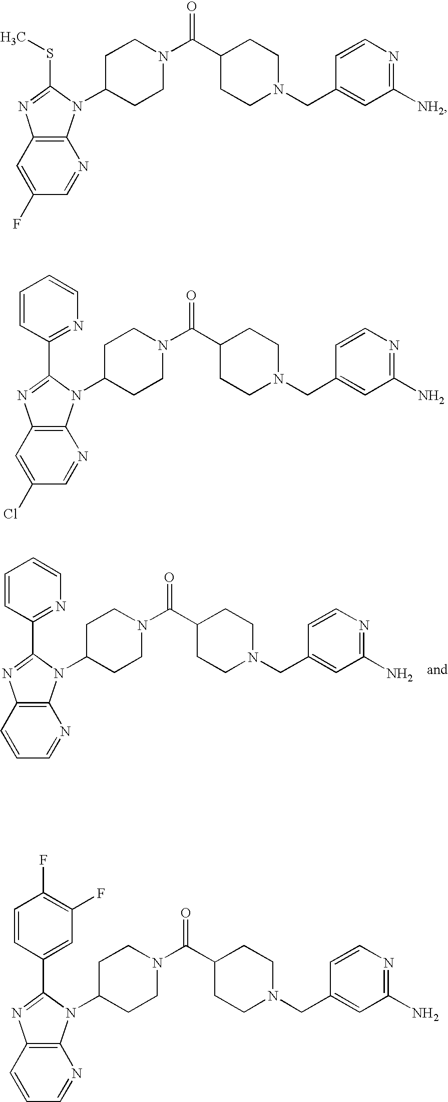
or a pharmaceutically acceptable salt, solvate, ester or prodrug thereof.

27. The method of claim 1, further comprising administering to the patient an additional antidiabetic agent that is not a compound of formula (I), wherein the amounts of the compound of Formula (I) and the additional antidiabetic agent are together effective to treat diabetes.

28. The method of claim 28, wherein the additional antidiabetic agent is selected from a sulfonylurea, an insulin sensitizer, an α-glucosidase inhibitor, an insulin secretagogue, an antiobesity agent, a meglitinide, insulin or an insulin-containing composition.

29. The method of claim 29, wherein the additional antidiabetic agent is an insulin sensitizer or a sulfonylurea.

30. The method of claim 30, wherein the insulin sensitizer is a PPAR activator.

31. The method of claim 29, wherein the additional antidiabetic agent is an antiobesity agent.

32. The method of claim 32, wherein the antiobesity agent is selected from: a neuropeptide Y antagonist, an MCR4 agonist, an MCH receptor antagonist, a protein hormone, an AMP kinase activator, and a lipase inhibitor.

33. The method of claim 33, wherein antiobesity agent is orlistat, leptin, or adiponectin.

34. The method of claim 1, wherein the condition treated is diabetes.

35. The method of claim 35, wherein the diabetes is type I diabetes.

36. The method of claim 35, wherein the diabetes is type II diabetes.

37. The method of claim 34, wherein the one or more compounds of formula (I) are selected from: 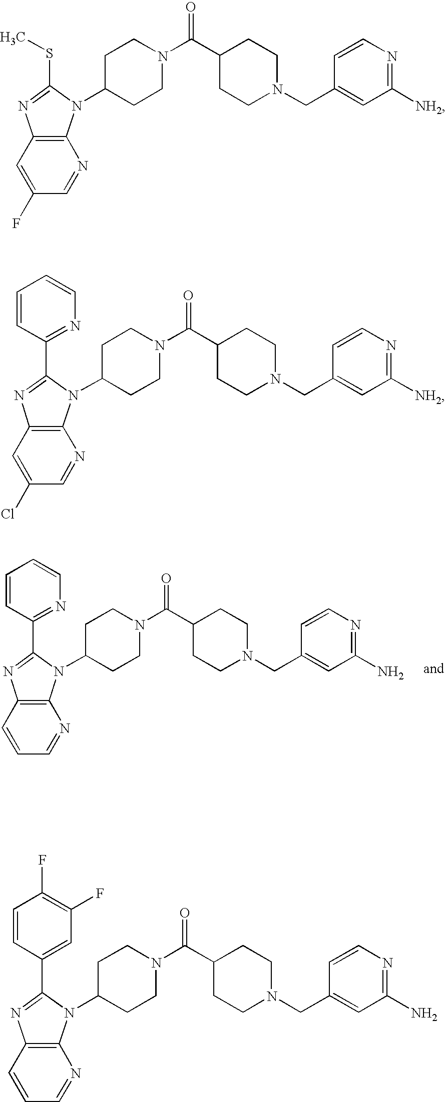
or a pharmaceutically acceptable salt, solvate, ester or prodrug thereof.

38. The method of claim 1, wherein the condition treated is a diabetic complication.

39. The method of claim 38, wherein the diabetic complication is diabetic cataract, glaucoma, retinopathy, neuropathy, nephropathy, gangrene of the feet, immune-complex vasculitis, systemic lupsus erythematosus, atherosclerotic coronary arterial disease, peripheral arterial disease, nonketotic hyperglycemic-hyperosmolar coma, foot ulcers or joint problems.

40. The method of claim 39, wherein the diabetic complication is neuropathy.

41. The method of claim 39, wherein the diabetic complication is retinopathy.

42. The method of claim 39, wherein the diabetic complication is nephropathy.

43. The method of claim 1, wherein the condition treated is impaired glucose tolerance.

44. The method of claim 1, wherein the condition treated is impaired fasting glucose.

45. A method for treating pain in a patient, comprising administering to the patient an effective amount of one or more compounds having the formula: 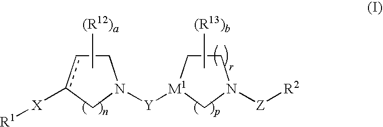
or a pharmaceutically acceptable salt, solvate, ester or prodrug thereof, wherein:
the dotted line represents an optional and additional bond;
M¹ is C(R³);
X is a bond or C₁-C₆ alkylene;
Y is —C(O)—, —C(S)—, —(CH₂)_{q}—, —C(O)NR⁴—, —C(O)CH₂—, —SO₂—, or —C(═N—CN)—NH—, such that when M¹ is N, Y is not —C(O)NR⁴— or —C(═N—CN)—NH—.
Z is a bond, C₁-C₆ alkylene, C₁-C₆ alkenylene, —C(O)—, —CH(CN)—, or —CH₂C(O)NR⁴—;
R¹ is 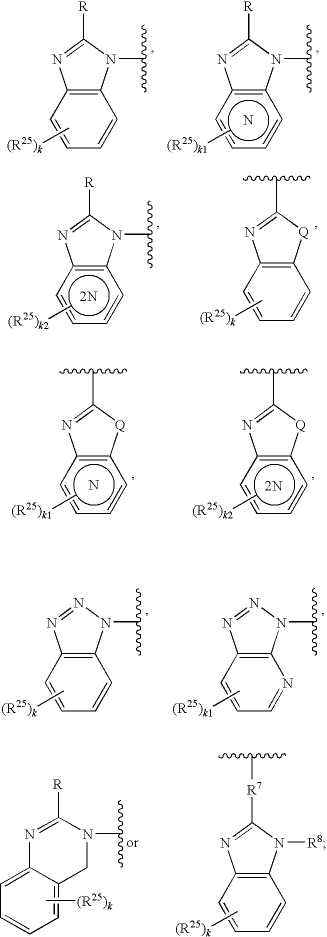
Q is —N(R⁸)—, —S— or —O—;
R is H, OH, C₁-C₆ alkyl, halo(C₁-C₆)alkyl-, C₁-C₆ alkoxy, (C₁-C₆)alkoxy-(C₁-C₆)alkyl-, (C₁-C₆)-alkoxy-(C₁-C₆)alkoxy, (C₁-C₆)alkoxy-(C₁-C₆)alkyl-SO₀₋₂, R³²-aryl(C₁-C₆)alkoxy-, R³²-aryl(C₁-C₆)alkyl-, R³²-aryl, R³²-aryloxy, R³²-heteroaryl, (C₃-C₆)cycloalkyl, (C₃-C₆)cyclo-alkyl-(C₁-C₆)alkyl, (C₃-C₆)cycloalkyl-(C₁-C₆)alkoxy, (C₃-C₆)cycloalkyl-oxy-, R³⁷-hetero-cycloalkyl, N(R³⁰)(R³¹)—(C₁-C₆)alkyl-, —N(R³⁰)(R³¹), —NH—(C₁-C₆)alkyl-O—(C₁-C₆)alkyl, —NHC(O)NH(R²⁹); R²⁹—S(O)₀₋₂—, halo(C₁-C₆)alkyl-S(O)₀₋₂—, N(R³⁰)(R³¹)—(C₁-C₆)alkyl-S(O)₀₋₂— or benzoyl;
R² is a six-membered heteroaryl ring having 1 or 2 heteroatoms independently selected from N or N—O, with the remaining ring atoms being carbon; a five-membered heteroaryl ring having 1, 2 or 3 heteroatoms independently selected from N, O or S, with the remaining ring atoms being carbon; R³²-quinolyl; R³²-aryl; heterocycloalkyl; 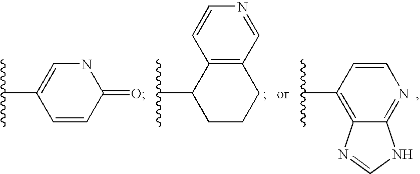
wherein said six-membered heteroaryl ring or said five-membered heteroaryl ring is optionally substituted by R⁶;
R³ is H, halo, C₁-C₆ alkyl, —OH or (C₁-C₆)alkoxy;
R⁴ is independently selected from the group consisting of hydrogen, C₁-C₆ alkyl, C₃-C₆ cycloalkyl, (C₃-C₆)cycloalkyl(C₁-C₆)alkyl, R³³-aryl, R³³-aryl(C₁-C₆)alkyl, and R³²-heteroaryl;
R⁵ is hydrogen, C₁-C₆ alkyl, —C(O)R²⁰, —C(O)₂R²⁰, —C(O)N(R²⁰)₂, (C₁-C₆)alkyl-SO₂—, or (C₁-C₆)alkyl-SO₂—NH—;
R⁶ is 1 to 3 substituents independently selected from the group consisting of —OH, halo, C₁-C₆ alkyl-, C₁-C₆ alkoxy, C₁-C₆ alkylthio, —CF₃, —NR⁴R⁵, phenyl, R³³-phenyl, NO₂, —CO₂R⁴, —CON(R⁴)₂, 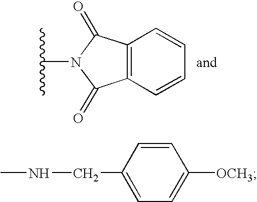
R⁷ is —N(R²⁹)—, —O— or —SO₀₋₂—;
R⁸ is H, C₁-C₆ alkyl, halo(C₁-C₆)alkyl-, (C₁-C₆)alkoxy-(C₁-C₆)alkyl-, R³²-aryl(C₁-C₆)alkyl-, R³²-aryl, R³²-heteroaryl, (C₃-C₆)cycloalkyl, (C₃-C₆)cycloalkyl-(C₁-C₆)alkyl, R³⁷-heterocycloalkyl, N(R³⁰)(R³¹)—(C₁-C₆)alkyl-, R²⁹—S(O)₂—, halo(C₁-C₆)alkyl-S(O)₂—, R²⁹—S(O)₀₋₁—(C₂-C₆)alkyl-, halo(C₁-C₆)alkyl-S(O)₀₋₁—(C₂-C₆)alkyl-;
R¹² is independently selected from the group consisting of C₁-C₆ alkyl, hydroxyl, C₁-C₆ alkoxy, or fluoro, provided that when R¹² is hydroxy or fluoro, then R¹² is not bound to a carbon adjacent to a nitrogen; or R¹² forms a C₁ to C₂ alkyl bridge from one ring carbon to another ring carbon;
R¹³ is independently selected from the group consisting of C₁-C₆ alkyl, hydroxyl, C₁-C₆ alkoxy, or fluoro, provided that when R¹³ is hydroxy or fluoro then R¹³ is not bound to a carbon adjacent to a nitrogen; or forms a C₁ to C₂ alkyl bridge from one ring carbon to another ring carbon; or R¹³ is ═O;
R²⁰ is independently selected from the group consisting of hydrogen, C₁-C₆ alkyl, or aryl, wherein said aryl group is optionally substituted with from 1 to 3 groups independently selected from halo, —CF₃, —OCF₃, hydroxyl, or methoxy; or when two R²⁰ groups are present, said two R²⁰ groups taken together with the nitrogen to which they are bound form a five or six membered heterocyclic ring;
R²² is C₁-C₆ alkyl, R³⁴-aryl or heterocycloalkyl;
R²⁴ is H, C₁-C₆ alkyl, —SO₂R²² or R³⁴-aryl;
R²⁵ is independently selected from the group consisting of C₁-C₆ alkyl, halo, —CF₃, —OH, C₁-C₆ alkoxy, (C₁-C₆)alkyl-C(O)—, aryl-C(O)—, N(R⁴)(R⁵)—C(O)—, N(R⁴)(R⁵)—S(O)₁₋₂—, halo-(C₁-C₆)alkyl- or halo-(C₁-C₆)alkoxy-(C₁-C₆)alkyl-;
R²⁹ is H, C₁-C₆ alkyl, C₃-C₆ cycloalkyl, R³⁵-aryl or R³⁵-aryl(C₁-C₆)alkyl-;
R³⁰ is H, C₁-C₆ alkyl-, R³⁵-aryl or R³⁵-aryl(C₁-C₆)alkyl-;
R³¹ is H, C₁-C₆ alkyl-, R³⁵-aryl, R³⁵-aryl(C₁-C₆)alkyl-, R³⁵-heteroaryl, (C₁-C₆)alkyl-C(O)—, R³⁵-aryl-C(O)—, N(R⁴)(R⁵)—C(O)—, (C₁-C₆)alkyl-S(O)₂— or R³⁵-aryl-S(O)₂—;
or R³⁰ and R³¹ together are —(CH₂)₄₋₅—, —(CH₂)₂—O—(CH₂)₂— or —(CH₂)₂—N(R³⁸)—(CH₂)₂— and form a ring with the nitrogen to which they are attached;
R³² is 1 to 3 substituents independently selected from the group consisting of H, —OH, halo, C₁-C₆ alkyl, C₁-C₆ alkoxy, R³⁵-aryl-O—, —SR²², —CF₃, —OCF₃, —OCHF₂, —NR⁴R⁵, phenyl, R³³-phenyl, NO₂, —CO₂R⁴, —CON(R⁴)₂, —S(O)₂R²², —S(O)₂N(R²⁰)₂, —N(R²⁴)S(O)₂R²², —CN, hydroxy-(C₁-C₆)alkyl-, —OCH₂CH₂OR²², and R³⁵-aryl(C₁-C₆)alkyl-O—, or two R³² groups on adjacent carbon atoms together form a —OCH₂O— or —O(CH₂)₂O— group;
R³³ is 1 to 3 substituents independently selected from the group consisting of C₁-C₆ alkyl, halo, —CN, —NO₂, —CF₃, —OCF₃, —OCHF₂ and —O—(C₁-C₆)alkyl;
R³⁴ is 1 to 3 substituents independently selected from the group consisting of H, halo, —CF₃, —OCF₃, —OH and —OCH₃.
R³⁵ is 1 to 3 substituents independently selected from hydrogen, halo, C₁-C₆ alkyl, hydroxy, C₁-C₆ alkoxy, phenoxy, —CF₃, —N(R³⁶)₂, —COOR²⁰ and —NO₂;
R³⁶ is independently selected form the group consisting of H and C₁-C₆ alkyl;
R³⁷ is 1 to 3 substituents independently selected from hydrogen, halo, C₁-C₆ alkyl, hydroxy, C₁-C₆ alkoxy, phenoxy, —CF₃, —N(R³⁶)₂, —COOR²⁰, —C(O)N(R²⁹)₂ and —NO₂, or R³⁷ is one or two ═O groups;
R³⁸ is H, C₁-C₆ alkyl, R³⁵-aryl, R³⁵-aryl(C₁-C₆)alkyl-, (C₁-C₆)alkyl-SO₂ or halo(C₁-C₆)alkyl-SO₂—;
a is 0, 1 or 2;
b is 0, 1 or 2;
k is 0, 1, 2, 3 or 4;
k1 is 0, 1, 2 or 3;
k2 is 0, 1 or 2;
n is 2;
p is 1, 2 or 3;
q is an integer ranging from 1 to 5; and
r is an integer ranging from 0 to 3,
such that: (i) when M² is N, p is not 1; (ii) when r is 0, M² is C; and (iii) the sum of p and r is 3.

46. The method of claim 45, wherein the compound of formula (I) is a compound of claim 26 or a pharmaceutically acceptable salt, solvate, ester or prodrug thereof.

47. The method of claim 46, wherein the compound of formula (I) is: 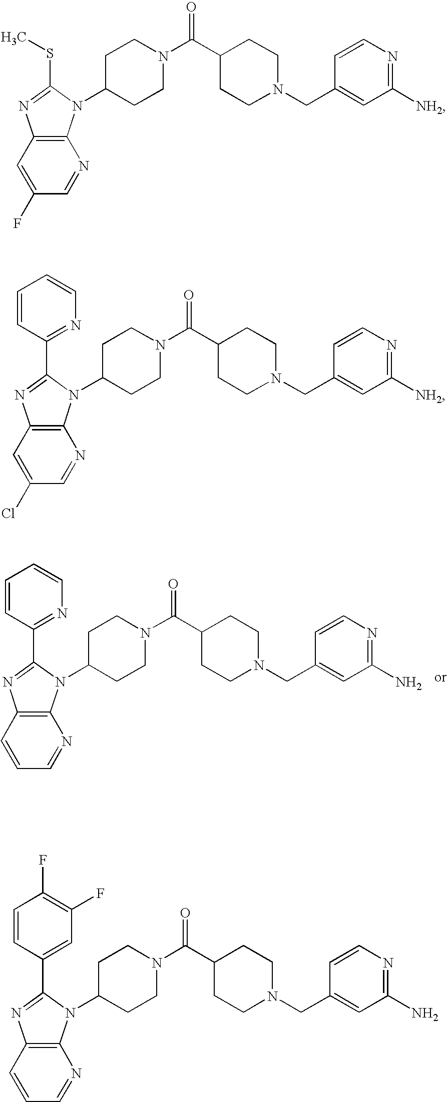
or a pharmaceutically acceptable salt, solvate, ester or prodrug thereof.

48. The method of claim 45, further comprising administering to the patient an additional analgesic agent that is not a compound of formula (I), wherein the amounts of the one or more compounds of Formula (I) and the additional analgesic agent are together effective to treat diabetes.

49. The method of claim 48, wherein the additional analgesic agent is acetaminophen, an NSAID, an opiate or a tricyclic antidepressant.

50. The method of claim 49, wherein the additional analgesic agent is acetaminophen or an NSAID.

51. The method of claim 50, wherein the NSAID is a salicylate, an arylalkanoic acid, a profen, a fenamic acid, a pyrazolidine derivative, a coxib, an oxicam or a sulfonanilide.

52. The method of claim 51, wherein the NSAID is aspirin, ibuprofen, naproxen, celecoxib, etoricoxib, lumiracoxib or parecoxib.

53. The method of claim 49, wherein the additional analgesic agent is an opiate.

54. The method of claim 53, wherein the opiate is an anilidopiperidine, a phenylpiperidine, a diphenylpropylamine derivative, a benzomorphane derivative, an oripavine derivative or a morphinane derivative.

55. The method of claim 54, wherein the opiate or is morphine, codeine, oxycodone, hydrocodone, diamorphine, pethidine, vicodin, percocet, percodan, norco, dilaudid, darvocet, lorcet, pentazocine, tramadol or fentanyl.

56. A composition comprising a compound of claim 1, an additional antidiabetic agent that is not a compound of formula (I), and a pharmaceutically acceptable carrier.

57. The composition of claim 55, wherein the additional antidiabetic agent is selected from a sulfonylurea, an insulin sensitizer, an α-glucosidase inhibitor, an insulin secretagogue, an anti-obesity agent, a meglitinide, insulin or an insulin-containing composition.

58. The composition of claim 56, wherein the additional antidiabetic agent is an insulin sensitizer or a sulfonylurea.

59. The composition of claim 57, wherein the insulin sensitizer is a PPAR activator.

60. The composition of claim 55, wherein the additional antidiabetic agent is an antiobesity agent.

61. The composition of claim 59, wherein the antiobesity agent is selected from: a neuropeptide Y antagonist, an MCR4 agonist, an MCH receptor antagonist, a protein hormone, an AMP kinase activator, and a lipase inhibitor.

62. The composition of claim 60, wherein antiobesity agent is orlistat, leptin, or adiponectin.

63. A composition comprising a compound of claim 1, an additional analgesic agent that is not a compound of formula (I), and a pharmaceutically acceptable carrier.

64. The composition of claim 63, wherein the additional analgesic agent is acetaminophen, an NSAID, an opiate or a tricyclic antidepressant.

65. The composition of claim 64, wherein the additional analgesic agent is acetaminophen or an NSAID.

66. The composition of claim 65, wherein the NSAID is a salicylate, an arylalkanoic acid, a profen, a fenamic acid, a pyrazolidine derivative, a coxib, an oxicam or a sulfonanilide.

67. The composition of claim 65, wherein the NSAID is aspirin, ibuprofen, naproxen, celecoxib, etoricoxib, lumiracoxib or parecoxib.

68. The composition of claim 63, wherein the additional analgesic agent is an opiate.

69. The composition of claim 68, wherein the opiate is an anilidopiperidine, a phenylpiperidine, a diphenylpropylamine derivative, a benzomorphane derivative, an oripavine derivative or a morphinane derivative.

70. The composition of claim 68, wherein the opiate is morphine, codeine, oxycodone, hydrocodone, diamorphine, pethidine, vicodin, percocet, percodan, norco, dilaudid, darvocet, lorcet, pentazocine, tramadol or fentanyl.
